(19) United States
(12) Patent Application Publication
(10) Pub. No.: US 2022/0184127 A1
(43) Pub. Date: Jun. 16, 2022

(54) HUMANIZED ANTI-DLL3 CHIMERIC ANTIGEN RECEPTORS AND USES THEREOF

(71) Applicant: Phanes Therapeutics, Inc., San Diego, CA (US)

(72) Inventors: Minghan Wang, San Diego, CA (US); Hui Zou, San Diego, CA (US); Haiqun Jia, San Diego, CA (US)

(21) Appl. No.: 17/593,087

(22) PCT Filed: Mar. 30, 2020
(86) PCT No.: PCT/US2020/025643

§ 371 Date: Sep. 9, 2021

Related U.S. Application Data
(60) Provisional application No. 62/830,598, filed on Apr. 8, 2019.
(60) Provisional application No. 62/861,377, filed on Jun. 14, 2019.
(60) Provisional application No. 62/896,790, filed on Sep. 6, 2019.
(60) Provisional application No. 62/928,615, filed on Oct. 31, 2019.

(51) Int. Cl. A61K 35/17; A61P 35/02; C07K 14/725; C07K 16/28

(52) U.S. Cl. CPC A61K 35/17; C07K 16/28; C07K 14/7051; A61P 35/02

(57) ABSTRACT

Chimeric antigen receptors (CARs) specific to DLL3, vectors encoding the DLL3 CAR, recombinant host cells comprising the DLL3 CAR (CAR-Ts or CAR-NKs), and methods of using the CAR-Ts or CAR-NKs to treat a disease associated with the expression of DLL3 thereof are described.

### CROSS REFERENCE TO RELATED APPLICATIONS

This application claims priority to U.S. Provisional Application No. 62/928,615, filed on Oct. 31, 2019; U.S. Provisional Application No. 62/896,790, filed Sep. 6, 2019; U.S. Provisional Application No. 62/861,377, filed on Jun. 14, 2019; and U.S. Provisional Application No. 62/830,598, filed on Apr. 8, 2019. Each disclosure is incorporated herein by reference in its entirety.

### FIELD OF THE INVENTION

This invention relates to anti-DLL3 chimeric antigen receptors (CARs), nucleic acids and expression vectors encoding the CARs, T cells engineered to express the CARs (CAR-T) and NK cells engineered to express the CARs (CAR-NK). Methods of making the CARs, methods of making the CAR-Ts/CAR-NKs, and methods of using the CAR-Ts/CAR-NKs to treat a disease associated with the expression of DLL3, including cancer, are also provided.

### REFERENCE TO SEQUENCE LISTING SUBMITTED ELECTRONICALLY

This application contains a sequence listing, which is submitted electronically via EFS-Web as an ASCII formatted sequence listing with a file name “065799.20WO1 Sequence Listing” and a creation date of Mar. 12, 2020 and having a size of 215 kb. The sequence listing submitted via EFS-Web is part of the specification and is herein incorporated by reference in its entirety.

### BACKGROUND OF THE INVENTION

The standard of care anti-cancer medicines provides significant benefits. Recently, the availability of immuno-oncology drugs such as anti-PD-1 mAbs, anti-PD-L1 mAbs and anti-CD3 bispecific T cell engagers has advanced the concept of leveraging and activating patients' immune system to fight various types of cancer. However, poor response, insufficient efficacy, and/or safety issues remain to be resolved. CAR-T (chimeric antigen receptor-T) cell therapies involve genetically engineering a patient's own immune cells, such as T cells, and redirecting them to a suitable cell surface antigen on cancer cells (Mayor et al., Immunotherapy 2016; 8:491-494). This approach has demonstrated success in patients suffering from chemorefractory B cell malignancies and other cancers (Pettitt et al., Mol Ther. 2018; 26:342-353). T cells can be engineered to possess specificity to one or more cancer cell surface targets/antigens to recognize and kill the cancer cell. The process includes transducing T cells with DNA or other genetic material encoding the chimeric antigen receptor (CAR), which comprises an extracellular antigen specific binding domain, such as one or more single chain variable fragments (scFv) of a monoclonal antibody, a hinge and transmembrane region, and an intracellular signaling domain (including one or more costimulatory domains and one or more activating domains) (Kochenderfer et al., Nat Rev Clin Oncol. 2013; 10:267-276). CAR-expressing immune cells, such as T cells and NK cells, can be used to treat various diseases, including liquid and solid tumors. Successful CAR-T cell therapies can specifically recognize and destroy targeted cells and maintain the ability to persist and proliferate over time.

Delta like canonical Notch ligand 3 (DLL3), also known as delta like 3 or delta like protein 3, is required for somite segmentation during early development (Dunwoodie et al., Development 2002; 129:1795-806). Unlike the mammalian Notch family ligands DLL1, DLL4, JAG1, and JAG2 which all activate Notch receptor signaling in trans (Ntziachristos et al., Cancer Cell 2014; 25(3):318-34), DLL3 is predominantly localized in the Golgi apparatus and is unable to activate Notch signaling (Chapman et al., Hum Mol Genet 2011; 20(5):905-16 and Geffers et al., J Cell Biol 2007; 178(3):465-76). During normal development, DLL3 inhibits both cis- and trans-acting Notch pathway activation by interacting with Notch and DLL1 (Chapman et al., Hum Mol Genet 2011; 20(5):905-16). DLL3 is normally either absent or present at very low levels in adult normal tissues except brain, but is overexpressed in lung cancer, testicular cancer, glioma and melanoma samples (Uhlen et al., Science 2017; 357(6352): eaan2507). Furthermore, DLL3 is detectable on the surface of small cell lung cancer (SCLC) and large cell neuroendocrine carcinoma (LCNEC) tumor cells (Saunders et al., Sci Transl Med 2015; 7(302):302ra136 and Sharma et al., Cancer Res. 2017; 77(14):3931-41), making it a potential target of monoclonal antibodies for cancer therapy. Therefore, DLL3 is an ideal target for CAR-T cell therapies to treat and cure DLL3-positive cancers.

### BRIEF SUMMARY OF THE INVENTION

In one general aspect, the invention relates to a chimeric antigen receptor (CAR) construct that induces T cell mediated cancer killing, wherein the CAR construct comprises at least one antigen binding domain that specifically binds DLL3, a hinge region, a transmembrane region, and an intracellular signaling domain.

Provided are isolated polynucleotides comprising a nucleic acid sequence encoding a chimeric antigen receptor (CAR). The CAR can comprise (a) an extracellular domain comprising at least one antigen binding domain that specifically binds DLL3; (b) a hinge region; (c) a transmembrane region; and (d) an intracellular signaling domain.

In certain embodiments, the antigen binding domain comprises a heavy chain complementarity determining region 1 (HCDR1), HCDR2, HCDR3, a light chain complementarity determining region 1 (LCDR1), LCDR2, and LCDR3, having the polypeptide sequences of:
(1) SEQ ID NOs: 25, 26, 27, 61, 62 and 63, respectively;
(2) SEQ ID NOs: 28, 29, 30, 64, 65 and 66, respectively;
(3) SEQ ID NOs: 31, 32, 33, 67, 68 and 69, respectively;
(4) SEQ ID NOs: 34, 35, 36, 70, 71 and 72, respectively;
(5) SEQ ID NOs: 37, 38, 39, 73, 74 and 75, respectively;
(6) SEQ ID NOs: 40, 41, 42, 76, 77 and 78, respectively;
(7) SEQ ID NOs: 43, 44, 45, 79, 80 and 81, respectively;
(8) SEQ ID NOs: 46, 47, 48, 82, 83 and 84, respectively;
(9) SEQ ID NOs: 49, 50, 51, 85, 86 and 87, respectively;
(10) SEQ ID NOs: 52, 53, 54, 88, 89 and 90, respectively;
(11) SEQ ID NOs: 55, 56, 57, 91, 92 and 93, respectively; or
(12) SEQ ID NOs: 58, 59, 60, 94, 95 and 96, respectively;
   wherein the antigen binding domain specifically binds DLL3, preferably human DLL3.

In certain embodiments, the antigen binding domain comprises a heavy chain complementarity determining region 1 (HCDR1), HCDR2, HCDR3, a light chain complementarity determining region 1 (LCDR1), LCDR2, and LCDR3, having the polypeptide sequences of:
(1) SEQ ID NOs: 97, 98, 99, 133, 134 and 135, respectively;
(2) SEQ ID NOs: 100, 101, 102, 136, 137 and 138, respectively;
(3) SEQ ID NOs: 103, 104, 105, 139, 140 and 141, respectively;
(4) SEQ ID NOs: 106, 107, 108, 142, 143 and 144, respectively;
(5) SEQ ID NOs: 109, 110, 111, 145, 146 and 147, respectively;
(6) SEQ ID NOs: 112, 113, 114, 148, 149 and 150, respectively;
(7) SEQ ID NOs: 115, 116, 117, 151, 152 and 153, respectively;
(8) SEQ ID NOs: 118, 119, 120, 154, 155 and 156, respectively;
(9) SEQ ID NOs: 121, 122, 123, 157, 158 and 159, respectively;
(10) SEQ ID NOs: 124, 125, 126, 160, 161 and 162, respectively;
(11) SEQ ID NOs: 127, 128, 129, 163, 164 and 165, respectively; or
(12) SEQ ID NOs: 130, 131, 132, 166, 167 and 168, respectively;
   wherein the antigen binding domain specifically binds DLL3, preferably human DLL3.

In certain embodiments, the antigen binding domain comprises a heavy chain variable region having a polypeptide sequence at least 95%, at least 96%, at least 97%, at least 98%, or at least 99% identical to SEQ ID NO: 1, 3, 5, 7, 9, 11, 13, 15, 17, 19, 21, or 23, or a light chain variable region having a polypeptide sequence at least 95%, at least 96%, at least 97%, at least 98%, or at least 99% identical to SEQ ID NO: 2, 4, 6, 8, 10, 12, 14, 16, 18, 20, 22, or 24.

In certain embodiments, the antigen binding domain comprises:
(1) a heavy chain variable region having the polypeptide sequence of SEQ ID NO:1, and a light chain variable region having the polypeptide sequence of SEQ ID NO:2;
(2) a heavy chain variable region having the polypeptide sequence of SEQ ID NO:3, and a light chain variable region having the polypeptide sequence of SEQ ID NO:4;
(3) a heavy chain variable region having the polypeptide sequence of SEQ ID NO:5, and a light chain variable region having the polypeptide sequence of SEQ ID NO:6;
(4) a heavy chain variable region having the polypeptide sequence of SEQ ID NO:7, and a light chain variable region having the polypeptide sequence of SEQ ID NO:8;
(5) a heavy chain variable region having the polypeptide sequence of SEQ ID NO:9, and a light chain variable region having the polypeptide sequence of SEQ ID NO:10;
(6) a heavy chain variable region having the polypeptide sequence of SEQ ID NO:11, and a light chain variable region having the polypeptide sequence of SEQ ID NO:12;
(7) a heavy chain variable region having the polypeptide sequence of SEQ ID NO:13, and a light chain variable region having the polypeptide sequence of SEQ ID NO:14;
(8) a heavy chain variable region having the polypeptide sequence of SEQ ID NO:15, and a light chain variable region having the polypeptide sequence of SEQ ID NO:16;
(9) a heavy chain variable region having the polypeptide sequence of SEQ ID NO:17, and a light chain variable region having the polypeptide sequence of SEQ ID NO:18;
(10) a heavy chain variable region having the polypeptide sequence of SEQ ID NO:19, and a light chain variable region having the polypeptide sequence of SEQ ID NO:20;
(11) a heavy chain variable region having the polypeptide sequence of SEQ ID NO:21, and a light chain variable region having the polypeptide sequence of SEQ ID NO:22; or
(12) a heavy chain variable region having the polypeptide sequence of SEQ ID NO:23, and a light chain variable region having the polypeptide sequence of SEQ ID NO:24.

In certain embodiments, the antigen binding domain is humanized and comprises a heavy chain variable region having a polypeptide sequence at least 95%, at least 96%, at least 97%, at least 98%, or at least 99% identical to any one of SEQ ID NOs: 170, 175-209 or 248-255, or a light chain variable region having a polypeptide sequence at least 95%, at least 96%, at least 97%, at least 98%, or at least 99% identical to any one of SEQ ID NOs: 171-174, 210-240 or 256-264.

In certain embodiments, the antigen binding domain is humanized and comprises:
(1) a heavy chain variable region having the polypeptide sequence of SEQ ID NO:170, and a light chain variable region having the polypeptide sequence of SEQ ID NO:171;
(2) a heavy chain variable region having the polypeptide sequence of SEQ ID NO:170, and a light chain variable region having the polypeptide sequence of SEQ ID NO:172;
(3) a heavy chain variable region having the polypeptide sequence of SEQ ID NO:170, and a light chain variable region having the polypeptide sequence of SEQ ID NO:173;
(4) a heavy chain variable region having the polypeptide sequence of SEQ ID NO:183, and a light chain variable region having the polypeptide sequence of SEQ ID NO:217;
(5) a heavy chain variable region having the polypeptide sequence of SEQ ID NO:183, and a light chain variable region having the polypeptide sequence of SEQ ID NO:218;
(6) a heavy chain variable region having the polypeptide sequence of SEQ ID NO:184, and a light chain variable region having the polypeptide sequence of SEQ ID NO:217;
(7) a heavy chain variable region having the polypeptide sequence of SEQ ID NO:184, and a light chain variable region having the polypeptide sequence of SEQ ID NO:218;
(8) a heavy chain variable region having the polypeptide sequence of SEQ ID NO:198, and a light chain variable region having the polypeptide sequence of SEQ ID NO:229;
(9) a heavy chain variable region having the polypeptide sequence of SEQ ID NO:200, and a light chain variable region having the polypeptide sequence of SEQ ID NO:229;
(10) a heavy chain variable region having the polypeptide sequence of SEQ ID NO:198, and a light chain variable region having the polypeptide sequence of SEQ ID NO:231;
(11) a heavy chain variable region having the polypeptide sequence of SEQ ID NO:200, and a light chain variable region having the polypeptide sequence of SEQ ID NO:231;
(12) a heavy chain variable region having the polypeptide sequence of SEQ ID NO:201, and a light chain variable region having the polypeptide sequence of SEQ ID NO:229;
(13) a heavy chain variable region having the polypeptide sequence of SEQ ID NO:201, and a light chain variable region having the polypeptide sequence of SEQ ID NO:230;
(14) a heavy chain variable region having the polypeptide sequence of SEQ ID NO:201, and a light chain variable region having the polypeptide sequence of SEQ ID NO:231;
(15) a heavy chain variable region having the polypeptide sequence of SEQ ID NO:175, and a light chain variable region having the polypeptide sequence of SEQ ID NO:210;
(16) a heavy chain variable region having the polypeptide sequence of SEQ ID NO:175, and a light chain variable region having the polypeptide sequence of SEQ ID NO:211;
(17) a heavy chain variable region having the polypeptide sequence of SEQ ID NO:175, and a light chain variable region having the polypeptide sequence of SEQ ID NO:212;
(18) a heavy chain variable region having the polypeptide sequence of SEQ ID NO:176, and a light chain variable region having the polypeptide sequence of SEQ ID NO:210;
(19) a heavy chain variable region having the polypeptide sequence of SEQ ID NO:176, and a light chain variable region having the polypeptide sequence of SEQ ID NO:211;
(20) a heavy chain variable region having the polypeptide sequence of SEQ ID NO:176, and a light chain variable region having the polypeptide sequence of SEQ ID NO:212;
(21) a heavy chain variable region having the polypeptide sequence of SEQ ID NO:177, and a light chain variable region having the polypeptide sequence of SEQ ID NO:210;
(22) a heavy chain variable region having the polypeptide sequence of SEQ ID NO:177, and a light chain variable region having the polypeptide sequence of SEQ ID NO:211;
(23) a heavy chain variable region having the polypeptide sequence of SEQ ID NO:178, and a light chain variable region having the polypeptide sequence of SEQ ID NO:210;
(24) a heavy chain variable region having the polypeptide sequence of SEQ ID NO:178, and a light chain variable region having the polypeptide sequence of SEQ ID NO:211;
(25) a heavy chain variable region having the polypeptide sequence of SEQ ID NO:177, and a light chain variable region having the polypeptide sequence of SEQ ID NO:211;
(26) a heavy chain variable region having the polypeptide sequence of SEQ ID NO:177, and a light chain variable region having the polypeptide sequence of SEQ ID NO:212;
(27) a heavy chain variable region having the polypeptide sequence of SEQ ID NO:178, and a light chain variable region having the polypeptide sequence of SEQ ID NO:212;
(28) a heavy chain variable region having the polypeptide sequence of SEQ ID NO:179, and a light chain variable region having the polypeptide sequence of SEQ ID NO:213;
(29) a heavy chain variable region having the polypeptide sequence of SEQ ID NO:179, and a light chain variable region having the polypeptide sequence of SEQ ID NO:214;
(30) a heavy chain variable region having the polypeptide sequence of SEQ ID NO:179, and a light chain variable region having the polypeptide sequence of SEQ ID NO:215;
(31) a heavy chain variable region having the polypeptide sequence of SEQ ID NO:180, and a light chain variable region having the polypeptide sequence of SEQ ID NO:213;
(32) a heavy chain variable region having the polypeptide sequence of SEQ ID NO:180, and a light chain variable region having the polypeptide sequence of SEQ ID NO:214;
(33) a heavy chain variable region having the polypeptide sequence of SEQ ID NO:180, and a light chain variable region having the polypeptide sequence of SEQ ID NO:215;
(34) a heavy chain variable region having the polypeptide sequence of SEQ ID NO:181, and a light chain variable region having the polypeptide sequence of SEQ ID NO:213;
(35) a heavy chain variable region having the polypeptide sequence of SEQ ID NO:181, and a light chain variable region having the polypeptide sequence of SEQ ID NO:214;
(36) a heavy chain variable region having the polypeptide sequence of SEQ ID NO:182, and a light chain variable region having the polypeptide sequence of SEQ ID NO:215;
(37) a heavy chain variable region having the polypeptide sequence of SEQ ID NO:202, and a light chain variable region having the polypeptide sequence of SEQ ID NO:232;
(38) a heavy chain variable region having the polypeptide sequence of SEQ ID NO:202, and a light chain variable region having the polypeptide sequence of SEQ ID NO:233;
(39) a heavy chain variable region having the polypeptide sequence of SEQ ID NO:202, and a light chain variable region having the polypeptide sequence of SEQ ID NO:234;
(40) a heavy chain variable region having the polypeptide sequence of SEQ ID NO:203, and a light chain variable region having the polypeptide sequence of SEQ ID NO:232;
(41) a heavy chain variable region having the polypeptide sequence of SEQ ID NO:203, and a light chain variable region having the polypeptide sequence of SEQ ID NO:233;
(42) a heavy chain variable region having the polypeptide sequence of SEQ ID NO:203, and a light chain variable region having the polypeptide sequence of SEQ ID NO:234;
(43) a heavy chain variable region having the polypeptide sequence of SEQ ID NO:204, and a light chain variable region having the polypeptide sequence of SEQ ID NO:234;
(44) a heavy chain variable region having the polypeptide sequence of SEQ ID NO:208, and a light chain variable region having the polypeptide sequence of SEQ ID NO:239;
(45) a heavy chain variable region having the polypeptide sequence of SEQ ID NO:208, and a light chain variable region having the polypeptide sequence of SEQ ID NO:240;
(46) a heavy chain variable region having the polypeptide sequence of SEQ ID NO:253, and a light chain variable region having the polypeptide sequence of SEQ ID NO:261; or
(47) a heavy chain variable region having the polypeptide sequence of SEQ ID NO:255, and a light chain variable region having the polypeptide sequence of SEQ ID NO:263.

In certain embodiments, the antigen binding domain is a single chain variable fragment (scFv) that specifically binds DLL3, preferably human DLL3.

In certain embodiments, the antigen binding domain is a humanized single chain variable fragment (scFv) that specifically binds DLL3, preferably human DLL3.

In certain embodiments, the single chain variable fragment (scFv) comprises a polypeptide sequence at least 95% identical to any one of SEQ ID NOs: 241-247 or 265-286.

In certain embodiments, the chimeric antigen receptor (CAR) comprises one or more antigen binding domains.

In certain embodiments, the intracellular signaling domain comprises one or more costimulatory domains and one or more activating domains.

Also provided are chimeric antigen receptors (CARs) encoded by the isolated polynucleotides of the invention.

Also provided are vectors comprising the isolated polynucleotides comprising nucleic acids encoding the CARs of the invention.

Also provided are host cells comprising the vectors of the invention.

In certain embodiments, the host cell is a T cell, preferably a human T cell. In certain embodiments, the host cell is a NK cell, preferably a human NK cell. The T cell or NK cell can, for example, be engineered to express the CAR of the invention to treat diseases such as cancer.

Also provided are methods of making a host cell expressing a chimeric antigen receptor (CAR) of the invention. The methods comprise transducing a T cell or a NK cell with a vector comprising the isolated nucleic acids encoding the CARs of the invention.

Also provided are methods of producing a CAR-T cell or CAR-NK cell of the invention. The methods comprise culturing T cells or NK cells comprising the isolated polynucleotide comprising a nucleic acid encoding a chimeric antigen receptor (CAR) of the invention under conditions to produce the CAR-T cell or CAR-NK cell, and recovering the CAR-T cell or CAR-NK cell.

Also provided are methods of generating a population of RNA-engineered cells comprising a chimeric antigen receptor (CAR) of the invention. The methods comprise contacting a cell with the isolated polynucleotide comprising a nucleic acid encoding a chimeric antigen receptor (CAR) of the invention, wherein the isolated polynucleotide is an in vitro transcribed RNA or synthetic RNA.

Also provided are methods of treating cancer in a subject in need thereof, comprising administering to the subject the CAR-T cells and/or CAR-NK cells of the invention. The cancer can be any liquid or solid cancer, for example, it can be selected from, but not limited to, a lung cancer such as small cell lung cancer (SCLC), large cell neuroendocrine carcinoma (LCNEC), a gastric cancer, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, and other solid tumors, and a non-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors. Also provided are methods of treating cancer in a subject in need thereof, comprising administering to the subject a pharmaceutical composition of the invention.

In certain embodiments, the methods of treating cancer in a subject in need thereof further comprise administering to the subject in need thereof an agent that increases the efficacy of a cell expressing a CAR molecule.

In certain embodiments, the methods of treating cancer in a subject in need thereof further comprise administering to the subject in need thereof an agent that ameliorates one or more side effects associated with administration of a cell expressing a CAR molecule.

In certain embodiments, the methods of treating cancer in a subject in need thereof further comprise administering to the subject in need thereof an agent that treats the disease associated with DLL3.

Also provided are humanized anti-DLL3 monoclonal antibodies or antigen-binding fragments, wherein the antibodies or antigen-binding fragments thereof comprise a heavy chain variable region having a polypeptide sequence at least 95% identical to any one of SEQ ID NOs: 170, 175-209 or 248-255, or a light chain variable region having a polypeptide sequence at least 95% identical to any one of SEQ ID NOs: 171-174, 210-240 or 256-264.

In certain embodiments, the humanized anti-DLL3 monoclonal antibodies or antigen-binding fragments thereof comprise:
(1) a heavy chain variable region having the polypeptide sequence of SEQ ID NO:170, and a light chain variable region having the polypeptide sequence of SEQ ID NO:171;
(2) a heavy chain variable region having the polypeptide sequence of SEQ ID NO:170, and a light chain variable region having the polypeptide sequence of SEQ ID NO:172;
(3) a heavy chain variable region having the polypeptide sequence of SEQ ID NO:170, and a light chain variable region having the polypeptide sequence of SEQ ID NO:173.
(4) a heavy chain variable region having the polypeptide sequence of SEQ ID NO:183, and a light chain variable region having the polypeptide sequence of SEQ ID NO:217;
(5) a heavy chain variable region having the polypeptide sequence of SEQ ID NO:183, and a light chain variable region having the polypeptide sequence of SEQ ID NO:218;
(6) a heavy chain variable region having the polypeptide sequence of SEQ ID NO:184, and a light chain variable region having the polypeptide sequence of SEQ ID NO:217;
(7) a heavy chain variable region having the polypeptide sequence of SEQ ID NO:184, and a light chain variable region having the polypeptide sequence of SEQ ID NO:218;
(8) a heavy chain variable region having the polypeptide sequence of SEQ ID NO:198, and a light chain variable region having the polypeptide sequence of SEQ ID NO:229;
(9) a heavy chain variable region having the polypeptide sequence of SEQ ID NO:200, and a light chain variable region having the polypeptide sequence of SEQ ID NO:229;
(10) a heavy chain variable region having the polypeptide sequence of SEQ ID NO:198, and a light chain variable region having the polypeptide sequence of SEQ ID NO:231;
(11) a heavy chain variable region having the polypeptide sequence of SEQ ID NO:200, and a light chain variable region having the polypeptide sequence of SEQ ID NO:231;
(12) a heavy chain variable region having the polypeptide sequence of SEQ ID NO:201, and a light chain variable region having the polypeptide sequence of SEQ ID NO:229;
(13) a heavy chain variable region having the polypeptide sequence of SEQ ID NO:201, and a light chain variable region having the polypeptide sequence of SEQ ID NO:230;
(14) a heavy chain variable region having the polypeptide sequence of SEQ ID NO:201, and a light chain variable region having the polypeptide sequence of SEQ ID NO:231;
(15) a heavy chain variable region having the polypeptide sequence of SEQ ID NO:175, and a light chain variable region having the polypeptide sequence of SEQ ID NO:210;
(16) a heavy chain variable region having the polypeptide sequence of SEQ ID NO:175, and a light chain variable region having the polypeptide sequence of SEQ ID NO:211;
(17) a heavy chain variable region having the polypeptide sequence of SEQ ID NO:175, and a light chain variable region having the polypeptide sequence of SEQ ID NO:212;
(18) a heavy chain variable region having the polypeptide sequence of SEQ ID NO:176, and a light chain variable region having the polypeptide sequence of SEQ ID NO:210;
(19) a heavy chain variable region having the polypeptide sequence of SEQ ID NO:176, and a light chain variable region having the polypeptide sequence of SEQ ID NO:211;
(20) a heavy chain variable region having the polypeptide sequence of SEQ ID NO:176, and a light chain variable region having the polypeptide sequence of SEQ ID NO:212;
(21) a heavy chain variable region having the polypeptide sequence of SEQ ID NO:177, and a light chain variable region having the polypeptide sequence of SEQ ID NO:210;
(22) a heavy chain variable region having the polypeptide sequence of SEQ ID NO:177, and a light chain variable region having the polypeptide sequence of SEQ ID NO:211;
(23) a heavy chain variable region having the polypeptide sequence of SEQ ID NO:178, and a light chain variable region having the polypeptide sequence of SEQ ID NO:210;
(24) a heavy chain variable region having the polypeptide sequence of SEQ ID NO:178, and a light chain variable region having the polypeptide sequence of SEQ ID NO:211;
(25) a heavy chain variable region having the polypeptide sequence of SEQ ID NO:177, and a light chain variable region having the polypeptide sequence of SEQ ID NO:211;
(26) a heavy chain variable region having the polypeptide sequence of SEQ ID NO:177, and a light chain variable region having the polypeptide sequence of SEQ ID NO:212;
(27) a heavy chain variable region having the polypeptide sequence of SEQ ID NO:178, and a light chain variable region having the polypeptide sequence of SEQ ID NO:212;
(28) a heavy chain variable region having the polypeptide sequence of SEQ ID NO:179, and a light chain variable region having the polypeptide sequence of SEQ ID NO:213;
(29) a heavy chain variable region having the polypeptide sequence of SEQ ID NO:179, and a light chain variable region having the polypeptide sequence of SEQ ID NO:214;
(30) a heavy chain variable region having the polypeptide sequence of SEQ ID NO:179, and a light chain variable region having the polypeptide sequence of SEQ ID NO:215;
(31) a heavy chain variable region having the polypeptide sequence of SEQ ID NO:180, and a light chain variable region having the polypeptide sequence of SEQ ID NO:213;
(32) a heavy chain variable region having the polypeptide sequence of SEQ ID NO:180, and a light chain variable region having the polypeptide sequence of SEQ ID NO:214;
(33) a heavy chain variable region having the polypeptide sequence of SEQ ID NO:180, and a light chain variable region having the polypeptide sequence of SEQ ID NO:215;
(34) a heavy chain variable region having the polypeptide sequence of SEQ ID NO:181, and a light chain variable region having the polypeptide sequence of SEQ ID NO:213;
(35) a heavy chain variable region having the polypeptide sequence of SEQ ID NO:181, and a light chain variable region having the polypeptide sequence of SEQ ID NO:214;
(36) a heavy chain variable region having the polypeptide sequence of SEQ ID NO:182, and a light chain variable region having the polypeptide sequence of SEQ ID NO:215;
(37) a heavy chain variable region having the polypeptide sequence of SEQ ID NO:202, and a light chain variable region having the polypeptide sequence of SEQ ID NO:232;
(38) a heavy chain variable region having the polypeptide sequence of SEQ ID NO:202, and a light chain variable region having the polypeptide sequence of SEQ ID NO:233;
(39) a heavy chain variable region having the polypeptide sequence of SEQ ID NO:202, and a light chain variable region having the polypeptide sequence of SEQ ID NO:234;
(40) a heavy chain variable region having the polypeptide sequence of SEQ ID NO:203, and a light chain variable region having the polypeptide sequence of SEQ ID NO:232;
(41) a heavy chain variable region having the polypeptide sequence of SEQ ID NO:203, and a light chain variable region having the polypeptide sequence of SEQ ID NO:233;
(42) a heavy chain variable region having the polypeptide sequence of SEQ ID NO:203, and a light chain variable region having the polypeptide sequence of SEQ ID NO:234;
(43) a heavy chain variable region having the polypeptide sequence of SEQ ID NO:204, and a light chain variable region having the polypeptide sequence of SEQ ID NO:234;
(44) a heavy chain variable region having the polypeptide sequence of SEQ ID NO:208, and a light chain variable region having the polypeptide sequence of SEQ ID NO:239;
(45) a heavy chain variable region having the polypeptide sequence of SEQ ID NO:208, and a light chain variable region having the polypeptide sequence of SEQ ID NO:240;
(46) a heavy chain variable region having the polypeptide sequence of SEQ ID NO:253, and a light chain variable region having the polypeptide sequence of SEQ ID NO:261; or
(47) a heavy chain variable region having the polypeptide sequence of SEQ ID NO:255, and a light chain variable region having the polypeptide sequence of SEQ ID NO:263.

In certain embodiments, the humanized anti-DLL3 monoclonal antibody or antigen-binding fragment is capable of inducing effector-mediated tumor cell lysis, mediating the recruitment of conjugated drugs, and/or forms a bispecific antibody with another monoclonal antibody or antigen-binding fragment with a cancer-killing effect.

Also provided are isolated nucleic acids encoding the humanized anti-DLL3 monoclonal antibodies or antigen-binding fragments thereof of the invention.

Also provided are vectors comprising the isolated nucleic acids encoding the humanized anti-DLL3 monoclonal antibodies or antigen-binding fragments thereof of the invention.

Also provided are host cells comprising the vectors comprising the isolated nucleic acids encoding the humanized anti-DLL3 monoclonal antibodies or antigen-binding fragments thereof of the invention.

Also provided is a pharmaceutical composition comprising the humanized anti-DLL3 monoclonal antibody or antigen-binding fragment thereof of the invention and a pharmaceutically acceptable carrier.

Also provided are methods of targeting DLL3 on a cancer cell surface in a subject in need thereof, comprising administering to the subject in need thereof a pharmaceutical composition comprising the humanized anti-DLL3 monoclonal antibody or antigen-binding fragment thereof of the invention.

Also provided are methods of treating cancer in a subject in need thereof, comprising administering to the subject the pharmaceutical composition comprising the humanized anti-DLL3 monoclonal antibody or antigen-binding fragment thereof of the invention. The cancer can be any liquid or solid cancer, for example, it can be selected from, but not limited to, a lung cancer such as small cell lung cancer (SCLC), large cell neuroendocrine carcinoma (LCNEC), a gastric cancer, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, and other solid tumors, and a non-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

Also provided are methods of producing the humanized anti-DLL3 monoclonal antibody or antigen-binding fragment thereof of the invention, comprising culturing a cell comprising a nucleic acid encoding the monoclonal antibody or antigen-binding fragment under conditions to produce the monoclonal antibody or antigen-binding fragment, and recovering the antibody or antigen-binding fragment from the cell or culture.

Also provided are methods of producing a pharmaceutical composition comprising the humanized anti-DLL3 monoclonal antibody or antigen-binding fragment thereof of the invention, comprising combining the monoclonal antibody or antigen-binding fragment with a pharmaceutically acceptable carrier to obtain the pharmaceutical composition.

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

FIGS. 1A-1Q show the binding of humanized mAbs to immobilized recombinant human DLL3 in an ELISA assay.

FIGS. 2A-2I show the binding of single chain variable fragments (scFvs) to immobilized recombinant human DLL3 protein by ELISA.

FIGS. 3A-3F show the binding of scFvs to HEK293-huDLL3 cells stably expressing human DLL3. The experiment was carried out by FACS analysis.

FIG. 4 shows the tumor cell killing activity of the CART cells assembled with an anti-DLL3 scFv. Mock transfected T cells were used as control.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of” or variations such as “consist essentially of” or “consisting essentially of” as used throughout the specification and claims, indicate the inclusion of any recited integer or group of integers, and the optional inclusion of any recited integer or group of integers that do not materially change the basic or novel properties of the specified method, structure or composition. See M. P. E. P. § 2111.03.

As used herein, “subject” means any animal, preferably a mammal, most preferably a human. The term “mammal” as used herein, encompasses any mammal. Examples of mammals include, but are not limited to, cows, horses, sheep, pigs, cats, dogs, mice, rats, rabbits, guinea pigs, monkeys, humans, etc., more preferably a human.

The words “right,” “left,” “lower,” and “upper” designate directions in the drawings to which reference is made.

It should also be understood that the terms “about,” “approximately,” “generally,” “substantially,” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The terms “identical” or percent “identity,” in the context of two or more nucleic acids or polypeptide sequences (e.g., chimeric antigen receptors (CARs) comprising antigen binding domains specific for DLL3 and polynucleotides that encode them, DLL3 polypeptides and DLL3 polynucleotides that encode them), refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 1981; 2:482, by the homology alignment algorithm of Needleman & Wunsch, J. Mol. Biol. 1970; 48:443, by the search for similarity method of Pearson & Lipman, Proc. Nat'l. Acad. Sci. USA 1988; 85:2444, by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Current Protocols in Molecular Biology, F. M. Ausubel et al., eds., Current Protocols, a joint venture between Greene Publishing Associates, Inc. and John Wiley & Sons, Inc., 1995 Supplement (Ausubel)).

Examples of algorithms that are suitable for determining percent sequence identity and sequence similarity are the BLAST and BLAST 2.0 algorithms, which are described in Altschul et al., J. Mol. Biol. 1990; 215: 403-410 and Altschul et al., Nucleic Acids Res. 1997; 25: 3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 1989; 89:10915).

In addition to calculating percent sequence identity, the BLAST algorithm also performs a statistical analysis of the similarity between two sequences (see, e.g., Karlin & Altschul, Proc. Nat'l. Acad. Sci. USA 1993; 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

As used herein, the term “isolated” means a biological component (such as a nucleic acid, peptide or protein) has been substantially separated, produced apart from, or purified away from other biological components of the organism in which the component naturally occurs, i.e., other chromosomal and extrachromosomal DNA and RNA, and proteins. Nucleic acids, peptides and proteins that have been “isolated” thus include nucleic acids and proteins purified by standard purification methods. “Isolated” nucleic acids, peptides and proteins can be part of a composition and still be isolated if the composition is not part of the native environment of the nucleic acid, peptide, or protein. The term also embraces nucleic acids, peptides and proteins prepared by recombinant expression in a host cell as well as chemically synthesized nucleic acids.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

As used herein, the term “vector” is a replicon in which another nucleic acid segment can be operably inserted so as to bring about the replication or expression of the segment.

As used herein, the term “host cell” refers to a cell comprising a nucleic acid molecule of the invention. The “host cell” can be any type of cell, e.g., a primary cell, a cell in culture, or a cell from a cell line. In one embodiment, a “host cell” is a cell transfected or transduced with a nucleic acid molecule of the invention. In another embodiment, a “host cell” is a progeny or potential progeny of such a transfected or transduced cell. A progeny of a cell may or may not be identical to the parent cell, e.g., due to mutations or environmental influences that can occur in succeeding generations or integration of the nucleic acid molecule into the host cell genome.

The term “expression” as used herein, refers to the biosynthesis of a gene product. The term encompasses the transcription of a gene into RNA. The term also encompasses translation of RNA into one or more polypeptides, and further encompasses all naturally occurring post-transcriptional and post-translational modifications. The expressed CAR can be within the cytoplasm of a host cell, into the extracellular milieu such as the growth medium of a cell culture or anchored to the cell membrane.

As used herein, the term “immune cell” or “immune effector cell” refers to a cell that is involved in an immune response, e.g., in the promotion of an immune effector response. Examples of immune cells include T cells, B cells, natural killer (NK) cells, mast cells, and myeloid-derived phagocytes. According to particular embodiments, the engineered immune cells are T cells, and are referred to as CAR-T cells because they are engineered to express CARs of the invention.

As used herein, the term “engineered immune cell” refers to an immune cell, also referred to as an immune effector cell, that has been genetically modified by the addition of extra genetic material in the form of DNA or RNA to the total genetic material of the cell. According to embodiments herein, the engineered immune cells have been genetically modified to express a CAR construct according to the invention.

### Chimeric Antigen Receptor (CAR)

As used herein, the term “chimeric antigen receptor” (CAR) refers to a recombinant polypeptide comprising at least an extracellular domain that binds specifically to an antigen or a target, a transmembrane domain and an intracellular T cell receptor-activating signaling domain. Engagement of the extracellular domain of the CAR with the target antigen on the surface of a target cell results in clustering of the CAR and delivers an activation stimulus to the CAR-containing cell. CARs redirect the specificity of immune effector cells and trigger proliferation, cytokine production, phagocytosis and/or production of molecules that can mediate cell death of the target antigen-expressing cell in a major histocompatibility (MHC)-independent manner.

In one aspect, the CAR comprises an antigen binding domain, a hinge region, a costimulatory domain, an activating domain and a transmembrane region. In one aspect, the CAR comprises an antigen binding domain, a hinge region, two costimulatory domains, an activating domain and a transmembrane region. In one aspect, the CAR comprises two antigen binding domains, a hinge region, a costimulatory domain, an activating domain and a transmembrane region. In one aspect, the CAR comprises two antigen binding domains, a hinge region, two costimulatory domains, an activating domain and a transmembrane region.

As used herein, the term “signal peptide” refers to a leader sequence at the amino-terminus (N-terminus) of a nascent CAR protein, which co-translationally or post-translationally directs the nascent protein to the endoplasmic reticulum and subsequent surface expression.

As used herein, the term “extracellular antigen binding domain,” “extracellular domain,” or “extracellular ligand binding domain” refers to the part of a CAR that is located outside of the cell membrane and is capable of binding to an antigen, target or ligand.

As used herein, the term “hinge region” refers to the part of a CAR that connects two adjacent domains of the CAR protein, e.g., the extracellular domain and the transmembrane domain.

As used herein, the term “transmembrane domain” refers to the portion of a CAR that extends across the cell membrane and anchors the CAR to cell membrane. The “transmembrane domain” can also be referred to as a “transmembrane region.”

### Costimulatory Domains

As used herein, chimeric antigen receptors can incorporate costimulatory (signaling) domains to increase their potency. A costimulatory (signaling) domain can be derived from a costimulatory molecule. Costimulatory molecules are cell surface molecules other than antigen receptors or their ligands that are required for an efficient immune response. Costimulatory domains can be derived from costimulatory molecules, which can include, but are not limited to, CD28, CD28T, OX40, 4-1BB/CD137, CD2, CD3 (alpha, beta, delta, epsilon, gamma, zeta), CD4, CD5, CD7, CD9, CD16, CD22, CD27, CD30, CD33, CD37, CD40, CD45, CD64, CD80, CD86, CD134, CD137, CD154, programmed death-1 (PD-1), inducible T cell costimulator (ICOS), lymphocyte function-associated antigen-1 (LFA-1; CD11a and CD18), CD247, CD276 (B7-H3), LIGHT (tumor necrosis factor superfamily member 14; TNFSF14), NKG2C, Ig alpha (CD79a), DAP10, Fc gamma receptor, MHC class I molecule, TNFR, integrin, signaling lymphocytic activation molecule, BTLA, Toll ligand receptor, ICAM-1, CDS, GITR, BAFFR, LIGHT, HVEM (LIGHTR), KIRDS2, SLAMF7, NKp80 (KLRF1), NKp44, NKp30, NKp46, CD19, CD8 alpha, CD8 beta, IL-2R beta, IL-2R gamma, IL-7R alpha, ITGA4, VLA1, CD49a, IA4, CD49D, ITGA6, VLA-6, CD49f, ITGAD, ITGAE, CD103, ITGAL, CD1a, CD1b, CD1c, CD1d, ITGAM, ITGAX, ITGB1, CD29, ITGB2 (CD18), ITGB7, NKG2D, TNFR2, TRANCE/RANKL, DNAM1 (CD226), SLAMF4 (CD244, 2B4), CD84, CD96 (Tactile), CEACAM1, CRTAM, Ly9 (CD229), CD 160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, CD83 ligand, cytokine receptor, activating NK cell receptors, or fragments or any combination thereof.

### Activating Domains

As used herein, chimeric antigen receptors can comprise activating domains. Activating domains can include, but are not limited to, CD3. CD3 is an element of the T cell receptor on native T cells and has been shown to be an important intracellular activating element in CARs. In a preferred embodiment, the CD3 is CD3 zeta.

### Hinge Region

As described herein, the chimeric antigen receptor can comprise a hinge region. This is a portion of the extracellular domain, sometimes referred to as a “spacer” region. A variety of hinges can be employed in accordance with the invention, including costimulatory molecules, as discussed above, immunoglobulin (Ig) sequences, or other suitable molecules to achieve the desired special distance from the target cell. In some embodiments, the entire extracellular region comprises a hinge region.

### Transmembrane Region

As used herein, chimeric antigen receptors (CARs) can comprise a transmembrane region/domain. The CAR can be designed to comprise a transmembrane domain that is fused to the extracellular domain of the CAR. It can similarly be fused to the intracellular domain of the CAR. In one embodiment, the transmembrane domain that is naturally associated with one of the domains in a CAR is used. In some instances, the transmembrane domain can be selected or modified by amino acid substitution to avoid binding of such domains to the transmembrane domains of the same or different surface membrane proteins to minimize interactions with other members of the receptor complex. The transmembrane domain may be derived either from a natural or from a synthetic source. Where the source is natural, the domain may be derived from any membrane-bound or transmembrane protein. Transmembrane regions of particular use in this invention can be derived from (i.e. comprise or engineered from), but are not limited to, CD28, CD28T, OX40, 4-1BB/CD137, CD2, CD3 (alpha, beta, delta, epsilon, gamma, zeta), CD4, CD5, CD7, CD9, CD16, CD22, CD27, CD30, CD33, CD37, CD40, CD45, CD64, CD80, CD86, CD134, CD137, CD154, programmed death-1 (PD-1), inducible T cell costimulator (ICOS), lymphocyte function-associated antigen-1 (LFA-1; CD11a and CD18), CD247, CD276 (B7-H3), LIGHT (tumor necrosis factor superfamily member 14; TNFSF14), NKG2C, Ig alpha (CD79a), DAP10, Fc gamma receptor, MHC class I molecule, TNFR, integrin, signaling lymphocytic activation molecule, BTLA, Toll ligand receptor, ICAM-1, CDS, GITR, BAFFR, LIGHT, HVEM (LIGHTR), KIRDS2, SLAMF7, NKp80 (KLRF1), NKp44, NKp30, NKp46, CD19, CD8 alpha, CD8 beta, IL-2R beta, IL-2R gamma, IL-7R alpha, ITGA4, VLA1, CD49a, IA4, CD49D, ITGA6, VLA-6, CD49f, ITGAD, ITGAE, CD103, ITGAL, CD1a, CD1b, CD1c, CD1d, ITGAM, ITGAX, ITGB1, CD29, ITGB2 (CD18), ITGB7, NKG2D, TNFR2, TRANCE/RANKL, DNAM1 (CD226), SLAMF4 (CD244, 2B4), CD84, CD96 (Tactile), CEACAM1, CRTAM, Ly9 (CD229), CD 160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, CD83 ligand, cytokine receptor, activating NK cell receptors, an immunoglobulin protein, or fragments or any combination thereof.

### Immune Cells

According to particular aspects, the invention provides cells that are immune cells that comprise the isolated polynucleotides or vectors comprising the isolated polynucleotides comprising the nucleotide sequence encoding the CAR are provided herein. The immune cells comprising the isolated polynucleotides and/or vectors of the invention can be referred to as “engineered immune cells.” Preferably, the engineered immune cells are derived from a human (are of human origin prior to being made recombinant).

The engineered immune cells can, for example, be cells of the lymphoid lineage. Non-limiting examples of cells of the lymphoid lineage can include T cells and Natural Killer (NK) cells. T cells express the T cell receptor (TCR), with most cells expressing α and β chains and a smaller population expressing γ and δ chains. T cells useful as engineered immune cells of the invention can be CD4⁺ or CD8⁺ and can include, but are not limited to, T helper cells (CD4⁺), cytotoxic T cells (also referred to as cytotoxic T lymphocytes, CTL; CD8⁺ cells), and memory T cells, including central memory T cells, stem-like memory T cells, and effector memory T cells, natural killer T cells, mucosal associated invariant T cells, and γδ T cells. Other exemplary immune cells include, but are not limited to, macrophages, antigen presenting cells (APCs), or any immune cell that expresses an inhibitor of a cell-mediated immune response, for example, an immune checkpoint inhibitor pathway receptor (e.g., PD-1). Precursor cells of immune cells that can be used according to the invention, include, hematopoietic stem and/or progenitor cells. Hematopoietic stem and/or progenitor cells can be derived from bone marrow, umbilical cord blood, adult peripheral blood after cytokine mobilization, and the like, by methods known in the art. The immune cells are engineered to recombinantly express the CARs of the invention.

Immune cells and precursor cells thereof can be isolated by methods known in the art, including commercially available methods (see, e.g., Rowland Jones et al., Lymphocytes: A Practical Approach, Oxford University Press, NY 1999). Sources for immune cells or precursors thereof include, but are not limited to, peripheral blood, umbilical cord blood, bone marrow, or other sources of hematopoietic cells. Various techniques can be employed to separate the cells to isolate or enrich desired immune cells. For instance, negative selection methods can be used to remove cells that are not the desired immune cells. Additionally, positive selection methods can be used to isolate or enrich for the desired immune cells or precursors thereof, or a combination of positive and negative selection methods can be employed. If a particular type of cell is to be isolated, e.g., a particular T cell, various cell surface markers or combinations of markers (e.g., CD3, CD4, CD8, CD34) can be used to separate the cells.

The immune cells or precursor cells thereof can be autologous or non-autologous to the subject to which they are administered in the methods of treatment of the invention. Autologous cells are isolated from the subject to which the engineered immune cells recombinantly expressing the CAR are to be administered. Optionally, the cells can be obtained by leukapheresis, where leukocytes are selectively removed from withdrawn blood, made recombinant, and then retransfused into the donor. Alternatively, allogeneic cells from a non-autologous donor that is not the subject can be used. In the case of a non-autologous donor, the cells are typed and matched for human leukocyte antigen (HLA) to determine the appropriate level of compatibility. For both autologous and non-autologous cells, the cells can optionally be cryopreserved until ready for use.

Various methods for isolating immune cells that can be used for recombinant expression of the CARs of the invention have been described previously, and can be used, including, but not limited to, using peripheral donor lymphocytes (Sadelain et al., Nat. Rev. Cancer 2003; 3:35-45, Morgan et al., Science 2006; 314:126-9), using lymphocyte cultures derived from tumor infiltrating lymphocytes (TILs) in tumor biopsies (Panelli et al., J. Immunol. 2000; 164:495-504, Panelli et al., J. Immunol. 2000; 164:4382-92) and using selectively in vitro expanded antigen-specific peripheral blood leukocytes employing artificial antigen-presenting cells (AAPCs) or dendritic cells (Dupont et al., Cancer Res. 2005; 65:5417-427; Papanicolaou et al., Blood 2003; 102:2498-505). In the case of using stem cells, the cells can be isolated by methods well known in the art (see, e.g., Klug et al., Hematopoietic Stem Cell Protocols, Humana Press, NJ 2002; Freshney et al., Culture of Human Stem Cells, John Wiley & Sons 2007).

According to particular embodiments, the method of making the engineered immune cells comprises transfecting or transducing immune effector cells isolated from an individual such that the immune effector cells express one or more CAR(s) according to embodiments of the invention. Methods of preparing immune cells for immunotherapy are described, e.g., in WO2014/130635, WO2013/176916 and WO2013/176915, which are incorporated herein by reference. Individual steps that can be used for preparing engineered immune cells are disclosed, e.g., in WO2014/039523, WO2014/184741, WO2014/191128, WO2014/184744 and WO2014/184143, which are incorporated herein by reference.

In a particular embodiment, the immune effector cells, such as T cells, are genetically modified with CARs of the invention (e.g., transduced with a viral vector comprising a nucleic acid encoding a CAR) and then are activated and expanded in vitro. In various embodiments, T cells can be activated and expanded before or after genetic modification to express a CAR, using methods as described, for example, in U.S. Pat. Nos. 6,352,694, 6,534,055, 6,905,680, 6,692,964, 5,858,358, 6,887,466, 6,905,681, 7,144,575, 7,067,318, 7,172,869, 7,232,566, 7,175,843, 5,883,223, 6,905,874, 6,797,514, 6,867,041, US2006/121005, which are incorporated herein by reference. T cells can be expanded in vitro or in vivo. Generally, the T cells of the invention can be expanded by contact with a surface having attached thereto an agent that stimulates a CD3/TCR complex-associated signal and a ligand that stimulates a co-stimulatory molecule on the surface of the T cells. As non-limiting examples, T cell populations can be stimulated as described herein, such as by contact with an anti-CD3 antibody, or antigen-binding fragment thereof, or an anti-CD3 antibody immobilized on a surface, or by contact with a protein kinase C activator (e.g., bryostatin) in conjunction with a calcium ionophore, or by activation of the CAR itself. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. Conditions appropriate for T cell culture include, e.g., an appropriate media (e.g., Minimal Essential Media or RPMI Media 1640 or, X-vivo 5 (Lonza)) that can contain factors necessary for proliferation and viability, including serum (e.g., fetal bovine or human serum), cytokines, such as IL-2, IL-7, IL-15, and/or IL-21, insulin, IFN-g, GM-CSF, TGFβ and/or any other additives for the growth of cells known to the skilled artisan. In other embodiments, the T cells can be activated and stimulated to proliferate with feeder cells and appropriate antibodies and cytokines using methods such as those described in U.S. Pat. Nos. 6,040,177, 5,827,642, and WO2012129514, which are incorporated herein by reference.

### Antibodies and Antigen Binding Domains

As used herein, the term “antibody” is used in a broad sense and includes immunoglobulin or antibody molecules including human, humanized, composite and chimeric antibodies and antibody fragments that are monoclonal or polyclonal. In general, antibodies are proteins or peptide chains that exhibit binding specificity to a specific antigen. Antibody structures are well known. Immunoglobulins can be assigned to five major classes (i.e., IgA, IgD, IgE, IgG and IgM), depending on the heavy chain constant domain amino acid sequence. IgA and IgG are further sub-classified as the isotypes IgA1, IgA2, IgG1, IgG2, IgG3 and IgG4. Accordingly, the antibodies of the invention can be of any of the five major classes or corresponding sub-classes. Preferably, the antibodies of the invention are IgG1, IgG2, IgG3 or IgG4. Antibody light chains of vertebrate species can be assigned to one of two clearly distinct types, namely kappa and lambda, based on the amino acid sequences of their constant domains. Accordingly, the antibodies of the invention can contain a kappa or lambda light chain constant domain. According to particular embodiments, the antibodies of the invention include heavy and/or light chain constant regions from rat or human antibodies. In addition to the heavy and light constant domains, antibodies contain an antigen-binding region that is made up of a light chain variable region and a heavy chain variable region, each of which contains three domains (i.e., complementarity determining regions 1-3; CDR1, CDR2, and CDR3). The light chain variable region domains are alternatively referred to as LCDR1, LCDR2, and LCDR3, and the heavy chain variable region domains are alternatively referred to as HCDR1, HCDR2, and HCDR3.

As used herein, the term an “isolated antibody” refers to an antibody which is substantially free of other antibodies having different antigenic specificities (e.g., an isolated antibody that specifically binds to DLL3 is substantially free of antibodies that do not bind to DLL3). In addition, an isolated antibody is substantially free of other cellular material and/or chemicals.

As used herein, the term “monoclonal antibody” refers to an antibody obtained from a population of substantially homogeneous antibodies, i.e., the individual antibodies comprising the population are identical except for possible naturally occurring mutations that may be present in minor amounts. The monoclonal antibodies of the invention can be made by the hybridoma method, phage display technology, single lymphocyte gene cloning technology, or by recombinant DNA methods. For example, the monoclonal antibodies can be produced by a hybridoma which includes a B cell obtained from a transgenic nonhuman animal, such as a transgenic mouse or rat, having a genome comprising a human heavy chain transgene and a light chain transgene.

As used herein, the term “antigen-binding fragment” and/or “antigen binding domain” refers to an antibody fragment such as, for example, a diabody, a Fab, a Fab′, a F(ab′)2, an Fv fragment, a disulfide stabilized Fv fragment (dsFv), a (dsFv)2, a bispecific dsFv (dsFv-dsFv′), a disulfide stabilized diabody (ds diabody), a single-chain antibody molecule (scFv), a single domain antibody (sdab) an scFv dimer (bivalent diabody), a multispecific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure. An antigen binding domain is capable of binding to the same antigen to which the parent antibody binds. According to particular embodiments, the antigen binding domain comprises a single-chain antibody molecule (scFv).

As used herein, the term “single-chain antibody” refers to a conventional single-chain antibody in the field, which comprises a heavy chain variable region and a light chain variable region connected by a short peptide of about 5 to about 20 amino acids. As used herein, the term “single domain antibody” refers to a conventional single domain antibody in the field, which comprises a heavy chain variable region and a heavy chain constant region or which comprises only a heavy chain variable region.

As used herein, the term “human antibody” refers to an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide.

As used herein, the term “humanized antibody” and/or “humanized antigen binding domain” refers to a non-human antibody and/or non-human antigen binding domain that is modified to increase the sequence homology to that of a human antibody and/or a human antigen binding domain, such that the antigen-binding properties of the antigen binding domain are retained, but its antigenicity in the human body is reduced.

As used herein, the term “chimeric antibody” and/or “chimeric antigen binding domain” refers to an antibody and/or antigen binding domain wherein the amino acid sequence of the immunoglobulin molecule is derived from two or more species. The variable region of both the light and heavy chains often corresponds to the variable region of an antibody and/or antigen binding domain derived from one species of mammal (e.g., mouse, rat, rabbit, etc.) having the desired specificity, affinity, and capability, while the constant regions correspond to the sequences of an antibody and/or antigen binding domain derived from another species of mammal (e.g., human) to avoid eliciting an immune response in that species.

As used herein, the term “multispecific antibody” refers to an antibody that comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, the first and second epitopes are on the same antigen, e.g., the same protein (or subunit of a multimeric protein). In an embodiment, the first and second epitopes overlap or substantially overlap. In an embodiment, the first and second epitopes do not overlap or do not substantially overlap. In an embodiment, the first and second epitopes are on different antigens, e.g., the different proteins (or different subunits of a multimeric protein). In an embodiment, a multispecific antibody comprises a third, fourth, or fifth immunoglobulin variable domain. In an embodiment, a multispecific antibody is a bispecific antibody molecule, a trispecific antibody molecule, or a tetraspecific antibody molecule.

As used herein, the term “bispecifc antibody” refers to a multispecific antibody that binds no more than two epitopes or two antigens. A bispecific antibody is characterized by a first immunoglobulin variable domain sequence which has binding specificity for a first epitope and a second immunoglobulin variable domain sequence that has binding specificity for a second epitope. In an embodiment, the first and second epitopes are on the same antigen, e.g., the same protein (or subunit of a multimeric protein). In an embodiment, the first and second epitopes overlap or substantially overlap. In an embodiment, the first and second epitopes are on different antigens, e.g., the different proteins (or different subunits of a multimeric protein). In an embodiment, a bispecific antibody comprises a heavy chain variable domain sequence and a light chain variable domain sequence which have binding specificity for a first epitope and a heavy chain variable domain sequence and a light chain variable domain sequence which have binding specificity for a second epitope. In an embodiment, a bispecific antibody comprises a half antibody, or fragment thereof, having binding specificity for a first epitope and a half antibody, or fragment thereof, having binding specificity for a second epitope. In an embodiment, a bispecific antibody comprises a scFv, or fragment thereof, having binding specificity for a first epitope, and a scFv, or fragment thereof, having binding specificity for a second epitope. In an embodiment, the first epitope is located on DLL3 and the second epitope is located on PD-1, PD-L1, TIM-3, LAG-3, CD73, apelin, CTLA-4, EGFR, HER-2, CD3, CD19, CD20, CD33, CD47, TIP-1, CLDN18.2, FOLR1, and/or other tumor associated immune suppressors or surface antigens.

As used herein, the term “DLL3” refers to Delta like canonical Notch ligand 3 (DLL3), also known as delta like 3 or delta like protein 3, is required for somite segmentation during early development (Dunwoodie et al., Development 2002; 129:1795-806). Unlike the mammalian Notch family ligands DLL1, DLL4, JAG1, and JAG2, which all activate Notch receptor signaling in trans (Ntziachristos et al., Cancer Cell 2014; 25(3):318-34), DLL3 is predominantly localized in the Golgi apparatus and is unable to activate Notch signaling (Chapman et al., Hum Mol Genet 2011; 20(5):905-16 and Geffers et al., J Cell Biol 2007; 178(3):465-76). During normal development, DLL3 inhibits both cis- and trans-acting Notch pathway activation by interacting with Notch and DLL1 (Chapman et al., Hum Mol Genet 2011; 20(5):905-16). DLL3 is normally either absent or present at very low levels in adult normal tissues except brain, but is overexpressed in lung cancer, testicular cancer, glioma and melanoma samples (Uhlen et al., Science 2017; 357(6352):eaan2507). Further, DLL3 is detectable on the surface of small cell lung cancer (SCLC) and large cell neuroendocrine carcinoma (LCNEC) tumor cells (Saunders et al., Sci Transl Med 2015; 7(302):302ra136 and Sharma et al., Cancer Res 2017; 77(14):3931-3941), making it a potential target of monoclonal antibodies and/or chimeric antigen receptors (CARs) for cancer therapy. The term “human DLL3” refers to a DLL3 originated from a human. An exemplary amino acid sequence of a human DLL3 is represented in GenBank Accession No. NP_058637.1 (SEQ ID NO:169).

As used herein, an antibody and/or antigen binding domain that “specifically binds to DLL3” refers to an antibody and/or antigen binding domain that binds to a DLL3, preferably a human DLL3, with a KD of 1×10⁻⁷ M or less, preferably 1×10⁻⁸ M or less, more preferably 5×10⁻⁹ M or less, 1×10⁻⁹ M or less, 5×10⁻¹⁰ M or less, or 1×10⁻¹⁰ M or less. The term “KD” refers to the dissociation constant, which is obtained from the ratio of Kd to Ka (i.e., Kd/Ka) and is expressed as a molar concentration (M). KD values for antigen binding domains can be determined using methods in the art in view of the present disclosure. For example, the KD of an antibody and/or antigen binding domain can be determined by using surface plasmon resonance, such as by using a biosensor system, e.g., a Biacore® system, or by using bio-layer interferometry technology, such as an Octet RED96 system.

The smaller the value of the KD of an antibody and/or antigen binding domain, the higher affinity that the antibody and/or antigen binding domain binds to a target antigen.

According to a particular aspect, the invention relates to chimeric antigen receptors (CAR)s comprising an antigen binding domain, wherein the antigen binding domain comprises a heavy chain complementarity determining region 1 (HCDR1), HCDR2, HCDR3, a light chain complementarity determining region 1 (LCDR1), LCDR2, and LCDR3, having the polypeptide sequences of:
(1) SEQ ID NOs: 25, 26, 27, 61, 62 and 63, respectively;
(2) SEQ ID NOs: 28, 29, 30, 64, 65 and 66, respectively;
(3) SEQ ID NOs: 31, 32, 33, 67, 68 and 69, respectively;
(4) SEQ ID NOs: 34, 35, 36, 70, 71 and 72, respectively;
(5) SEQ ID NOs: 37, 38, 39, 73, 74 and 75, respectively;
(6) SEQ ID NOs: 40, 41, 42, 76, 77 and 78, respectively;
(7) SEQ ID NOs: 43, 44, 45, 79, 80 and 81, respectively;
(8) SEQ ID NOs: 46, 47, 48, 82, 83 and 84, respectively;
(9) SEQ ID NOs: 49, 50, 51, 85, 86 and 87, respectively;
(10) SEQ ID NOs: 52, 53, 54, 88, 89 and 90, respectively;
(11) SEQ ID NOs: 55, 56, 57, 91, 92 and 93, respectively; or
(12) SEQ ID NOs: 58, 59, 60, 94, 95 and 96, respectively;
   wherein the antigen binding domain specifically binds DLL3, preferably human DLL3.

According to another particular aspect, the invention relates to chimeric antigen receptors (CARs) comprising an antigen binding domain, wherein the antigen binding domain comprises a heavy chain complementarity determining region 1 (HCDR1), HCDR2, HCDR3, a light chain complementarity determining region 1 (LCDR1), LCDR2, and LCDR3, having the polypeptide sequences of:
(1) SEQ ID NOs: 97, 98, 99, 133, 134 and 135, respectively;
(2) SEQ ID NOs: 100, 101, 102, 136, 137 and 138, respectively;
(3) SEQ ID NOs: 103, 104, 105, 139, 140 and 141, respectively;
(4) SEQ ID NOs: 106, 107, 108, 142, 143 and 144, respectively;
(5) SEQ ID NOs: 109, 110, 111, 145, 146 and 147, respectively;
(6) SEQ ID NOs: 112, 113, 114, 148, 149 and 150, respectively;
(7) SEQ ID NOs: 115, 116, 117, 151, 152 and 153, respectively;
(8) SEQ ID NOs: 118, 119, 120, 154, 155 and 156, respectively;
(9) SEQ ID NOs: 121, 122, 123, 157, 158 and 159, respectively;
(10) SEQ ID NOs: 124, 125, 126, 160, 161 and 162, respectively;
(11) SEQ ID NOs: 127, 128, 129, 163, 164 and 165, respectively; or
(12) SEQ ID NOs: 130, 131, 132, 166, 167 and 168, respectively;
   wherein the antigen binding domain specifically binds DLL3, preferably human DLL3.

According to another particular aspect, the invention relates to an antigen binding domain comprising a heavy chain variable region having a polypeptide sequence at least 95%, at least 96%, at least 97%, at least 98%, or at least 99% identical to SEQ ID NO: 1, 3, 5, 7, 9, 11, 13, 15, 17, 19, 21, or 23, or a light chain variable region having a polypeptide sequence at least 95%, at least 96%, at least 97%, at least 98%, or at least 99% identical to SEQ ID NO: 2, 4, 6, 8, 10, 12, 14, 16, 18, 20, 22, or 24.

According to another particular aspect, the invention relates to an antigen binding domain, comprising:
(1) a heavy chain variable region having the polypeptide sequence of SEQ ID NO:1, and a light chain variable region having the polypeptide sequence of SEQ ID NO:2;
(2) a heavy chain variable region having the polypeptide sequence of SEQ ID NO:3, and a light chain variable region having the polypeptide sequence of SEQ ID NO:4;
(3) a heavy chain variable region having the polypeptide sequence of SEQ ID NO:5, and a light chain variable region having the polypeptide sequence of SEQ ID NO:6;
(4) a heavy chain variable region having the polypeptide sequence of SEQ ID NO:7, and a light chain variable region having the polypeptide sequence of SEQ ID NO:8;
(5) a heavy chain variable region having the polypeptide sequence of SEQ ID NO:9, and a light chain variable region having the polypeptide sequence of SEQ ID NO:10;
(6) a heavy chain variable region having the polypeptide sequence of SEQ ID NO:11, and a light chain variable region having the polypeptide sequence of SEQ ID NO:12;
(7) a heavy chain variable region having the polypeptide sequence of SEQ ID NO:13, and a light chain variable region having the polypeptide sequence of SEQ ID NO:14;
(8) a heavy chain variable region having the polypeptide sequence of SEQ ID NO:15, and a light chain variable region having the polypeptide sequence of SEQ ID NO:16;
(9) a heavy chain variable region having the polypeptide sequence of SEQ ID NO:17, and a light chain variable region having the polypeptide sequence of SEQ ID NO:18;
(10) a heavy chain variable region having the polypeptide sequence of SEQ ID NO:19, and a light chain variable region having the polypeptide sequence of SEQ ID NO:20;
(11) a heavy chain variable region having the polypeptide sequence of SEQ ID NO:21, and a light chain variable region having the polypeptide sequence of SEQ ID NO:22; or
(12) a heavy chain variable region having the polypeptide sequence of SEQ ID NO:23, and a light chain variable region having the polypeptide sequence of SEQ ID NO:24.

According to another particular aspect, the antigen binding domain is humanized and comprises a heavy chain variable region having a polypeptide sequence at least 95%, at least 96%, at least 97%, at least 98%, or at least 99% identical to any one of SEQ ID NOs: 170, 175-209 or 248-255, or a light chain variable region having a polypeptide sequence at least 95%, at least 96%, at least 97%, at least 98%, or at least 99% identical to any one of SEQ ID NOs: 171-174, 210-240 or 256-264.

According to another particular aspect, the antigen binding domain is humanized and comprises:
(1) a heavy chain variable region having the polypeptide sequence of SEQ ID NO:170, and a light chain variable region having the polypeptide sequence of SEQ ID NO:171;
(2) a heavy chain variable region having the polypeptide sequence of SEQ ID NO:170, and a light chain variable region having the polypeptide sequence of SEQ ID NO:172;
(3) a heavy chain variable region having the polypeptide sequence of SEQ ID NO:170, and a light chain variable region having the polypeptide sequence of SEQ ID NO:173;
(4) a heavy chain variable region having the polypeptide sequence of SEQ ID NO:183, and a light chain variable region having the polypeptide sequence of SEQ ID NO:217;
(5) a heavy chain variable region having the polypeptide sequence of SEQ ID NO:183, and a light chain variable region having the polypeptide sequence of SEQ ID NO:218;
(6) a heavy chain variable region having the polypeptide sequence of SEQ ID NO:184, and a light chain variable region having the polypeptide sequence of SEQ ID NO:217;
(7) a heavy chain variable region having the polypeptide sequence of SEQ ID NO:184, and a light chain variable region having the polypeptide sequence of SEQ ID NO:218;
(8) a heavy chain variable region having the polypeptide sequence of SEQ ID NO:198, and a light chain variable region having the polypeptide sequence of SEQ ID NO:229;
(9) a heavy chain variable region having the polypeptide sequence of SEQ ID NO:200, and a light chain variable region having the polypeptide sequence of SEQ ID NO:229;
(10) a heavy chain variable region having the polypeptide sequence of SEQ ID NO:198, and a light chain variable region having the polypeptide sequence of SEQ ID NO:231;
(11) a heavy chain variable region having the polypeptide sequence of SEQ ID NO:200, and a light chain variable region having the polypeptide sequence of SEQ ID NO:231;
(12) a heavy chain variable region having the polypeptide sequence of SEQ ID NO:201, and a light chain variable region having the polypeptide sequence of SEQ ID NO:229;
(13) a heavy chain variable region having the polypeptide sequence of SEQ ID NO:201, and a light chain variable region having the polypeptide sequence of SEQ ID NO:230;
(14) a heavy chain variable region having the polypeptide sequence of SEQ ID NO:201, and a light chain variable region having the polypeptide sequence of SEQ ID NO:231;
(15) a heavy chain variable region having the polypeptide sequence of SEQ ID NO:175, and a light chain variable region having the polypeptide sequence of SEQ ID NO:210;
(16) a heavy chain variable region having the polypeptide sequence of SEQ ID NO:175, and a light chain variable region having the polypeptide sequence of SEQ ID NO:211;
(17) a heavy chain variable region having the polypeptide sequence of SEQ ID NO:175, and a light chain variable region having the polypeptide sequence of SEQ ID NO:212;
(18) a heavy chain variable region having the polypeptide sequence of SEQ ID NO:176, and a light chain variable region having the polypeptide sequence of SEQ ID NO:210;
(19) a heavy chain variable region having the polypeptide sequence of SEQ ID NO:176, and a light chain variable region having the polypeptide sequence of SEQ ID NO:211;
(20) a heavy chain variable region having the polypeptide sequence of SEQ ID NO:176, and a light chain variable region having the polypeptide sequence of SEQ ID NO:212;
(21) a heavy chain variable region having the polypeptide sequence of SEQ ID NO:177, and a light chain variable region having the polypeptide sequence of SEQ ID NO:210;
(22) a heavy chain variable region having the polypeptide sequence of SEQ ID NO:177, and a light chain variable region having the polypeptide sequence of SEQ ID NO:211;
(23) a heavy chain variable region having the polypeptide sequence of SEQ ID NO:178, and a light chain variable region having the polypeptide sequence of SEQ ID NO:210;
(24) a heavy chain variable region having the polypeptide sequence of SEQ ID NO:178, and a light chain variable region having the polypeptide sequence of SEQ ID NO:211;
(25) a heavy chain variable region having the polypeptide sequence of SEQ ID NO:177, and a light chain variable region having the polypeptide sequence of SEQ ID NO:211;
(26) a heavy chain variable region having the polypeptide sequence of SEQ ID NO:177, and a light chain variable region having the polypeptide sequence of SEQ ID NO:212;
(27) a heavy chain variable region having the polypeptide sequence of SEQ ID NO:178, and a light chain variable region having the polypeptide sequence of SEQ ID NO:212;
(28) a heavy chain variable region having the polypeptide sequence of SEQ ID NO:179, and a light chain variable region having the polypeptide sequence of SEQ ID NO:213;
(29) a heavy chain variable region having the polypeptide sequence of SEQ ID NO:179, and a light chain variable region having the polypeptide sequence of SEQ ID NO:214;
(30) a heavy chain variable region having the polypeptide sequence of SEQ ID NO:179, and a light chain variable region having the polypeptide sequence of SEQ ID NO:215;
(31) a heavy chain variable region having the polypeptide sequence of SEQ ID NO:180, and a light chain variable region having the polypeptide sequence of SEQ ID NO:213;
(32) a heavy chain variable region having the polypeptide sequence of SEQ ID NO:180, and a light chain variable region having the polypeptide sequence of SEQ ID NO:214;
(33) a heavy chain variable region having the polypeptide sequence of SEQ ID NO:180, and a light chain variable region having the polypeptide sequence of SEQ ID NO:215;
(34) a heavy chain variable region having the polypeptide sequence of SEQ ID NO:181, and a light chain variable region having the polypeptide sequence of SEQ ID NO:213;
(35) a heavy chain variable region having the polypeptide sequence of SEQ ID NO:181, and a light chain variable region having the polypeptide sequence of SEQ ID NO:214;
(36) a heavy chain variable region having the polypeptide sequence of SEQ ID NO:182, and a light chain variable region having the polypeptide sequence of SEQ ID NO:215;
(37) a heavy chain variable region having the polypeptide sequence of SEQ ID NO:202, and a light chain variable region having the polypeptide sequence of SEQ ID NO:232;
(38) a heavy chain variable region having the polypeptide sequence of SEQ ID NO:202, and a light chain variable region having the polypeptide sequence of SEQ ID NO:233;
(39) a heavy chain variable region having the polypeptide sequence of SEQ ID NO:202, and a light chain variable region having the polypeptide sequence of SEQ ID NO:234;
(40) a heavy chain variable region having the polypeptide sequence of SEQ ID NO:203, and a light chain variable region having the polypeptide sequence of SEQ ID NO:232;
(41) a heavy chain variable region having the polypeptide sequence of SEQ ID NO:203, and a light chain variable region having the polypeptide sequence of SEQ ID NO:233;
(42) a heavy chain variable region having the polypeptide sequence of SEQ ID NO:203, and a light chain variable region having the polypeptide sequence of SEQ ID NO:234;
(43) a heavy chain variable region having the polypeptide sequence of SEQ ID NO:204, and a light chain variable region having the polypeptide sequence of SEQ ID NO:234;
(44) a heavy chain variable region having the polypeptide sequence of SEQ ID NO:208, and a light chain variable region having the polypeptide sequence of SEQ ID NO:239;
(45) a heavy chain variable region having the polypeptide sequence of SEQ ID NO:208, and a light chain variable region having the polypeptide sequence of SEQ ID NO:240;
(46) a heavy chain variable region having the polypeptide sequence of SEQ ID NO:253, and a light chain variable region having the polypeptide sequence of SEQ ID NO:261; or
(47) a heavy chain variable region having the polypeptide sequence of SEQ ID NO:255, and a light chain variable region having the polypeptide sequence of SEQ ID NO:263.

According to another particular aspect, the antigen binding domain is a single chain variable fragment (scFv) that specifically binds DLL3, preferably human DLL3. In certain embodiments, the antigen binding domain is a humanized single chain variable fragment (scFv) that specifically binds DLL3, preferably human DLL3.

In certain embodiments, the single chain variable fragment (scFv) comprises a polypeptide sequence at least 95%, at least 96%, at least 97%, at least 98%, or at least 99% identical to any one of SEQ ID NOs:241-247 or 265-286. In certain embodiments, the single chain variable fragment (scFv) comprises a polypeptide sequence having an amino acid sequence selected from the group consisting of SEQ ID NOs:241-247 or 265-286.

According to another particular aspect, the chimeric antigen receptor comprises one or more antigen binding domains.

According to another particular aspect, the intracellular signaling domain comprises one or more costimulatory domains and one or more activating domains.

In another general aspect, the invention relates to an isolated polynucleotide comprising a nucleic acid encoding chimeric antigen receptor (CAR), wherein the CAR comprises an antigen binding domain thereof of the invention. It will be appreciated by those skilled in the art that the coding sequence of a protein can be changed (e.g., replaced, deleted, inserted, etc.) without changing the amino acid sequence of the protein. Accordingly, it will be understood by those skilled in the art that nucleic acid sequences encoding antigen binding domains thereof of the invention can be altered without changing the amino acid sequences of the proteins.

In another general aspect, the invention relates to a vector comprising the isolated polynucleotide comprising the nucleic acid encoding the CAR, wherein the CAR comprises an antigen binding domain thereof of the invention. Any vector known to those skilled in the art in view of the present disclosure can be used, such as a plasmid, a cosmid, a phage vector or a viral vector. In some embodiments, the vector is a recombinant expression vector such as a plasmid. The vector can include any element to establish a conventional function of an expression vector, for example, a promoter, ribosome binding element, terminator, enhancer, selection marker, and origin of replication. The promoter can be a constitutive, inducible, or repressible promoter. A number of expression vectors capable of delivering nucleic acids to a cell are known in the art and can be used herein for production of an antigen binding domain thereof in the cell. Conventional cloning techniques or artificial gene synthesis can be used to generate a recombinant expression vector according to embodiments of the invention.

In another general aspect, the invention relates to a cell transduced with the vector comprising the isolated nucleic acids encoding the CARs of the invention. The term “transduced” or “transduction” refers to a process by which exogenous nucleic acid is transferred or introduced into the host cell. A “transduced” cell is one which has been transduced with exogenous nucleic acid. The cell includes the primary subject cell and its progeny. In certain embodiments, the cell is a human CAR-T cell, wherein the T cell is engineered to express the CAR of the invention to treat diseases such as cancer. In certain embodiments, the cell is a human CAR-NK cell, wherein the NK cell engineered to express the CAR of the invention is used to treat diseases such as cancer.

In another general aspect, the invention relates to a method of making a CAR-T cell by transducing a T cell with a vector comprising the isolated nucleic acids encoding the CARs of the invention.

In another general aspect, the invention relates to a method of producing the CAR-T cell thereof of the invention, comprising culturing T cells comprising a nucleic acid encoding a chimeric antigen receptor (CAR) of the invention under conditions to produce the CAR-T cell, and recovering the CAR-T cell.

In another general aspect, the invention relates to a method of making a CAR-NK cell by transducing a NK cell with a vector comprising the isolated nucleic acids encoding the CARs of the invention.

In another general aspect, the invention relates to a method of producing a CAR-NK cell of the invention, comprising culturing NK cells comprising nucleic acids encoding the chimeric antigen receptor (CAR) thereof under conditions to produce the CAR-NK cell, and recovering the CAR-NK cell.

In another general aspect, the invention relates to a method of generating a population of RNA-engineered cells comprising a chimeric antigen receptor (CAR) of the invention. The methods comprise contacting a population of cells with isolated polynucleotides comprising a nucleic acid encoding a CAR of the invention, wherein the isolated polynucleotides are in vitro transcribed RNA or synthetic RNA.

In another general aspect, the invention relates to a humanized anti-DLL3 monoclonal antibody or antigen-binding fragment thereof, wherein the antibody or antigen-binding fragment thereof comprise a heavy chain variable region having a polypeptide sequence at least 95% identical to any one of SEQ ID NOs: 170, 175-209 or 248-255, or a light chain variable region having a polypeptide sequence at least 95% identical to any one of SEQ ID NOs: 171-174, 210-240 or 256-264.

According to another particular aspect, the humanized anti-DLL3 monoclonal antibodies or antigen-binding fragments thereof comprise:
(1) a heavy chain variable region having the polypeptide sequence of SEQ ID NO:170, and a light chain variable region having the polypeptide sequence of SEQ ID NO:171;
(2) a heavy chain variable region having the polypeptide sequence of SEQ ID NO:170, and a light chain variable region having the polypeptide sequence of SEQ ID NO:172;
(3) a heavy chain variable region having the polypeptide sequence of SEQ ID NO:170, and a light chain variable region having the polypeptide sequence of SEQ ID NO:173.
(4) a heavy chain variable region having the polypeptide sequence of SEQ ID NO:183, and a light chain variable region having the polypeptide sequence of SEQ ID NO:217;
(5) a heavy chain variable region having the polypeptide sequence of SEQ ID NO:183, and a light chain variable region having the polypeptide sequence of SEQ ID NO:218;
(6) a heavy chain variable region having the polypeptide sequence of SEQ ID NO:184, and a light chain variable region having the polypeptide sequence of SEQ ID NO:217;
(7) a heavy chain variable region having the polypeptide sequence of SEQ ID NO:184, and a light chain variable region having the polypeptide sequence of SEQ ID NO:218;
(8) a heavy chain variable region having the polypeptide sequence of SEQ ID NO:198, and a light chain variable region having the polypeptide sequence of SEQ ID NO:229;
(9) a heavy chain variable region having the polypeptide sequence of SEQ ID NO:200, and a light chain variable region having the polypeptide sequence of SEQ ID NO:229;
(10) a heavy chain variable region having the polypeptide sequence of SEQ ID NO:198, and a light chain variable region having the polypeptide sequence of SEQ ID NO:231;
(11) a heavy chain variable region having the polypeptide sequence of SEQ ID NO:200, and a light chain variable region having the polypeptide sequence of SEQ ID NO:231;
(12) a heavy chain variable region having the polypeptide sequence of SEQ ID NO:201, and a light chain variable region having the polypeptide sequence of SEQ ID NO:229;
(13) a heavy chain variable region having the polypeptide sequence of SEQ ID NO:201, and a light chain variable region having the polypeptide sequence of SEQ ID NO:230;
(14) a heavy chain variable region having the polypeptide sequence of SEQ ID NO:201, and a light chain variable region having the polypeptide sequence of SEQ ID NO:231;
(15) a heavy chain variable region having the polypeptide sequence of SEQ ID NO:175, and a light chain variable region having the polypeptide sequence of SEQ ID NO:210;
(16) a heavy chain variable region having the polypeptide sequence of SEQ ID NO:175, and a light chain variable region having the polypeptide sequence of SEQ ID NO:211;
(17) a heavy chain variable region having the polypeptide sequence of SEQ ID NO:175, and a light chain variable region having the polypeptide sequence of SEQ ID NO:212;
(18) a heavy chain variable region having the polypeptide sequence of SEQ ID NO:176, and a light chain variable region having the polypeptide sequence of SEQ ID NO:210;
(19) a heavy chain variable region having the polypeptide sequence of SEQ ID NO:176, and a light chain variable region having the polypeptide sequence of SEQ ID NO:211;
(20) a heavy chain variable region having the polypeptide sequence of SEQ ID NO:176, and a light chain variable region having the polypeptide sequence of SEQ ID NO:212;
(21) a heavy chain variable region having the polypeptide sequence of SEQ ID NO:177, and a light chain variable region having the polypeptide sequence of SEQ ID NO:210;
(22) a heavy chain variable region having the polypeptide sequence of SEQ ID NO:177, and a light chain variable region having the polypeptide sequence of SEQ ID NO:211;
(23) a heavy chain variable region having the polypeptide sequence of SEQ ID NO:178, and a light chain variable region having the polypeptide sequence of SEQ ID NO:210;
(24) a heavy chain variable region having the polypeptide sequence of SEQ ID NO:178, and a light chain variable region having the polypeptide sequence of SEQ ID NO:211;
(25) a heavy chain variable region having the polypeptide sequence of SEQ ID NO:177, and a light chain variable region having the polypeptide sequence of SEQ ID NO:211;
(26) a heavy chain variable region having the polypeptide sequence of SEQ ID NO:177, and a light chain variable region having the polypeptide sequence of SEQ ID NO:212;
(27) a heavy chain variable region having the polypeptide sequence of SEQ ID NO:178, and a light chain variable region having the polypeptide sequence of SEQ ID NO:212;
(28) a heavy chain variable region having the polypeptide sequence of SEQ ID NO:179, and a light chain variable region having the polypeptide sequence of SEQ ID NO:213;
(29) a heavy chain variable region having the polypeptide sequence of SEQ ID NO:179, and a light chain variable region having the polypeptide sequence of SEQ ID NO:214;
(30) a heavy chain variable region having the polypeptide sequence of SEQ ID NO:179, and a light chain variable region having the polypeptide sequence of SEQ ID NO:215;
(31) a heavy chain variable region having the polypeptide sequence of SEQ ID NO:180, and a light chain variable region having the polypeptide sequence of SEQ ID NO:213;
(32) a heavy chain variable region having the polypeptide sequence of SEQ ID NO:180, and a light chain variable region having the polypeptide sequence of SEQ ID NO:214;
(33) a heavy chain variable region having the polypeptide sequence of SEQ ID NO:180, and a light chain variable region having the polypeptide sequence of SEQ ID NO:215;
(34) a heavy chain variable region having the polypeptide sequence of SEQ ID NO:181, and a light chain variable region having the polypeptide sequence of SEQ ID NO:213;
(35) a heavy chain variable region having the polypeptide sequence of SEQ ID NO:181, and a light chain variable region having the polypeptide sequence of SEQ ID NO:214;
(36) a heavy chain variable region having the polypeptide sequence of SEQ ID NO:182, and a light chain variable region having the polypeptide sequence of SEQ ID NO:215;
(37) a heavy chain variable region having the polypeptide sequence of SEQ ID NO:202, and a light chain variable region having the polypeptide sequence of SEQ ID NO:232;
(38) a heavy chain variable region having the polypeptide sequence of SEQ ID NO:202, and a light chain variable region having the polypeptide sequence of SEQ ID NO:233;
(39) a heavy chain variable region having the polypeptide sequence of SEQ ID NO:202, and a light chain variable region having the polypeptide sequence of SEQ ID NO:234;
(40) a heavy chain variable region having the polypeptide sequence of SEQ ID NO:203, and a light chain variable region having the polypeptide sequence of SEQ ID NO:232;
(41) a heavy chain variable region having the polypeptide sequence of SEQ ID NO:203, and a light chain variable region having the polypeptide sequence of SEQ ID NO:233;
(42) a heavy chain variable region having the polypeptide sequence of SEQ ID NO:203, and a light chain variable region having the polypeptide sequence of SEQ ID NO:234;
(43) a heavy chain variable region having the polypeptide sequence of SEQ ID NO:204, and a light chain variable region having the polypeptide sequence of SEQ ID NO:234;
(44) a heavy chain variable region having the polypeptide sequence of SEQ ID NO:208, and a light chain variable region having the polypeptide sequence of SEQ ID NO:239;
(45) a heavy chain variable region having the polypeptide sequence of SEQ ID NO:208, and a light chain variable region having the polypeptide sequence of SEQ ID NO:240;
(46) a heavy chain variable region having the polypeptide sequence of SEQ ID NO:253, and a light chain variable region having the polypeptide sequence of SEQ ID NO:261; or
(47) a heavy chain variable region having the polypeptide sequence of SEQ ID NO:255, and a light chain variable region having the polypeptide sequence of SEQ ID NO:263.

According to another particular aspect, the humanized anti-DLL3 monoclonal antibody or antigen-binding fragment thereof is capable of inducing effector-mediated tumor cell lysis, mediating the recruitment of conjugated drugs, and/or forms a bispecific antibody with another monoclonal antibody or antigen-binding fragment with a cancer-killing effect.

In another general aspect, the invention relates to an isolated nucleic acid encoding the humanized anti-DLL3 monoclonal antibodies or antigen-binding fragments thereof of the invention.

In another general aspect, the invention relates to a vector comprising the isolated nucleic acid encoding the humanized anti-DLL3 monoclonal antibodies or antigen-binding fragments thereof of the invention.

In another general aspect, the invention relates to a host cell comprising the vector comprising the isolated nucleic acid encoding the humanized anti-DLL3 monoclonal antibodies or antigen-binding fragments thereof of the invention.

In another general aspect, the invention relates to a method of producing the humanized anti-DLL3 monoclonal antibody or antigen-binding fragment thereof of the invention, comprising culturing a cell comprising a nucleic acid encoding the monoclonal antibody or antigen-binding fragment under conditions to produce the monoclonal antibody or antigen-binding fragment, and recovering the antibody or antigen-binding fragment from the cell or culture.

### Pharmaceutical Compositions

In another general aspect, the invention relates to a pharmaceutical composition comprising an isolated polynucleotide of the invention, an isolated polypeptide of the invention, a host cell of the invention, and/or an engineered immune cell of the invention and a pharmaceutically acceptable carrier.

In another general aspect, the invention relates to a pharmaceutical composition comprising a humanized anti-DLL3 monoclonal antibody or antigen-binding fragment thereof of the invention and a pharmaceutically acceptable carrier.

The term “pharmaceutical composition” as used herein means a product comprising an isolated polynucleotide of the invention, an isolated polypeptide of the invention, a host cell of the invention, and/or an engineered immune cell of the invention together with a pharmaceutically acceptable carrier. Polynucleotides, polypeptides, host cells, and/or engineered immune cells of the invention and compositions comprising them are also useful in the manufacture of a medicament for therapeutic applications mentioned herein.

As used herein, the term “carrier” refers to any excipient, diluent, filler, salt, buffer, stabilizer, solubilizer, oil, lipid, lipid containing vesicle, microsphere, liposomal encapsulation, or other material well known in the art for use in pharmaceutical formulations. It will be understood that the characteristics of the carrier, excipient or diluent will depend on the route of administration for a particular application. As used herein, the term “pharmaceutically acceptable carrier” refers to a non-toxic material that does not interfere with the effectiveness of a composition according to the invention or the biological activity of a composition according to the invention. According to particular embodiments, in view of the present disclosure, any pharmaceutically acceptable carrier suitable for use in a polynucleotide, polypeptide, host cell, and/or engineered immune cell pharmaceutical composition can be used in the invention.

The formulation of pharmaceutically active ingredients with pharmaceutically acceptable carriers is known in the art, e.g., Remington: The Science and Practice of Pharmacy (e.g. 21st edition (2005), and any later editions). Non-limiting examples of additional ingredients include: buffers, diluents, solvents, tonicity regulating agents, preservatives, stabilizers, and chelating agents. One or more pharmaceutically acceptable carriers may be used in formulating the pharmaceutical compositions of the invention.

In another general aspect, the invention relates to a method of producing a pharmaceutical composition comprising the humanized anti-DLL3 monoclonal antibody or antigen-binding fragment thereof of the invention, comprising combining the monoclonal antibody or antigen-binding fragment thereof with a pharmaceutically acceptable carrier to obtain the pharmaceutical composition.

### Methods of Use

In another general aspect, the invention relates to a method of treating a cancer in a subject in need thereof, comprising administering to the subject the CAR-T cells and/or CAR-NK cells of the invention. The cancer can be any liquid or solid cancer, for example, it can be selected from, but not limited to, a lung cancer such as small cell lung cancer (SCLC), large cell neuroendocrine carcinoma (LCNEC), a gastric cancer, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, and other solid tumors, and a non-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

In another general aspect, the invention relates to a method of targeting DLL3 on a cancer cell surface in a subject in need thereof, comprising administering to the subject in need thereof a pharmaceutical composition comprising the humanized anti-DLL3 monoclonal antibody or antigen-binding fragment thereof of the invention.

In another general aspect, the invention relates to a method of treating cancer in a subject in need thereof, comprising administering to the subject the pharmaceutical composition comprising the humanized anti-DLL3 monoclonal antibody or antigen-binding fragment thereof of the invention. The cancer can be any liquid or solid cancer, for example, it can be selected from, but not limited to, a lung cancer such as small cell lung cancer (SCLC), large cell neuroendocrine carcinoma (LCNEC), a gastric cancer, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, and other solid tumors, and a non-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

According to embodiments of the invention, the CAR-T cell or CAR-NK cells comprises a therapeutically effective amount of the expressed CARs of the invention and the pharmaceutical compositions comprise a “therapeutically effective amount” of the humanized anti-DLL monoclonal antibody or antigen-binding fragment thereof. As used herein, the term “therapeutically effective amount” refers to an amount of an active ingredient or component that elicits the desired biological or medicinal response in a subject. A therapeutically effective amount can be determined empirically and in a routine manner, in relation to the stated purpose.

As used herein with reference to CARs, a therapeutically effective amount means an amount of the CAR molecule expressed in the transduced T cell or NK cell that modulates an immune response in a subject in need thereof. Also, as used herein with reference to CARs, a therapeutically effective amount means an amount of the CAR molecule expressed in the transduced T cell or NK cell that results in treatment of a disease, disorder, or condition; prevents or slows the progression of the disease, disorder, or condition; or reduces or completely alleviates symptoms associated with the disease, disorder, or condition.

As used herein with reference to CAR-T cell or CAR-NK cell, a therapeutically effective amount means an amount of the CAR-T cells or CAR-NK cells that modulates an immune response in a subject in need thereof. Also, as used herein with reference to CAR-T cell or CAR-NK cell, a therapeutically effective amount means an amount of the CAR-T cells or CAR-NK cells that results in treatment of a disease, disorder, or condition; prevents or slows the progression of the disease, disorder, or condition; or reduces or completely alleviates symptoms associated with the disease, disorder, or condition.

As used herein with reference to a humanized anti-DLL3 monoclonal antibody or antigen-binding fragment thereof, a therapeutically effective amount means an amount of the humanized anti-DLL3 monoclonal antibody or antigen-binding fragment thereof that modulates an immune response in a subject in need thereof. Also, as used herein with reference to a humanized anti-DLL3 monoclonal antibody or antigen-binding fragment thereof, a therapeutically effective amount means an amount of the humanized anti-DLL3 monoclonal antibody or antigen binding fragment thereof that results in treatment of a disease, disorder, or condition; prevents or slows the progression of the disease, disorder, or condition; or reduces or completely alleviates symptoms associated with the disease, disorder, or condition.

According to particular embodiments, the disease, disorder or condition to be treated is cancer, preferably a cancer selected from the group consisting of a lung cancer such as small cell lung cancer (SCLC), large cell neuroendocrine carcinoma (LCNEC), a gastric cancer, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, and other solid tumors, and a non-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

According to particular embodiments, a therapeutically effective amount refers to the amount of therapy which is sufficient to achieve one, two, three, four, or more of the following effects: (i) reduce or ameliorate the severity of the disease, disorder or condition to be treated or a symptom associated therewith; (ii) reduce the duration of the disease, disorder or condition to be treated, or a symptom associated therewith; (iii) prevent the progression of the disease, disorder or condition to be treated, or a symptom associated therewith; (iv) cause regression of the disease, disorder or condition to be treated, or a symptom associated therewith; (v) prevent the development or onset of the disease, disorder or condition to be treated, or a symptom associated therewith; (vi) prevent the recurrence of the disease, disorder or condition to be treated, or a symptom associated therewith; (vii) reduce hospitalization of a subject having the disease, disorder or condition to be treated, or a symptom associated therewith; (viii) reduce hospitalization length of a subject having the disease, disorder or condition to be treated, or a symptom associated therewith; (ix) increase the survival of a subject with the disease, disorder or condition to be treated, or a symptom associated therewith; (xi) inhibit or reduce the disease, disorder or condition to be treated, or a symptom associated therewith in a subject; and/or (xii) enhance or improve the prophylactic or therapeutic effect(s) of another therapy.

The therapeutically effective amount or dosage can vary according to various factors, such as the disease, disorder or condition to be treated, the means of administration, the target site, the physiological state of the subject (including, e.g., age, body weight, health), whether the subject is a human or an animal, other medications administered, and whether the treatment is prophylactic or therapeutic. Treatment dosages are optimally titrated to optimize safety and efficacy.

According to particular embodiments, the compositions described herein are formulated to be suitable for the intended route of administration to a subject. For example, the compositions described herein can be formulated to be suitable for intravenous, subcutaneous, or intramuscular administration.

The cells of the invention can be administered in any convenient manner known to those skilled in the art. For example, the cells of the invention can be administered to the subject by aerosol inhalation, injection, ingestion, transfusion, implantation, and/or transplantation. The compositions comprising the cells of the invention can be administered transarterially, subcutaneously, intradermally, intratumorally, intranodally, intramedullary, intramuscularly, intrapleurally, by intravenous (i.v.) injection, or intraperitoneally. In certain embodiments, the cells of the invention can be administered with or without lymphodepletion of the subject.

The pharmaceutical compositions comprising cells of the invention expressing CARs of the invention can be provided in sterile liquid preparations, typically isotonic aqueous solutions with cell suspensions, or optionally as emulsions, dispersions, or the like, which are typically buffered to a selected pH. The compositions can comprise carriers, for example, water, saline, phosphate buffered saline, and the like, suitable for the integrity and viability of the cells, and for administration of a cell composition.

Sterile injectable solutions can be prepared by incorporating cells of the invention in a suitable amount of the appropriate solvent with various other ingredients, as desired. Such compositions can include a pharmaceutically acceptable carrier, diluent, or excipient such as sterile water, physiological saline, glucose, dextrose, or the like, that are suitable for use with a cell composition and for administration to a subject, such as a human. Suitable buffers for providing a cell composition are well known in the art. Any vehicle, diluent, or additive used is compatible with preserving the integrity and viability of the cells of the invention.

The cells of the invention can be administered in any physiologically acceptable vehicle. A cell population comprising cells of the invention can comprise a purified population of cells. Those skilled in the art can readily determine the cells in a cell population using various well known methods. The ranges in purity in cell populations comprising genetically modified cells of the invention can be from about 50% to about 55%, from about 55% to about 60%, from about 60% to about 65%, from about 65% to about 70%, from about 70% to about 75%, from about 75% to about 80%, from about 80% to about 85%, from about 85% to about 90%, from about 90% to about 95%, or from about 95% to about 100%. Dosages can be readily adjusted by those skilled in the art, for example, a decrease in purity could require an increase in dosage.

The cells of the invention are generally administered as a dose based on cells per kilogram (cells/kg) of body weight of the subject to which the cells are administered. Generally, the cell doses are in the range of about 10⁴ to about 10¹⁰ cells/kg of body weight, for example, about 10⁵ to about 10⁹, about 10⁵ to about 10⁸, about 10⁵ to about 10⁷, or about 10⁵ to about 10⁶, depending on the mode and location of administration. In general, in the case of systemic administration, a higher dose is used than in regional administration, where the immune cells of the invention are administered in the region of a tumor and/or cancer. Exemplary dose ranges include, but are not limited to, 1×10⁴ to 1×10⁸, 2×10⁴ to 1×10⁸, 3×10⁴ to 1×10⁸, 4×10⁴ to 1×10⁸, 5×10⁴ to 6×10⁸, 7×10⁴ to 1×10⁸, 8×10⁴ to 1×10⁸, 9×10⁴ to 1×10⁸, 1×10⁵ to 1×10⁸, 1×10⁵ to 9×10⁷, 1×10⁵ to 8×10⁷, 1×10⁵ to 7×10⁷, 1×10⁵ to 6×10⁷, 1×10⁵ to 5×10⁷, 1×10⁵ to 4×10⁷, 1×10⁵ to 4×10⁷, 1×10⁵ to 3×10⁷, 1×10⁵ to 2×10⁷, 1×10⁵ to 1×10⁷, 1×10⁵ to 9×10⁶, 1×10⁵ to 8×10⁶, 1×10⁵ to 7×10⁶, 1×10⁵ to 6×10⁶, 1×10⁵ to 5×10⁶, 1×10⁵ to 4×10⁶, 1×10⁵ to 4×10⁶, 1×10⁵ to 3×10⁶, 1×10⁵ to 2×10⁶, 1×10⁵ to 1×10⁶, 2×10⁵ to 9×10⁷, 2×10⁵ to 8×10⁷, 2×10⁵ to 7×10⁷, 2×10⁵ to 6×10⁷, 2×10⁵ to 5×10⁷, 2×10⁵ to 4×10⁷, 2×10⁵ to 4×10⁷, 2×10⁵ to 3×10⁷, 2×10⁵ to 2×10⁷, 2×10⁵ to 1×10⁷, 2×10⁵ to 9×10⁶, 2×10⁵ to 8×10⁶, 2×10⁵ to 7×10⁶, 2×10⁵ to 6×10⁶, 2×10⁵ to 5×10⁶, 2×10⁵ to 4×10⁶, 2×10⁵ to 4×10⁶, 2×10⁵ to 3×10⁶, 2×10⁵ to 2×10⁶, 2×10⁵ to 1×10⁶, 3×10⁵ to 3×10⁶ cells/kg, and the like. Additionally, the dose can be adjusted to account for whether a single dose is being administered or whether multiple doses are being administered. The precise determination of what would be considered an effective dose can be based on factors individual to each subject.

As used herein, the terms “treat,” “treating,” and “treatment” are all intended to refer to an amelioration or reversal of at least one measurable physical parameter related to a cancer and/or an inflammatory disease, disorder or condition, which is not necessarily discernible in the subject, but can be discernible in the subject. The terms “treat,” “treating,” and “treatment,” can also refer to causing regression, preventing the progression, or at least slowing down the progression of the disease, disorder, or condition. In a particular embodiment, “treat,” “treating,” and “treatment” refer to an alleviation, prevention of the development or onset, or reduction in the duration of one or more symptoms associated with the disease, disorder, or condition, such as a tumor or more preferably a cancer. In a particular embodiment, “treat,” “treating,” and “treatment” refer to prevention of the recurrence of the disease, disorder, or condition. In a particular embodiment, “treat,” “treating,” and “treatment” refer to an increase in the survival of a subject having the disease, disorder, or condition. In a particular embodiment, “treat,” “treating,” and “treatment” refer to elimination of the disease, disorder, or condition in the subject.

According to particular embodiments, provided are compositions used in the treatment of a cancer and/or an inflammatory disease, disorder or condition. For cancer therapy, the provided compositions can be used in combination with another treatment including, but not limited to, a chemotherapy, an anti-CD20 mAb, an anti-TIM-3 mAb, an anti-LAG-3 mAb, an anti-EGFR mAb, an anti-HER-2 mAb, an anti-CD19 mAb, an anti-CD33 mAb, an anti-CD47 mAb, an anti-CD73 mAb, an anti-Claudin18.2 mAb, an anti-apelin mAb, an anti-TIP-1 mAb, an anti-FOLR1 mAb, an anti-CTLA-4 mAb, an anti-PD-L1 mAb, an anti-PD-1 mAb, other immuno-oncology drugs, an antiangiogenic agent, a radiation therapy, an antibody-drug conjugate (ADC), a targeted therapy, or other anticancer drugs.

According to particular embodiments, the methods of treating cancer in a subject in need thereof comprise administering to the subject the CAR-T cells and/or CAR-NK cells of the invention in combination with an agent that increases the efficacy of a cell expressing a CAR molecule. Such agents include, but not limited to, antibody fragment that binds to CD73, CD39, PD1, PD-L1, PD-L2, CTLA4, TIM3 or LAG3, or an adenosine A2a receptor antagonist.

According to particular embodiments, the methods of treating cancer in a subject in need thereof comprise administering to the subject the CAR-T cells and/or CAR-NK cells of the invention in combination with an agent that ameliorates one or more side effects associated with administration of a cell expressing a CAR molecule. Such agents include, but not limited to, a steroid, an inhibitor of TNFα, or an inhibitor of IL-6.

According to particular embodiments, the methods of treating cancer in a subject in need thereof comprise administering to the subject the CAR-T cells and/or CAR-NK cells of the invention in combination with an agent that treats the disease associated with DLL3. Such agents include, but not limited to, an anti-DLL3 monoclonal antibody or bispecific antibody.

As used herein, the term “in combination,” in the context of the administration of two or more therapies to a subject, refers to the use of more than one therapy. The use of the term “in combination” does not restrict the order in which therapies are administered to a subject. For example, a first therapy (e.g., a composition described herein) can be administered prior to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject.

### EMBODIMENTS

The invention provides also the following non-limiting embodiments.

Embodiment 1 is an isolated polynucleotide comprising a nucleic acid sequence encoding a chimeric antigen receptor (CAR), wherein the CAR comprises: (a) an extracellular domain comprising at least one antigen binding domain that specifically binds DLL3; (b) a hinge region; (c) a transmembrane region; and (d) an intracellular signaling domain.

Embodiment 2 is the isolated polynucleotide of embodiment 1, wherein the antigen binding domain comprises a heavy chain complementarity determining region 1 (HCDR1), HCDR2, HCDR3, a light chain complementarity determining region 1 (LCDR1), LCDR2, and LCDR3, having the polypeptide sequences of:
(1) SEQ ID NOs: 25, 26, 27, 61, 62 and 63, respectively;
(2) SEQ ID NOs: 28, 29, 30, 64, 65 and 66, respectively;
(3) SEQ ID NOs: 31, 32, 33, 67, 68 and 69, respectively;
(4) SEQ ID NOs: 34, 35, 36, 70, 71 and 72, respectively;
(5) SEQ ID NOs: 37, 38, 39, 73, 74 and 75, respectively;
(6) SEQ ID NOs: 40, 41, 42, 76, 77 and 78, respectively;
(7) SEQ ID NOs: 43, 44, 45, 79, 80 and 81, respectively;
(8) SEQ ID NOs: 46, 47, 48, 82, 83 and 84, respectively;
(9) SEQ ID NOs: 49, 50, 51, 85, 86 and 87, respectively;
(10) SEQ ID NOs: 52, 53, 54, 88, 89 and 90, respectively;
(11) SEQ ID NOs: 55, 56, 57, 91, 92 and 93, respectively; or
(12) SEQ ID NOs: 58, 59, 60, 94, 95 and 96, respectively;
   wherein the antigen binding domain specifically binds DLL3, preferably human DLL3.

Embodiment 3 is the isolated polynucleotide of embodiment 1, wherein the antigen binding domain comprises a heavy chain complementarity determining region 1 (HCDR1), HCDR2, HCDR3, a light chain complementarity determining region 1 (LCDR1), LCDR2, and LCDR3, having the polypeptide sequences of:
(1) SEQ ID NOs: 97, 98, 99, 133, 134 and 135, respectively;
(2) SEQ ID NOs: 100, 101, 102, 136, 137 and 138, respectively;
(3) SEQ ID NOs: 103, 104, 105, 139, 140 and 141, respectively;
(4) SEQ ID NOs: 106, 107, 108, 142, 143 and 144, respectively;
(5) SEQ ID NOs: 109, 110, 111, 145, 146 and 147, respectively;
(6) SEQ ID NOs: 112, 113, 114, 148, 149 and 150, respectively;
(7) SEQ ID NOs: 115, 116, 117, 151, 152 and 153, respectively;
(8) SEQ ID NOs: 118, 119, 120, 154, 155 and 156, respectively;
(9) SEQ ID NOs: 121, 122, 123, 157, 158 and 159, respectively;
(10) SEQ ID NOs: 124, 125, 126, 160, 161 and 162, respectively;
(11) SEQ ID NOs: 127, 128, 129, 163, 164 and 165, respectively; or
(12) SEQ ID NOs: 130, 131, 132, 166, 167 and 168, respectively;
   wherein the antigen binding domain specifically binds DLL3, preferably human DLL3.

Embodiment 4 is the isolated polynucleotide of any one of embodiments 1-3, wherein the antigen binding domain comprises a heavy chain variable region having a polypeptide sequence at least 95%, at least 96%, at least 97%, at least 98%, or at least 99% identical to SEQ ID NO: 1, 3, 5, 7, 9, 11, 13, 15, 17, 19, 21, or 23, or a light chain variable region having a polypeptide sequence at least 95%, at least 96%, at least 97%, at least 98%, or at least 99% identical to SEQ ID NO: 2, 4, 6, 8, 10, 12, 14, 16, 18, 20, 22, or 24.

Embodiment 5 is the isolated polynucleotide of any one of embodiments 1-4, wherein the antigen binding domain comprises:
(1) a heavy chain variable region having the polypeptide sequence of SEQ ID NO:1, and a light chain variable region having the polypeptide sequence of SEQ ID NO:2;
(2) a heavy chain variable region having the polypeptide sequence of SEQ ID NO:3, and a light chain variable region having the polypeptide sequence of SEQ ID NO:4;
(3) a heavy chain variable region having the polypeptide sequence of SEQ ID NO:5, and a light chain variable region having the polypeptide sequence of SEQ ID NO:6;
(4) a heavy chain variable region having the polypeptide sequence of SEQ ID NO:7, and a light chain variable region having the polypeptide sequence of SEQ ID NO:8;
(5) a heavy chain variable region having the polypeptide sequence of SEQ ID NO:9, and a light chain variable region having the polypeptide sequence of SEQ ID NO:10;
(6) a heavy chain variable region having the polypeptide sequence of SEQ ID NO:11, and a light chain variable region having the polypeptide sequence of SEQ ID NO:12;
(7) a heavy chain variable region having the polypeptide sequence of SEQ ID NO:13, and a light chain variable region having the polypeptide sequence of SEQ ID NO:14;
(8) a heavy chain variable region having the polypeptide sequence of SEQ ID NO:15, and a light chain variable region having the polypeptide sequence of SEQ ID NO:16;
(9) a heavy chain variable region having the polypeptide sequence of SEQ ID NO:17, and a light chain variable region having the polypeptide sequence of SEQ ID NO:18;
(10) a heavy chain variable region having the polypeptide sequence of SEQ ID NO:19, and a light chain variable region having the polypeptide sequence of SEQ ID NO:20;
(11) a heavy chain variable region having the polypeptide sequence of SEQ ID NO:21, and a light chain variable region having the polypeptide sequence of SEQ ID NO:22; or
(12) a heavy chain variable region having the polypeptide sequence of SEQ ID NO:23, and a light chain variable region having the polypeptide sequence of SEQ ID NO:24.

Embodiment 6 is the isolated polynucleotide of any one of embodiments 1-4, wherein the antigen binding domain is humanized and comprises a heavy chain variable region having a polypeptide sequence at least 95%, at least 96%, at least 97%, at least 98%, or at least 99% identical to any one of SEQ ID NOs: 170, 175-209 or 248-255, or a light chain variable region having a polypeptide sequence at least 95%, at least 96%, at least 97%, at least 98%, or at least 99% identical to any one of SEQ ID NOs: 171-174, 210-240 or 256-264.

Embodiment 7 is the isolated polynucleotide of embodiment 6, wherein the antigen binding domain is humanized and comprises:
(1) a heavy chain variable region having the polypeptide sequence of SEQ ID NO:170, and a light chain variable region having the polypeptide sequence of SEQ ID NO:171;
(2) a heavy chain variable region having the polypeptide sequence of SEQ ID NO:170, and a light chain variable region having the polypeptide sequence of SEQ ID NO:172;
(3) a heavy chain variable region having the polypeptide sequence of SEQ ID NO:170, and a light chain variable region having the polypeptide sequence of SEQ ID NO:173;
(4) a heavy chain variable region having the polypeptide sequence of SEQ ID NO:183, and a light chain variable region having the polypeptide sequence of SEQ ID NO:217;
(5) a heavy chain variable region having the polypeptide sequence of SEQ ID NO:183, and a light chain variable region having the polypeptide sequence of SEQ ID NO:218;
(6) a heavy chain variable region having the polypeptide sequence of SEQ ID NO:184, and a light chain variable region having the polypeptide sequence of SEQ ID NO:217;
(7) a heavy chain variable region having the polypeptide sequence of SEQ ID NO:184, and a light chain variable region having the polypeptide sequence of SEQ ID NO:218;
(8) a heavy chain variable region having the polypeptide sequence of SEQ ID NO:198, and a light chain variable region having the polypeptide sequence of SEQ ID NO:229;
(9) a heavy chain variable region having the polypeptide sequence of SEQ ID NO:200, and a light chain variable region having the polypeptide sequence of SEQ ID NO:229;
(10) a heavy chain variable region having the polypeptide sequence of SEQ ID NO:198, and a light chain variable region having the polypeptide sequence of SEQ ID NO:231;
(11) a heavy chain variable region having the polypeptide sequence of SEQ ID NO:200, and a light chain variable region having the polypeptide sequence of SEQ ID NO:231;
(12) a heavy chain variable region having the polypeptide sequence of SEQ ID NO:201, and a light chain variable region having the polypeptide sequence of SEQ ID NO:229;
(13) a heavy chain variable region having the polypeptide sequence of SEQ ID NO:201, and a light chain variable region having the polypeptide sequence of SEQ ID NO:230;
(14) a heavy chain variable region having the polypeptide sequence of SEQ ID NO:201, and a light chain variable region having the polypeptide sequence of SEQ ID NO:231;
(15) a heavy chain variable region having the polypeptide sequence of SEQ ID NO:175, and a light chain variable region having the polypeptide sequence of SEQ ID NO:210;
(16) a heavy chain variable region having the polypeptide sequence of SEQ ID NO:175, and a light chain variable region having the polypeptide sequence of SEQ ID NO:211;
(17) a heavy chain variable region having the polypeptide sequence of SEQ ID NO:175, and a light chain variable region having the polypeptide sequence of SEQ ID NO:212;
(18) a heavy chain variable region having the polypeptide sequence of SEQ ID NO:176, and a light chain variable region having the polypeptide sequence of SEQ ID NO:210;
(19) a heavy chain variable region having the polypeptide sequence of SEQ ID NO:176, and a light chain variable region having the polypeptide sequence of SEQ ID NO:211;
(20) a heavy chain variable region having the polypeptide sequence of SEQ ID NO:176, and a light chain variable region having the polypeptide sequence of SEQ ID NO:212;
(21) a heavy chain variable region having the polypeptide sequence of SEQ ID NO:177, and a light chain variable region having the polypeptide sequence of SEQ ID NO:210;
(22) a heavy chain variable region having the polypeptide sequence of SEQ ID NO:177, and a light chain variable region having the polypeptide sequence of SEQ ID NO:211;
(23) a heavy chain variable region having the polypeptide sequence of SEQ ID NO:178, and a light chain variable region having the polypeptide sequence of SEQ ID NO:210;
(24) a heavy chain variable region having the polypeptide sequence of SEQ ID NO:178, and a light chain variable region having the polypeptide sequence of SEQ ID NO:211;
(25) a heavy chain variable region having the polypeptide sequence of SEQ ID NO:177, and a light chain variable region having the polypeptide sequence of SEQ ID NO:211;
(26) a heavy chain variable region having the polypeptide sequence of SEQ ID NO:177, and a light chain variable region having the polypeptide sequence of SEQ ID NO:212;
(27) a heavy chain variable region having the polypeptide sequence of SEQ ID NO:178, and a light chain variable region having the polypeptide sequence of SEQ ID NO:212;
(28) a heavy chain variable region having the polypeptide sequence of SEQ ID NO:179, and a light chain variable region having the polypeptide sequence of SEQ ID NO:213;
(29) a heavy chain variable region having the polypeptide sequence of SEQ ID NO:179, and a light chain variable region having the polypeptide sequence of SEQ ID NO:214;
(30) a heavy chain variable region having the polypeptide sequence of SEQ ID NO:179, and a light chain variable region having the polypeptide sequence of SEQ ID NO:215;
(31) a heavy chain variable region having the polypeptide sequence of SEQ ID NO:180, and a light chain variable region having the polypeptide sequence of SEQ ID NO:213;
(32) a heavy chain variable region having the polypeptide sequence of SEQ ID NO:180, and a light chain variable region having the polypeptide sequence of SEQ ID NO:214;
(33) a heavy chain variable region having the polypeptide sequence of SEQ ID NO:180, and a light chain variable region having the polypeptide sequence of SEQ ID NO:215;
(34) a heavy chain variable region having the polypeptide sequence of SEQ ID NO:181, and a light chain variable region having the polypeptide sequence of SEQ ID NO:213;
(35) a heavy chain variable region having the polypeptide sequence of SEQ ID NO:181, and a light chain variable region having the polypeptide sequence of SEQ ID NO:214;
(36) a heavy chain variable region having the polypeptide sequence of SEQ ID NO:182, and a light chain variable region having the polypeptide sequence of SEQ ID NO:215;
(37) a heavy chain variable region having the polypeptide sequence of SEQ ID NO:202, and a light chain variable region having the polypeptide sequence of SEQ ID NO:232;
(38) a heavy chain variable region having the polypeptide sequence of SEQ ID NO:202, and a light chain variable region having the polypeptide sequence of SEQ ID NO:233;
(39) a heavy chain variable region having the polypeptide sequence of SEQ ID NO:202, and a light chain variable region having the polypeptide sequence of SEQ ID NO:234;
(40) a heavy chain variable region having the polypeptide sequence of SEQ ID NO:203, and a light chain variable region having the polypeptide sequence of SEQ ID NO:232;
(41) a heavy chain variable region having the polypeptide sequence of SEQ ID NO:203, and a light chain variable region having the polypeptide sequence of SEQ ID NO:233;
(42) a heavy chain variable region having the polypeptide sequence of SEQ ID NO:203, and a light chain variable region having the polypeptide sequence of SEQ ID NO:234;
(43) a heavy chain variable region having the polypeptide sequence of SEQ ID NO:204, and a light chain variable region having the polypeptide sequence of SEQ ID NO:234;
(44) a heavy chain variable region having the polypeptide sequence of SEQ ID NO:208, and a light chain variable region having the polypeptide sequence of SEQ ID NO:239;
(45) a heavy chain variable region having the polypeptide sequence of SEQ ID NO:208, and a light chain variable region having the polypeptide sequence of SEQ ID NO:240;
(46) a heavy chain variable region having the polypeptide sequence of SEQ ID NO:253, and a light chain variable region having the polypeptide sequence of SEQ ID NO:261; or
(47) a heavy chain variable region having the polypeptide sequence of SEQ ID NO:255, and a light chain variable region having the polypeptide sequence of SEQ ID NO:263.

Embodiment 8 is the isolated polynucleotide of any one of embodiments 1-7, wherein the antigen binding domain is a single chain variable fragment (scFv) that specifically binds DLL3, preferably human DLL3.

Embodiment 9 is the isolated polynucleotide of embodiment 8, wherein the single chain variable fragment (scFv) is humanized.

Embodiment 10 is the isolated polynucleotide of embodiment 8 or 9, wherein the single chain variable fragment (scFv) comprises a polypeptide sequence at least 95% identical to any one of SEQ ID NOs:241-247 or 265-286.

Embodiment 11 is the isolated polynucleotide of any one of embodiments 1-10, wherein the chimeric antigen receptor (CAR) comprises one or more antigen binding domains.

Embodiment 12 is the isolated polynucleotide of any one of embodiments 1-11, wherein the intracellular signaling domain of the CAR comprises one or more costimulatory domains and one or more activating domains.

Embodiment 13 is a chimeric antigen receptor (CAR) encoded by the isolated polynucleotide of any one of embodiments 1-12.

Embodiment 14 is a vector comprising the isolated polynucleotide of any one of embodiments 1-12.

Embodiment 15 is a host cell comprising the vector of embodiment 14.

Embodiment 16 is the host cell of embodiment 15, wherein the cell is a CAR-T cell, preferably a human CAR-T cell.

Embodiment 17 is the host cell of embodiment 15, wherein the cell is a CAR-NK cell, preferably a human CAR-NK cell.

Embodiment 18 is a method of making a host cell expressing a chimeric antigen receptor (CAR), the method comprising transducing a T cell with the vector of embodiment 14.

Embodiment 19 is a method of producing a chimeric antigen receptor (CAR)-T cell, the method comprising culturing T cells comprising the isolated polynucleotide comprising a nucleic acid encoding a chimeric antigen receptor (CAR) of any one of embodiments 1-12 under conditions to produce the CAR-T cell and recovering the CAR-T cell.

Embodiment 20 is a method of making a host cell expressing a chimeric antigen receptor (CAR), the method comprising transducing a NK cell with the vector of embodiment 14.

Embodiment 21 is a method of producing a chimeric antigen receptor (CAR)-NK cell, the method comprising culturing NK cells comprising the isolated polynucleotide comprising a nucleic acid encoding a chimeric antigen receptor (CAR) of any one of embodiments 1-12 under conditions to produce the CAR-NK cell, and recovering the CAR-NK cell.

Embodiment 22 is a method of generating the cell comprising a chimeric antigen receptor (CAR), the method comprising contacting a cell with the isolated polynucleotide comprising a nucleic acid encoding a chimeric antigen receptor (CAR) of any one of embodiments 1-12, wherein the isolated polynucleotide is an in vitro transcribed RNA or synthetic RNA.

Embodiment 23 is a method of treating cancer in a subject in need thereof, the method comprising administering to the subject the host cell of any one of embodiments 15-17.

Embodiment 24 is the method of embodiment 23, wherein the cancer is selected from a lung cancer such as small cell lung cancer (SCLC), large cell neuroendocrine carcinoma (LCNEC), a gastric cancer, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, and other solid tumors, and a non-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

Embodiment 25 is the method of embodiment 23 or 24, further comprising administering to the subject in need thereof an agent that increases the efficacy of a cell expressing a CAR molecule.

Embodiment 26 is the method of embodiment 23 or 24, further comprising administering to the subject in need thereof an agent that ameliorates one or more side effects associated with administration of a cell expressing a CAR molecule.

Embodiment 27 is the method of embodiment 23 or 24, further comprising administering to the subject in need thereof an agent that treats the disease associated with DLL3.

Embodiment 28 is a humanized anti-DLL3 monoclonal antibody or antigen-binding fragment thereof, wherein the antibody or antigen-binding fragment thereof comprises a heavy chain variable region having a polypeptide sequence at least 95% identical to any one of SEQ ID NOs: 170, 175-209 or 248-255, or a light chain variable region having a polypeptide sequence at least 95% identical to any one of SEQ ID NOs: 171-174, 210-240 or 256-264.

Embodiment 29 is the humanized anti-DLL3 monoclonal antibody or antigen-binding fragment thereof of embodiment 28, wherein the antibody or antigen-binding fragment thereof comprises:
(1) a heavy chain variable region having the polypeptide sequence of SEQ ID NO:170, and a light chain variable region having the polypeptide sequence of SEQ ID NO:171;
(2) a heavy chain variable region having the polypeptide sequence of SEQ ID NO:170, and a light chain variable region having the polypeptide sequence of SEQ ID NO:172;
(3) a heavy chain variable region having the polypeptide sequence of SEQ ID NO:170, and a light chain variable region having the polypeptide sequence of SEQ ID NO:173.
(4) a heavy chain variable region having the polypeptide sequence of SEQ ID NO:183, and a light chain variable region having the polypeptide sequence of SEQ ID NO:217;
(5) a heavy chain variable region having the polypeptide sequence of SEQ ID NO:183, and a light chain variable region having the polypeptide sequence of SEQ ID NO:218;
(6) a heavy chain variable region having the polypeptide sequence of SEQ ID NO:184, and a light chain variable region having the polypeptide sequence of SEQ ID NO:217;
(7) a heavy chain variable region having the polypeptide sequence of SEQ ID NO:184, and a light chain variable region having the polypeptide sequence of SEQ ID NO:218;
(8) a heavy chain variable region having the polypeptide sequence of SEQ ID NO:198, and a light chain variable region having the polypeptide sequence of SEQ ID NO:229;
(9) a heavy chain variable region having the polypeptide sequence of SEQ ID NO:200, and a light chain variable region having the polypeptide sequence of SEQ ID NO:229;
(10) a heavy chain variable region having the polypeptide sequence of SEQ ID NO:198, and a light chain variable region having the polypeptide sequence of SEQ ID NO:231;
(11) a heavy chain variable region having the polypeptide sequence of SEQ ID NO:200, and a light chain variable region having the polypeptide sequence of SEQ ID NO:231;
(12) a heavy chain variable region having the polypeptide sequence of SEQ ID NO:201, and a light chain variable region having the polypeptide sequence of SEQ ID NO:229;
(13) a heavy chain variable region having the polypeptide sequence of SEQ ID NO:201, and a light chain variable region having the polypeptide sequence of SEQ ID NO:230;
(14) a heavy chain variable region having the polypeptide sequence of SEQ ID NO:201, and a light chain variable region having the polypeptide sequence of SEQ ID NO:231;
(15) a heavy chain variable region having the polypeptide sequence of SEQ ID NO:175, and a light chain variable region having the polypeptide sequence of SEQ ID NO:210;
(16) a heavy chain variable region having the polypeptide sequence of SEQ ID NO:175, and a light chain variable region having the polypeptide sequence of SEQ ID NO:211;
(17) a heavy chain variable region having the polypeptide sequence of SEQ ID NO:175, and a light chain variable region having the polypeptide sequence of SEQ ID NO:212;
(18) a heavy chain variable region having the polypeptide sequence of SEQ ID NO:176, and a light chain variable region having the polypeptide sequence of SEQ ID NO:210;
(19) a heavy chain variable region having the polypeptide sequence of SEQ ID NO:176, and a light chain variable region having the polypeptide sequence of SEQ ID NO:211;
(20) a heavy chain variable region having the polypeptide sequence of SEQ ID NO:176, and a light chain variable region having the polypeptide sequence of SEQ ID NO:212;
(21) a heavy chain variable region having the polypeptide sequence of SEQ ID NO:177, and a light chain variable region having the polypeptide sequence of SEQ ID NO:210;
(22) a heavy chain variable region having the polypeptide sequence of SEQ ID NO:177, and a light chain variable region having the polypeptide sequence of SEQ ID NO:211;
(23) a heavy chain variable region having the polypeptide sequence of SEQ ID NO:178, and a light chain variable region having the polypeptide sequence of SEQ ID NO:210;
(24) a heavy chain variable region having the polypeptide sequence of SEQ ID NO:178, and a light chain variable region having the polypeptide sequence of SEQ ID NO:211;
(25) a heavy chain variable region having the polypeptide sequence of SEQ ID NO:177, and a light chain variable region having the polypeptide sequence of SEQ ID NO:211;
(26) a heavy chain variable region having the polypeptide sequence of SEQ ID NO:177, and a light chain variable region having the polypeptide sequence of SEQ ID NO:212;
(27) a heavy chain variable region having the polypeptide sequence of SEQ ID NO:178, and a light chain variable region having the polypeptide sequence of SEQ ID NO:212;
(28) a heavy chain variable region having the polypeptide sequence of SEQ ID NO:179, and a light chain variable region having the polypeptide sequence of SEQ ID NO:213;
(29) a heavy chain variable region having the polypeptide sequence of SEQ ID NO:179, and a light chain variable region having the polypeptide sequence of SEQ ID NO:214;
(30) a heavy chain variable region having the polypeptide sequence of SEQ ID NO:179, and a light chain variable region having the polypeptide sequence of SEQ ID NO:215;
(31) a heavy chain variable region having the polypeptide sequence of SEQ ID NO:180, and a light chain variable region having the polypeptide sequence of SEQ ID NO:213;
(32) a heavy chain variable region having the polypeptide sequence of SEQ ID NO:180, and a light chain variable region having the polypeptide sequence of SEQ ID NO:214;
(33) a heavy chain variable region having the polypeptide sequence of SEQ ID NO:180, and a light chain variable region having the polypeptide sequence of SEQ ID NO:215;
(34) a heavy chain variable region having the polypeptide sequence of SEQ ID NO:181, and a light chain variable region having the polypeptide sequence of SEQ ID NO:213;
(35) a heavy chain variable region having the polypeptide sequence of SEQ ID NO:181, and a light chain variable region having the polypeptide sequence of SEQ ID NO:214;
(36) a heavy chain variable region having the polypeptide sequence of SEQ ID NO:182, and a light chain variable region having the polypeptide sequence of SEQ ID NO:215;
(37) a heavy chain variable region having the polypeptide sequence of SEQ ID NO:202, and a light chain variable region having the polypeptide sequence of SEQ ID NO:232;
(38) a heavy chain variable region having the polypeptide sequence of SEQ ID NO:202, and a light chain variable region having the polypeptide sequence of SEQ ID NO:233;
(39) a heavy chain variable region having the polypeptide sequence of SEQ ID NO:202, and a light chain variable region having the polypeptide sequence of SEQ ID NO:234;
(40) a heavy chain variable region having the polypeptide sequence of SEQ ID NO:203, and a light chain variable region having the polypeptide sequence of SEQ ID NO:232;
(41) a heavy chain variable region having the polypeptide sequence of SEQ ID NO:203, and a light chain variable region having the polypeptide sequence of SEQ ID NO:233;
(42) a heavy chain variable region having the polypeptide sequence of SEQ ID NO:203, and a light chain variable region having the polypeptide sequence of SEQ ID NO:234;
(43) a heavy chain variable region having the polypeptide sequence of SEQ ID NO:204, and a light chain variable region having the polypeptide sequence of SEQ ID NO:234;
(44) a heavy chain variable region having the polypeptide sequence of SEQ ID NO:208, and a light chain variable region having the polypeptide sequence of SEQ ID NO:239;
(45) a heavy chain variable region having the polypeptide sequence of SEQ ID NO:208, and a light chain variable region having the polypeptide sequence of SEQ ID NO:240;
(46) a heavy chain variable region having the polypeptide sequence of SEQ ID NO:253, and a light chain variable region having the polypeptide sequence of SEQ ID NO:261; or
(47) a heavy chain variable region having the polypeptide sequence of SEQ ID NO:255, and a light chain variable region having the polypeptide sequence of SEQ ID NO:263.

Embodiment 30 is the humanized anti-DLL3 monoclonal antibody or antigen-binding fragment thereof of any one of embodiment 28 or 29, wherein the monoclonal antibody or antigen-binding fragment thereof is capable of inducing effector-mediated tumor cell lysis, mediating the recruitment of conjugated drugs, and/or forms a bispecific antibody with another monoclonal antibody or antigen-binding fragment thereof with a cancer-killing effect.

Embodiment 31 is an isolated nucleic acid encoding the monoclonal antibody or antigen-binding fragment thereof of any one of embodiments 28-30.

Embodiment 32 is a vector comprising the isolated nucleic acid of embodiment 31.

Embodiment 33 is a host cell comprising the vector of embodiment 32.

Embodiment 34 is a pharmaceutical composition, comprising the isolated monoclonal antibody or antigen-binding fragment thereof of any one of embodiments 28-30 and a pharmaceutically acceptable carrier.

Embodiment 35 is a method of targeting DLL3 on a cancer cell surface in a subject in need thereof, comprising administering to the subject in need thereof a pharmaceutical composition comprising the humanized anti-DLL3 monoclonal antibody or antigen-binding fragment thereof of any one of embodiments 28-30.

Embodiment 36 is a method of treating cancer in a subject in need thereof, comprising administering to the subject the pharmaceutical composition of embodiment 34.

Embodiment 37 is the method of embodiment 36, wherein the cancer is selected from a lung cancer such as small cell lung cancer (SCLC), large cell neuroendocrine carcinoma (LCNEC), a gastric cancer, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, and other solid tumors, and a non-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

Embodiment 38 is a method of producing the monoclonal antibody or antigen-binding fragment thereof of any one of embodiments 28-30, comprising culturing a cell comprising a nucleic acid encoding the monoclonal antibody or antigen-binding fragment thereof under conditions to produce the monoclonal antibody or antigen-binding fragment thereof, and recovering the antibody or antigen-binding fragment thereof from the cell or culture.

Embodiment 39 is a method of producing a pharmaceutical composition comprising the monoclonal antibody or antigen-binding fragment of any one of embodiments 28-30, comprising combining the monoclonal antibody or antigen-binding fragment thereof with a pharmaceutically acceptable carrier to obtain the pharmaceutical composition.

### EXAMPLES

### Example 1: Identification of Antigen Binding Domains that Specifically Bind DLL3

The antigen binding domains that specifically bind DLL3 are anti-DLL3 mAbs isolated and sequenced as described in PCT Patent Application No. PCT/US2019/029888, filed on Apr. 30, 2019, which is incorporated herein by reference in its entirety.

Sequences of heavy and light chain variable regions for the antigen binding domains that specifically bind DLL3 are provided in Tables 1 and 2, and the CDR regions for the antigen binding domains that specifically bind DLL3 are provided in Tables 3-6.

TABLE 1
Sequences of heavy chain variable regions for the antigen
binding domains that specifically bind DLL3

| | | SEQ |
|---|---|---|
| | | ID |
| Name | VH | NO: |
| 13P9A | EVQLQQSGPELVKPGASVKMSCKASGYTFTSYVMHWVKQKPGQGPDWIG | 1 |
| | YINPYNDATKYNEKFKGKATLTSDKSSSTAYMELSSLTSEDSAVYYCARGG | |
| | YDYDGDYWGQGTTLTVSS | |
| 5A16A | EVQLQQSGPELVKPGASVKMSCKASGYTFTRYILHWVKLKPGQGLEWIGYI | 3 |
| | NPYNDGTKYNEKFKGKATLTSDKSSSTAYMELSRLTSYDSAVYYCARDSSG | |
| | YGGAYAMDFWGQGTSVTVSS | |
| 14L22A | EVQLVESGGGLVKPGGSLKLSCAASGFTFSSYAMSWVRQTPEKRLEWVAAI | 5 |
| | NSNGGNTYYPDTVKDRFTISRDNAKNTLYLQMSSLRSEDTALYYCARHRGG | |
| | FYYAVDYWGQGTSVTVSS | |
| 10P18A | EVQLQQSGPELVKPGASVKISCKASGYSFTGYYIDWVKQSPGKSLEWIGYIY | 7 |
| | PSNGETSYNQKFKGKATLTVDKSSSTVNMQLNSLTSEDSAVYYCARESYAM | |
| | DYWGQGTSVTVSS | |
| 13P11A | DVQLQESGPGLVKPSQTVSLTCTVTGYSITNGNHWWSWIRQVSGSKLEWM | 9 |
| | GYISSSGSTDSNPSLKSRISITRDTSKNQLFLHLNSVTTEDIATYYCATTGT | |
| | WGYFDYWGQGTTLTVSS | |
| 3C16A | EVQLQQSGPELVKPGTSVKMSCKASGYTFTSYVMHWVKQKPGQGLEWIGY | 11 |
| | VIPYNDGTKYNEKFKGKATLTSDKSSSTAYMELSSLTSEDSAVYYCARPSN | |
| | WDEFDYWGQGTTLTVSS | |
| 3I21A | QVQLQQPGAELVKPGASVKLSCKASGYTFTNYWMNWVKQRPGRGLEWIG | 13 |
| | RIHPSDSETHYNQKFKTKATLTVDKSSSTAYIQLSSLTSEDSAVYYCARYDG | |
| | YFAYWGQGTLVTVSA | |
| 8H5A | QVTLKESGPGILQPSQTLSLTCSFSGFSLSTFGMGVGWIRQPSGKGLEWLAHI | 15 |
| | WWDDDKYYNPALKSRLTISKDTSKNQVFLKIANVDIADTATYYCARTYDY | |
| | DEYFDYWGQGTTLTVSS | |
| 15K2A | QVQLQQPGAELVQPGASVKLSCKASGYTFTSYWMNWMKQRPGRGLEWIG | 17 |
| | RIHPSDSETHYNQKFRTKATLTVDKSSSTAYIQLSSLTSEDSAVYYCAREDG | |
| | YYWYFDVWGAGTTVTVSS | |
| 5A24A | EVQLQQSGAELVKPGASVKIPCKASGYKFTDFNMDWVKQSHGKSLEWIGDI | 19 |
| | NPNSGGTIYNQKFKGKATLTVDKSLSTAYMELGSLTSEDTAVYYCARWDY | |
| | GNFAYWGQGTLVTVSA | |
| 15P17A | QVQLQQPGAELVKPGASVKLSCKASGYTFTNYWMNWVKQRPGRGLEWIG | 21 |
| | RIHPSDSETHYNQKFKSKATLTVDKSSSTAYIQLSSLTSEDSAVYYCAREDG | |
| | YYWYFDVWGAGTTVTVSS | |
| 15N21A | EVQLVESGGGLVKPGGSLKLSCAASGFTFSSYAMSWVRQTPEKRLEWVAAI | 23 |
| | NSNGGRNYYPDTVKDRFTISRDNAKNTLYLQMSSLRSEDTALYYCARHRG | |
| | GYYYAMDYWGQGTSVTVSS | |

VH: heavy chain variable region

TABLE 2
Sequences of light chain variable regions for the antigen
binding domains that specifically bind DLL3

| | | SEQ |
|---|---|---|
| | | ID |
| Name | VL | NO: |
| 13P9A | DIQMNQSPSSLSASLGDSITITCHASQNINVWLSWYQQKPGNIPKLLIYKASNLHTGVP | 2 |
| | SRFSGSGSGTGFTLTISSLQPEDIATYYCQQGQSYPFTFGSGTKLEIK | |
| 5A16A | DIQMTQSPASLSASVGETVTITCRASGNIHNYLAWYQQKQGRSPQLLVYNAKTLPYG | 4 |
| | VPSRFSGSGSGTQYSLKINSLQPEDFGSYYCQHFWTTPWTFGGGTKLEIK | |
| 14L22A | NIMMTQSPSSLAVSAGEKVTMSCKSSQSVLYSSNQKNYLAWYQQKPGQSPKLLIYWA | 6 |
| | STRESGVPDRFTGSGSGTDFTLTISSVQAEDLAVYYCHQYLSSRTFGGGTKLEIK | |
| 10P18A | DIVLTQSPASLAVSLGQRATISCRASKSVSTSGYSYMHWYQQKPGQPPKLLIYLASNLE | 8 |
| | SGVPARFSGSGSGTDFTLNIHPVEEEDAATYYCQHSRELPYTFGGGTKLEIK | |
| 13P11A | NIVMTQSPKSMSMSVGERVTLSCKASENVGTYVSWYQQKPEQSPKLLIYGASNRFTG | 10 |
| | VPDRFTGSGSATDFTLTISSVQAEDLADYHCGQSYSYPFTFGSGTKLEIK | |
| 3C16A | DIVMTQSQKFMSTSVGDRVSITCKASQNVRTAVAWYQQKPGQSPKALIYLASNRHTG | 12 |
| | VPDRFTGSGSGTDFTLTISNVQSEDLADYFCLQHWNYPLTFGAGTKLELK | |
| 3I21A | DIQMTQSSSYLSVSLGGRVTITCKASDHINNWLAWYQQKPGNAPRLLISGATSLETGD | 14 |
| | PSRFSGSGSGKDYTLSITSLQIEDVATYYCQQYWSIPFTFGAGTKLELK | |
| 8H5A | DIVMTQAAFSNPVTLGTSASISCRSSKSLLHSNGITYFYWYLQKPGQSPQLLIYQMSNL | 16 |
| | ASGVPDRFSSSGSGTDFTLRISRVEAEDVGVYYCAQNLELPFTFGSGTKLEIK | |
| 15K2A | NIVLTQSPASLAVSLGQRATISCRASESVDIYGNSFMHWYQQKPGQPPKLLIYLASNLE | 18 |
| | SGVPARFSGSGSRTDFTLTIDPVEADDAATYYCQQNNEDPWTFGGGTKLEIK | |
| 5A24A | DIVMTQAAFSNPVTLGTSASISCRSSKSLLHSNGITYLYWYLQKPGQSPQLLIYQMSNL | 20 |
| | ASGVPDRFSSSGSGTDFTLRISRVEAEDVGVYYCAQNLELPLTFGAGTKLELK | |
| 15P17A | NIVLTQSPASLAVSLGQRATISCRASESVDSYGNSFMHWYQQKPGQPPKLLIYLASNLE | 22 |
| | SGVPARFSGSGSRTDFTLTIDPVEADDAATYYCQQNHEDPWTFGGGTKLEIK | |
| 15N21A | DIVMSQSPSSLAVSVGEKVTMSCKSSQSLLYSSNQKNYLAWYQQKPGQSPKLLIYWA | 24 |
| | STRESGVPDRFTGSGSGTDFTLTISSVKAEDLAVYYCQQYYTYLTFGAGTKLELK | |

VL: light chain variable region

TABLE 3
CDR regions 1-3 of heavy chain for the antigen binding
domains that specifically bind DLL3

| Name | HC CDR1 | NO | HC CDR2 | NO | HC CDR3 | NO |
|---|---|---|---|---|---|---|
| 13P9A | GYTFTSYV | 25 | INPYNDAT | 26 | ARGGYDYDGDY | 27 |
| 5A16A | GYTFTRYI | 28 | INPYNDGT | 29 | ARDSSGYGGAYAMDF | 30 |
| 14L22A | GFTFSSYA | 31 | INSNGGNT | 32 | ARHRGGFYYAVDY | 33 |
| 10P18A | GYSFTGYY | 34 | IYPSNGET | 35 | ARESYAMDY | 36 |
| 13P11A | GYSITNGNHW | 37 | ISSSGST | 38 | ATTGTWGYFDY | 39 |
| 3C16A | GYTFTSYV | 40 | VIPYNDGT | 41 | ARPSNWDEFDY | 42 |
| 3I21A | GYTFTNYW | 43 | IHPSDSET | 44 | ARYDGYFAY | 45 |
| 8H5A | GFSLSTFGMG | 46 | IWWDDDK | 47 | ARTYDYDEYFDY | 48 |
| 15K2A | GYTFTSYW | 49 | IHPSDSET | 50 | AREDGYYWYFDV | 51 |
| 5A24A | GYKFTDFN | 52 | INPNSGGT | 53 | ARWDYGNFAY | 54 |
| 15P17A | GYTFTNYW | 55 | IHPSDSET | 56 | AREDGYYWYFDV | 57 |
| 15N21A | GFTFSSYA | 58 | INSNGGRN | 59 | ARHRGGYYYAMDY | 60 |

HC: heavy chain;
CDR: complementarity determining region;
NO: SEQ ID NO

The HC CDRs for the antigen binding domains that specifically bind DLL3 were determined utilizing the IMGT method (Lefranc, M. -P. et al., Nucleic Acids Res. 1999; 27:209-212).

TABLE 4
CDR regions 1-3 of light chain for the antigen
binding domains that specifically bind DLL3

| | | | LC | | | |
|---|---|---|---|---|---|---|
| Name | LC CDR1 | NO | CDR2 | NO | LC CDR3 | NO |
| 13P9A | QNINVW | 61 | KAS | 62 | QQGQSYPFT | 63 |
| 5A16A | GNIHNY | 64 | NAK | 65 | QHFWFIPWT | 66 |
| 14L22A | QSVLYSSNQKNY | 67 | WAS | 68 | HQYLSSRT | 69 |
| 10P18A | KSVSTSGYSY | 70 | LAS | 71 | QHSRELPYT | 72 |
| 13P11A | ENVGTY | 73 | GAS | 74 | GQSYSYPFT | 75 |
| 3C16A | QNVRTA | 76 | LAS | 77 | LQHWNYPLT | 78 |
| 3I21A | DHINNW | 79 | GAT | 80 | QQYWSIPFT | 81 |
| 8H5A | KSLLHSNGITY | 82 | QMS | 83 | AQNLELPFT | 84 |
| 15K2A | ESVDIYGNSF | 85 | LAS | 86 | QQNNEDPWT | 87 |
| 5A24A | KSLLHSNGITY | 88 | QMS | 89 | AQNLELPLT | 90 |
| 15P17A | ESVDSYGNSF | 91 | LAS | 92 | QQNHEDPWT | 93 |
| 15N21A | QSLLYSSNQKNY | 94 | WAS | 95 | QQYYTYLT | 96 |

LC: light chain;
CDR: complementarity determining region;
NO: SEQ ID NO

The LC CDRs for the antigen binding domains that specifically bind DLL3 were determined utilizing the IMGT method (Lefranc, M. -P. et al., Nucleic Acids Res. 1999; 27:209-212).

TABLE 5
CDR regions 1-3 of heavy chain for the antigen binding
domains that specifically bind DLL3

| Name | HC CDR1 | NO | HC CDR2 | NO | HC CDR3 | NO |
|---|---|---|---|---|---|---|
| 13P9A | SYVMH | 97 | YINPYNDATKYNEKFKG | 98 | GGYDYDGDY | 99 |
| 5A16A | RYILH | 100 | YINPYNDGTKYNEKFKG | 101 | DSSGYGGAYAMDF | 102 |
| 14L22A | SYAMS | 103 | AINSNGGNTYYPDTVKD | 104 | HRGGFYYAVDY | 105 |
| 10P18A | GYYID | 106 | YIYPSNGETSYNQKFKG | 107 | ESYAMDY | 108 |
| 13P11A | NGNHWWS | 109 | YISSSGSTDSNPSLKS | 110 | TGTWGYFDY | 111 |
| 3C16A | SYVMH | 112 | YVIPYNDGTKYNEKFKG | 113 | PSNWDEFDY | 114 |
| 3I21A | NYWMN | 115 | RIHPSDSETHYNQKFKT | 116 | YDGYFAY | 117 |
| 8H5A | TFGMGVG | 118 | HIWWDDDKYYNPALKS | 119 | TYDYDEYFDY | 120 |
| 15K2A | SYWMN | 121 | RIHPSDSETHYNQKFRT | 122 | EDGYYWYFDV | 123 |
| 5A24A | DFNMD | 124 | DINPNSGGTIYNQKFKG | 125 | WDYGNFAY | 126 |
| 15P17A | NYWMN | 127 | RIHPSDSETHYNQKFKS | 128 | EDGYYWYFDV | 129 |
| 15N21A | SYAMS | 130 | AINSNGGRNYYPDTVKD | 131 | HRGGYYYAMDY | 132 |

HC: heavy chain;
CDR: complementarity determining region;
NO SEQ ID NO

The HC CDRs for the antigen binding domains that specifically bind DLL3 were determined utilizing the Kabat (Elvin A. Kabat et al, Sequences of Proteins of Immunological Interest 5th ed. 1991) method.

TABLE 6
CDR regions 1-3 of light chain for the antigen binding
domains that specifically bind DLL3

| Name | LC CDR1 | NO | LC CDR2 | NO | LC CDR3 | NO |
|---|---|---|---|---|---|---|
| 13P9A | HASQNINVWLS | 133 | KASNLHT | 134 | QQGQSYPFT | 135 |
| 5A16A | RASGNIHNYLA | 136 | NAKTLPY | 137 | QHFWTTPWT | 138 |
| 14L22A | KSSQSVLYSSNQKNYLA | 139 | WASTRES | 140 | HQYLSSRT | 141 |
| 10P18A | RASKSVSTSGYSYMH | 142 | LASNLES | 143 | QHSRELPYT | 144 |
| 13P11A | KASENVGTYVS | 145 | GASNRFT | 146 | GQSYSYPFT | 147 |
| 3C16A | KASQNVRTAVA | 148 | LASNRHT | 149 | LQHWNYPLT | 150 |
| 3I21A | KASDHINNWLA | 151 | GATSLET | 152 | QQYWSIPFT | 153 |
| 8H5A | RSSKSLLHSNGITYFY | 154 | QMSNLAS | 155 | AQNLELPFT | 156 |
| 15K2A | RASESVDIYGNSFMH | 157 | LASNLES | 158 | QQNNEDPWT | 159 |
| 5A24A | RSSKSLLHSNGITYLY | 160 | QMSNLAS | 161 | AQNLELPLT | 162 |
| 15P17A | RASESVDSYGNSFMH | 163 | LASNLES | 164 | QQNHEDPWT | 165 |
| 15N21A | KSSQSLLYSSNQKNYLA | 166 | WASTRES | 167 | QQYYTYLT | 168 |

LC: light chain;
CDR: complementarity determining region;
NO: SEQ ID NO

The LC CDRs for the antigen binding domains that specifically bind DLL3 were determined utilizing the Kabat (Elvin A. Kabat et al, Sequences of Proteins of Immunological Interest 5th ed. 1991) method.

### Example 2: Humanization of Mouse Anti-DLL3 mAbs

The mouse anti-DLL3 mAbs were humanized to reduce the potential of immunogenicity when used in human patients as described in PCT Patent Application No. PCT/US2019/029888, filed on Apr. 30, 2019, which is incorporated herein by reference in its entirety. The sequences of the humanized VH and VL regions are shown in Tables 7 and 8. The humanized VH and VL were named as follows: 13P9-H1 refers to the H1 sequence of humanized VH for mouse mAb 13P9A; 13P9-L1 refers to the L1 sequence of humanized VL for mouse mAb 13P9A. All the other humanized VH and VL regions adopt the same naming rule.

TABLE 7
Sequences of the humanized heavy chain variable region of
anti-DLL3 mAb

| | | SEQ |
|---|---|---|
| | | ID |
| Design | VH | NO: |
| 13P9-H1 | EVRLSQSGGQMKKPGESMRLSCRASGYTFTSYVMHWVRQAPGRRPEWIGYINPY | 170 |
| | NDATKYARKFQGRATLTSDKYSDTAFLELRSLTSDDTAVYYCARGGYDYDGDYW | |
| | GRGAPVTVSS | |
| 5A16-H1 | QVQLVQSGAEVKKPGSSVKVSCKASGYTFTRYILHWVRLAPGQGLEWIGYINPYN | 175 |
| | DGTKYNEKFKGKATLTSDKSTNTAYMELSSLRSEDTAVYYCARDSSGYGGAYAM | |
| | DFWGQGTLVTVSS | |
| 5A16-H2 | QVQLVQSGAEVKKPGASVKVSCKASGYTFTRYILHWVRLAPGQGLEWIGYINPYN | 176 |
| | DGTKYNQKFKGKATLTSDKSTNTAYMELSSLRSEDTAVYYCARDSSGYGGAYAM | |
| | DFWGQGTLVTVSS | |
| 5A16-H3 | QVQLVQSGAEVKKPGESVKVSCKASGYTFTRYILHWVRLAPGQGLEWIGWINPYN | 177 |
| | DGTQYNEKFKGRATLTSDKSTSTAYMELSSLRSEDTAVYYCARDSSGYGGAYAM | |
| | DFWGQGTTVTVSS | |
| 5A16-H4 | QVQLVQSGAEVKKPGASVKVSCKASGYTFTRYILHWVRLAPGQGLEWIGWINPYN | 178 |
| | DGTQYNEKFKGRATLTSDTSTSTAYMELSSLRSEDTAVYYCARDSSGYGGAYAM | |
| | DFWGQGTTVTVSS | |
| 10P18-H1 | QVQLVQSGAEVKKPGSSVKVSCKASGYSFTGYYIDWVRQAPGQGLEWIGYIYPSN | 179 |
| | GETSYNQKFKGRATLTVDKSTSTVYMELSSLRSEDTAVYYCARESYAMDYWGQG | |
| | TLVTVSS | |
| 10P18-H2 | QVQLVQSGAEVKKPGASVKVSCKASGYSFTGYYIDWVRQAPGQGLEWIGYIYPSN | 180 |
| | GETSYNQKFKGRATLTVDTSTSTVYMELSSLRSEDTAVYYCARESYAMDYWGQG | |
| | TTVTVSS | |
| 10P18-H3 | EVQLVESGGGLVQPGGSLRLSCAASGYSFTGYYMDWVRQAPGKGLEWIGDIYPSN | 181 |
| | GETIYNQEFKGRATLSVDKSKNTVYLQMNSLRAEDTAVYYCARESYAMDYWGQ | |
| | GTLVTVSS | |
| 10P18-H4 | EVQLVESGGGLVQPGGSLRLSCAASGYSFTGYYMSWVRQAPGKGLEWIGDIYPSN | 182 |
| | GETIYNQSFKGRATLSVDNSKNTLYLQMNSLRAEDTAVYYCARESYAMDYWGQG | |
| | TLVTVSS | |
| 3C16-H1 | QVQLVQSGAEVKKPGSSVKVSCKASGYTFTSYVLHWVRQAPGQGLEWIGWVIPY | 183 |
| | NDGTQYNEKFKGRATLTSDKSTSTAYMELSSLRSEDTAVYYCARPSNWDEFDYW | |
| | GQGTTVTVSS | |
| 3C16-H2 | QVQLVQSGAEVKKPGASVKVSCKASGYTFTSYVLHWVRQAPGQGLEWIGWVIPY | 184 |
| | NDGTKYNEKFKGRATLTSDKSTSTAYMELSSLRSEDTAVYYCARPSNWDEFDYW | |
| | GQGTLVTVSS | |
| 3C16-H3 | EVQFVQSGAEVKKPGASVRVSCEASGYTFTSYVLQWVRQAPGQRLEWIGWVIPYN | 185 |
| | DGTSYAPQFQGRATLTSDKYTSTAYMHFKNLRSDDTAIYYCARPSNWDEFDYWG | |
| | QGTLVTVSS | |
| 3I21-H1 | EVTLKESGPTLVKPTQTLTLTCTASGYTFTNYWVSWVRQPPGKALEWIGHIHPSDS | 186 |
| | ETRYNPSLKSRATLTVDKSKNQAVLTMTNMDPVDTATYYCARYDGYFAYWGQG | |
| | TLVTVSS | |
| 3I21-H2 | QVTLKESGPALVKPTQTLTLTCTASGYTFTNYWVSWVRQPPGKALEWIGHIHPSDS | 187 |
| | ETRYNPSLKSRATLTVDTSKNQAVLTMTNMDPVDTATYYCARYDGYFAYWGQG | |
| | TLVTVSS | |
| 3I21-H3 | EVQLLESGGGLVQPGGSLRLSCAASGYTFTNYWMSWVRQAPGKGLEWIGAIHPSD | 188 |
| | SETYYADSVKGRATLSVDKSKNTAYLQMNSLRAEDTAVYYCARYDGYFAYWGQ | |
| | GTLVTVSS | |
| 3I21-H4 | EVQLLESGGGLVQPGGSLRLSCAASGYTFTNYWMSWVRQAPGKGLEWIGAIHPSD | 189 |
| | SETYYADSVKGRATLSVDNSKNTAYLQMNSLRAEDTAVYYCARYDGYFAYWGQ | |
| | GTLVTVSS | |
| 3I21-H5 | EVQLLESGGGLVQPGGSLRLSCAASGYTFTNYWMSWVRQAPGKGLEWIGAIHPSD | 190 |
| | SETYYADSVKGRATLSVDNSKNTAYLQMNSLRAEDTAVYYCARYDAYFAYWGQ | |
| | GTLVTVSS | |
| 15K2-H1 | EVQLVESGGGLVQPGRSLRLSCAASGYTFTSYWMHWMRQAPGKGLEWIGGIHPS | 191 |
| | DSETGYADSVKGRATLSVDKAKNSAYLQMNSLRAEDMALYYCAREDGYYWYFD | |
| | VWGQGTMVTVSS | |
| 15K2-H2 | EVQLVQSGAEVKKPGESLKISCRASGYTFTSYWIGWMRQMPGKGLEWIGIIHPSDS | 192 |
| | ETRYSPSFQGQATLSVDKSINTAYLQWSSLKASDTAMYYCAREDGYYWYFDVWG | |
| | QGTLVTVSS | |
| 15K2-H3 | QVQLVQSGAEVKKPGSSVKVSCKASGYTFTSYWISWMRQAPGQGLEWIGSIHPSD | 193 |
| | SETNYAQKFQGRATLTVDKSTSTAYMELSSLRSEDTAVYYCAREDGYYWYFDVW | |
| | GQGTLVTVSS | |
| 5A24-H1 | QVQLVQSGAEVKKPGASVKVSCKASGYKFTDFNMHWVRQAPGQGLEWIGNINPN | 194 |
| | SGGTNYAEKFKNRATLTVDKSISTAYMELSRLRSDDTAVYYCARWDYGNFAYWG | |
| | QGTLVTVSS | |
| 5A24-H2 | QVQLVQSGAEVKKPGASVKVSCKASGYKFTDFNMDWVRQAPGQGLEWIGNINPN | 195 |
| | SGGTNYAEKFKNRATLTVDTSISTAYMELSRLRSDDTAVYYCARWDYGNFAYWG | |
| | QGTLVTVSS | |
| 5A24-H3 | QVQLVQSGAEVKKPGASVKVSCKASGYKFTDFNMHWVRQAPGQGLEWIGEINPN | 196 |
| | SGGTTYNEKFKGKATLTVDKSTSTAYMELSSLRSEDTAVYYCARWDYGNFAYWG | |
| | QGTLVTVSS | |
| 5A24-H4 | QVQLVQSGAEVKKPGSSVKVSCKASGYKFTDFNMHWVRQAPGQGLEWIGYINPN | 197 |
| | SGGTEYNQKFKDKATLTVDKSTNTAYMELSSLRSEDTAVYYCARWDYGNFAYW | |
| | GQGTLVTVSS | |
| 15P17-H1 | EVQLVQSGAEVKKPGSSVKVSCKASGYTFTNYWISWVRQAPGQGLEWIGSIHPSD | 198 |
| | SETNYAQKFQGRATLTVDKSTSTAYMELSSLRSEDTAVYYCAREDGYYWYFDVW | |
| | GQGTLVTVSS | |
| 15P17-H2 | EVQLVQSGAEVKKPGSSVKVSCKASGYTFTNYWISWVRQAPGQGLEWIGSIHPSD | 199 |
| | SETNYAQKFkGRATLTVDKSTSTAYMELSSLRSEDTAVYYCAREDGYYWYFDVW | |
| | GQGTLVTVSS | |
| 15P17-H3 | EVTLKESGPTLVKPTQTLTLTCTASGYTFWVRQPPGKALEWIGRIHPSDS | 200 |
| | ETHYNQKFKSRATLTVDKSKNQAVLTMTNMDPVDTATYYCAREDGYYWYFDVW | |
| | GQGTLVTVSS | |
| 15P17-H4 | EVTLKESGPTLVKPTQTLTLTCTASGYTFWVRQPPGKALEWIGRIHPSDS | 201 |
| | ETHYNQKFKSRATLTVDTSKNQAVLTMTNMDPVDTATYYCAREDGYYWYFDVW | |
| | GQGTLVTVSS | |
| 15N21-H1 | EVQLLESGGGLVQPGGSLRLSCAASGFTFSSYAMSWVRQAPGKGLEWVAAINSNG | 202 |
| | GRNYYADSVKGRFTISRDNSKNTLYLQMNSLRAEDTAVYYCARHRGGYYYAMD | |
| | YWGQGTLVTVSS | |
| 15N21-H2 | EVQLLESGGGLVQPGGSLRLSCAASGFTFSSYAMSWVRQAPGKGLEWVAAINSNG | 203 |
| | GRNYYPDSVKGRFTISRDNSKNTLYLQMNSLRAEDTAVYYCARHRGGYYYAMDY | |
| | WGQGTLVTVSS | |
| 15N21-H3 | EVQLVESGGGLVQPGGSLRLSCVASGFTFSSYAMSWVRQAPGKGLEWVASINSNG | 204 |
| | GRNYYSDSVKGRFTISRDNSKNTLYLQMNSLRAEDTAVYYCARHRGGYYYAMDY | |
| | WGQGTLVTVSS | |
| 8H5-H1 | EVTLKESGPTLVKPTQTLTLTCTFSGFSLSTFGMGVSWIRQPPGKALEWLAHIWWD | 205 |
| | DDKRYNPSLKSRLTITKDTSKNQVVLTMTNMDPVDTATYYCARTYDYDEYFDYW | |
| | GQGTLVTVSS | |
| 8H5-H2 | QVTLKESGPTLVKPTQTLTLTCTFSGFSLSTFGMGVGWIRQPPGKALEWLAHIWW | 206 |
| | DDDKRYNPSLKSRLTITKDTSKNQVVLTMTNMDPVDTATYYCARTYDYDEYFDY | |
| | WGQGTLVTVSS | |
| 8H5-H3 | EVTLKESGPVLVKPTETLTLTCTFSGFSLSTFGMGVGWIRQPPGKALEWLAHIWWD | 207 |
| | DDKRYNPALKSRLTISKDTSKSQVVLTMTNMDPVDTATYYCARTYDYDEYFDYW | |
| | GQGTLVTVSS | |
| 14L22-H1 | EVQLLESGGGLVQPGGSLRLSCAASGFTFSSYAMSWVRQAPGKGLEWVAAINSNG | 208 |
| | GNTYYADSVKGRFTISRDNSKNTLYLQMNSLRAEDTAVYYCARHRGGFYYAVDY | |
| | WGQGTLVTVSS | |
| 14L22-H2 | QVELVESGGGLVQPGGSLRLSCAASGFTFSSYAMSWVRQAPGKGLEWVAAINSNG | 209 |
| | GNTYYADSVKGRFTISRDNSKNTLYLQMNSLRAEDTAVYYCARHRGGFYYAVDY | |
| | WGQGTLVTVSS | |
| 3I21-H6 | EVTLKESGPTLVKPTQTLTLTCKASGYTFWVRQPPGKALEWIGRIHPSD | 248 |
| | SETHYNPSLKSRATLTVDKSKNQAVLTMTNMDPVDTATYYCARYDGYFAYWGQ | |
| | GTLVTVSS | |
| 3I21-H7 | EVQLLESGGGLVQPGGSLRLSCKASGYTFTNYWMNWVRQAPGKGLEWIGRIHPSD | 249 |
| | SETHYNDSVKGRATLSVDKSKNTAYLQMNSLRAEDTAVYYCARYDGYFAYWGQ | |
| | GTLVTVSS | |
| 15K2-H4 | EVQLVESGGGLVQPGRSLRLSCKASGYTFTSYWMNWMRQAPGKGLEWIGRIHPS | 250 |
| | DSETHYNDSVKGRATLSVDKAKNSAYLQMNSLRAEDMALYYCAREDGYYWYFD | |
| | VWGQGTMVTVSS | |
| 15K2-H5 | EVQLVQSGAEVKKPGESLKISCKASGYTFTSYWMNWMRQMPGKGLEWIGRIHPS | 251 |
| | DSETHYNPSFQGQATLSVDKSINTAYLQWSSLKASDTAMYYCAREDGYYWYFDV | |
| | WGQGTLVTVSS | |
| 15K2-H6 | QVQLVQSGAEVKKPGSSVKVSCKASGYTFTSYWMNWMRQAPGQGLEWIGRIHPS | 252 |
| | DSETHYNQKFQGRATLTVDKSTSTAYMELSSLRSEDTAVYYCAREDGYYWYFDV | |
| | WGQGTLVTVSS | |
| 5A24-H5 | QVQLVQSGAEVKKPGASVKVSCKASGYKFTDFNMDWVRQAPGQGLEWIGDINPN | 253 |
| | SGGTIYNEKFKNRATLTVDKSISTAYMELSRLRSDDTAVYYCARWDYGNFAYWG | |
| | QGTLVTVSS | |
| 8H5-H4 | EVTLKESGPTLVKPTQTLTLTCSFSGFSLSTFGMGVGWIRQPPGKALEWLAHIWWD | 254 |
| | DDKYYNPSLKSRLTITKDTSKNQVVLTMTNMDPVDTATYYCARTYDYDEYFDYW | |
| | GQGTLVTVSS | |
| 8H5-H5 | EVTLKESGPVLVKPTETLTLTCSFSGFSLSTFGMGVGWIRQPPGKALEWLAHIWWD | 255 |
| | DDKYYNPALKSRLTISKDTSKSQVVLTMTNMDPVDTATYYCARTYDYDEYFDYW | |
| | GQGTLVTVSS | |

TABLE 8
Sequences of humanized light chain variable regions of anti-DLL3 mAb

| | | SEQ |
|---|---|---|
| | | ID |
| Design | VL | NO: |
| 13P9-L1 | EIVMTQSPGTLSLSPGERATLSCHASQNINVWLSWYQQKPGQAPRLLIYKASNLHTG | 171 |
| | IPDRFSGSGSGTDFTLTISRLEPEDFAVYYCQQGQSYPFTFGQGTKVEIK | |
| 13P9-L2 | EIVLTQSPGTLSLSPGERATLSCHASQNINVWLSWYQQKPGQAPRLLIYKASNLHTGI | 172 |
| | PDRFSGSGSGTDFTLTISRLEPEDFAVYYCQQGQSYPFTFGQGTKVEIK | |
| 13P9-L3 | EIVMTQSPATLSLSPGETAIISCHASQNINVWLSWYQQRPGQAPRLLIYKASNLHTGI | 173 |
| | PDRFSGSGWGTDFNLSISNLESGDFGVYYCQQGQSYPFTFGQGTKVEIK | |
| 13P9-L4 | EIVMTQSPATLSLSPGETAIISCHASQNINVWLSWYQQRPGQAPRLLIYKASNLHTGI | 174 |
| | PDRFSGSGWGTDFNLSISNLESGDFGVYYCQQGQSYPWTFGQGTKVEIK | |
| 5A16-L1 | DIQMTQSPSTLSASVGDRVTITCRASGNIHNYLAWYQQKPGKAPKLLVYNAKTLPY | 210 |
| | GVPARFSGSGSGTEYTLTISSLQPDDFATYYCQHFWTTPWTFGQGTKVEVK | |
| 5A16-L2 | DIQMTQSPSTLSASVGDRVTITCRASGNIHNYLAWYQQKQGKAPKLLVYNAKTLPY | 211 |
| | GVPARFSGSGSGTEYTLTISSLQPDDFATYYCQHFWTTPWTFGGGTKVEVK | |
| 5A16-L3 | DIQMTQSPSSLSASVGDRVTITCKASGNIHNYLAWYQQKPGKAPKLLVYNAKYRYS | 212 |
| | GVPSRFSGSGSGTDYTLTISSLQPEDFATYYCQHFWTTPWTFGQGTKVEIK | |
| 10P18-L1 | DIQLTQSPSSVSASVGDRVTITCRASKSVSTSGYSYMHWYQQKPGKAPKLLIYLASN | 213 |
| | LESGVPSRFSGSGSGTDFTLTISSLQPEDFATYYCQHSRELPYTFGQGTKVEIK | |
| 10P18-L2 | DIVLTQSPDSLAVSLGERATINCRASKSVSTSGYSYLAWYQQKPGQPPKLLIYLASNL | 214 |
| | ESGVPDRFSGSGSGTDFTLTISSLQAEDVAVYYCQHSRELPYTFGGGTKVEIK | |
| 10P18-L3 | RIQLTQSPSSLSASVGDRVTITCKASKSVSTSGYSYVHWYQQKPGKAPKLLIYLASY | 215 |
| | RYTGVPSRFSGSGSGTDFTLTISSLQPEDFATYYCQHSRELPYTFGQGTKVEIK | |
| 3C16-L1 | DIQMTQSPSSLSASVGDRVTITCKASQNVRTAVAWYQQKPGKAPKALIYLASYRYS | 216 |
| | GVPSRFSGSGSGTDFTLTISSLQPEDFATYFCLQHWNYPLTFGQGTKVEIK | |
| 3C16-L2 | DIQMTQSPSSLSASVGDRVTITCKASQNVRTALAWYQQKPGKAPKALIYLASNRYS | 217 |
| | GVPSRFSGSGSGTDFTLTISSLQPEDFATYFCLQHWNYPLTFGQGTKVEIK | |
| 3C16-L3 | EIVMTQSPVTVSVSRGGTATLSCRASQNVRTAVAWYQQKPGQTPRALIYLASTRAS | 218 |
| | GVPERFSGSGFGTDFTLSISGLQPEDVAIYFCLQHWNYPLTFGQGTKVEIK | |
| 3I21-L1 | DIVMTQSPDSLAVSLGERATINCRASDHINNWMAWYQQKPGQPPKLLISGATNPES | 219 |
| | GVPDRFSGSGSGKDYTLTISSLQAEDVAVYYCQQYWSIPFTFGQGTKVEIK | |
| 3I21-L2 | DIVMTQSPDSLAVSLGERATINCKASDHINNWLAWYQQKPGQPPKLLISGATTRESG | 220 |
| | VPDRFSGSGSGKDYTLTISSLQAEDVAVYYCQQYWSIPFTFGQGTRLEIK | |
| 15K2-L1 | DIQLTQSPSTLSASVGDRVTITCRASESVDIYGNSFLHWYQQKPGKVPKLLIYLASSL | 221 |
| | ESGVPSRFSGSGSRTEFTLTISSLQPDDFATYYCQQNNEDPWTFGPGTKVDIK | |
| 15K2-L2 | DIQLTQSPSTLSASVGDRVTITCRASESVDIYGNSFLAWYQQKPGKVPKLLIYLASSL | 222 |
| | ESGVPSRFSGSGSGTEFTLTISSLQPDDFATYYCQQNNEDPWTFGPGTKVDIK | |
| 15K2-L3 | DIQLTQSPSSLSASVGDRVTITCRASESVDIYGNSFLHWYQQKPGKTPKLLIYLASSL | 223 |
| | QSGVPSRFSGSGSRTDFTLTISSLQPEDFATYYCQQNNEDPWTFGQGTKVEIK | |
| 15K2-L4 | DIQLTQSPSSLSASVGDRVTITCRASESVDIYGNSFLHWYQQKPGKTPKLLIYLASSL | 224 |
| | QSGVPSRFSGSGSGTDFTLTISSLQPEDFATYYCQQNNEDPWTFGQGTKVEIK | |
| 5A24-L1 | DIVMTQSPLSLPVTPGEPASISCRSSKSLLHSNGITYLYWYLQKPGQSPQLLIYQMSD | 225 |
| | RFSGVPDRFSSSGSGTDFTLKISRVEAEDVGVYYCAQNLELPLTFGQGTKVEIK | |
| 5A24-L2 | DIVMTQTPLSLSVTPGQPASISCRSSKSLLHSNGITYLYWYLQKPGQSPKLLIYQMSY | 226 |
| | RFSGVPDRFSSSGSGTDFTLKISRVEAEDVGVYYCAQNLELPLTFGQGTKLEIK | |
| 5A24-L3 | DIQMTQSPSTLSASVGDRVTITCSSSKSLLHSNGITYMYWYQQKPGKAPKLLIYQMS | 227 |
| | NLASGVPARFSSSGSGTEFTLTISSLQPDDFATYYCAQNLELPLTFGQGTKVEVK | |
| 5A24-L4 | DIQMTQSPSTLSASVGDRVTITCSSSKSLLHSNGITYLAWYQQKPGKAPKLLIYQMS | 228 |
| | NLASGVPARFSSSGSGTEFTLTISSLQPDDFATYYCAQNLELPLTFGQGTKVEVK | |
| 15P17-L1 | DIQLTQSPSSVSASVGDRVTITCRASESVDSYGNSFLHWYQQKPGKAPKLLIYLASSL | 229 |
| | QSGVPSRFSGSGSRTDFTLTISSLQPEDFATYYCQQNHEDPWTFGQGTKVEIK | |
| 15P17-L2 | DIVLTQSPDSLAVSLGERATINCRASESVDSYGNSFMHWYQQKPGQPPKLLIYLASN | 230 |
| | LESGVPDRFSGSGSRTDFTLTISSLQAEDVAVYYCQQNHEDPWTFGQGTKVEIK | |
| 15P17-L3 | DIVLTQSPDSLAVSLGERATINCRASESVDSYGNSFMHWYQQKPGQPPKLLIYLASN | 231 |
| | LESGVPDRFSGSGSGTDFTLTISSLQAEDVAVYYCQQNHEDPWTFGQGTKVEIK | |
| 15N21-L1 | DIVMTQSPDSLAVSLGERATINCKSSQSLLYSSNQKNYLAWYQQKPGQPPKLLIYW | 232 |
| | ASTRESGVPDRFSGSGSGTDFTLTISSLQAEDVAVYYCQQYYTYLTFGQGTKVEIK | |
| 15N21-L2 | DIVMTQSPDSLAVSLGERATINCKSSQSLLYSSNQKNYLAWYQQKPGQPPKLLIYW | 233 |
| | ASTRESGVPDRFSGSGSGTDFTLTISSLQAEDVAVYYCQQYYTYLTFGQGTRLEIK | |
| 15N21-L3 | DIVMTQSPATLSLSPGERATLSCMSSQSLLYSSNQKNYMAWYQQKPGQAPRLLIYW | 234 |
| | ASTRAPGVPARFSGSGSGTDFTLTISSLEPEDFAVYYCQQYYTYLTFGQGTKVEIK | |
| 8H5-L1 | DIVMTQSPDSLAVSLGERATINCRSSKSLLHSNGITYMYWYQQKPGQPPKLLIYQMS | 235 |
| | NPESGVPDRFSSSGSGTDFTLTISSLQAEDVAVYYCAQNLELPFTFGQGTKVEIK | |
| 8H5-L2 | DIVMTQSPDSLAVSLGERATINCKSSKSLLHSNGIQQKPGQPPKLLIYQMS | 236 |
| | NPESGVPDRFSGSGSGTDFTLTISSLQAEDVAVYYCAQNLELPFTFGQGTKVEIK | |
| 8H5-L3 | DIVMTQSPDSLAVSLGERATINCKSSKSLLHSNGIQQKPGQPPKLLIYQMS | 237 |
| | TRESGVPDRFSGSGSGTDFTLTISSLQAEDVAVYYCAQNLELPFTFGQGTKVEIK | |
| 8H5-L4 | EIVMTQSPATLSLSPGERATLSCRSSKSLLHSNGITYLYWYQQKPGQAPRLLIYQMST | 238 |
| | LQSGIPARFSSSGSGTDFTLTISSLEPEDFAVYYCAQNLELPFTFGQGTKLEIK | |
| 14L22-L1 | DIVMTQSPDSLAVSLGERATINCKSSQSVLYSSNQKNYLAWYQQKPGQPPKLLIYW | 239 |
| | ASTRESGVPDRFSGSGSGTDFTLTISSLQAEDVAVYYCHQYLSSRTFGQGTRLEIK | |
| 14L22-L2 | DIVLTQSPATLSLSPGERATLSCRSSQSVLYSSNQKNYLAWYQQKPGQAPRLLIYWA | 240 |
| | SSRATGVPARFSGSGSGTDFTLTISSLEPEDFATYYCHQYLSSRTFGQGTKVEIK | |
| 3I21-L3 | DIVMTQSPDSLAVSLGERATINCRASDHINNWLAWYQQKPGQPPKLLISGATSLESG | 256 |
| | VPDRFSGSGSGKDYTLTISSLQAEDVAVYYCQQYWSIPFTFGQGTKVEIK | |
| 3I21-L4 | DIVMTQSPDSLAVSLGERATINCRASDHINNWLAWYQQKPGQPPKLLISGATSLESG | 257 |
| | VPDRFSGSGSGKDYTLTISSLQAEDVAVYYCQQYWSIPFTFGQGTRLEIK | |
| 15K2-L5 | DIQLTQSPSTLSASVGDRVTITCRASESVDIYGNSFMHWYQQKPGKVPKLLIYLASN | 258 |
| | LESGVPSRFSGSGSRTEFTLTISSLQPDDFATYYCQQNNEDPWTFGPGTKVDIK | |
| 15K2-L6 | DIQLTQSPSSLSASVGDRVTITCRASESVDIYGNSFMHWYQQKPGKTPKLLIYLASNL | 259 |
| | ESGVPSRFSGSGSRTDFTLTISSLQPEDFATYYCQQNNEDPWTFGQGTKVEIK | |
| 5A24-L5 | DIVMTQSPLSLPVTPGEPASISCRSSKSLLHSNGITYLYWYLQKPGQSPQLLIYQMSN | 260 |
| | LASGVPDRFSSSGSGTDFTLKISRVEAEDVGVYYCAQNLELPLTFGQGTKVEIK | |
| 5A24-L6 | DIVMTQTPLSLSVTPGQPASISCRSSKSLLHSNGITYLYWYLQKPGQSPKLLIYQMSN | 261 |
| | LASGVPDRFSSSGSGTDFTLKISRVEAEDVGVYYCAQNLELPLTFGQGTKLEIK | |
| 5A24-L7 | DIQMTQSPSTLSASVGDRVTITCRSSKSLLHSNGITYLYWYQQKPGKAPKLLIYQMS | 262 |
| | NLASGVPARFSSSGSGTEFTLTISSLQPDDFATYYCAQNLELPLTFGQGTKVEVK | |
| 8H5-L5 | DIVMTQSPDSLAVSLGERATINCRSSKSLLHSNGITYFYWYQQKPGQPPKLLIYQMS | 263 |
| | NLASGVPDRFSSSGSGTDFTLTISSLQAEDVAVYYCAQNLELPFTFGQGTKVEIK | |
| 8H5-L6 | EIVMTQSPATLSLSPGERATLSCRSSKSLLHSNGITYFYWYQQKPGQAPRLLIYQMSN | 264 |
| | LASGIPARFSSSGSGTDFTLTISSLEPEDFAVYYCAQNLELPFTFGQGTKLEIK | |

The humanized VH and VL regions were fused to the constant regions of human IgG1 heavy chain and kappa light chain, respectively. The humanized mAbs were named as follows: 13P9-H1L1 refers to the mAb with the 13P9-H1 heavy chain variable region and the 13P9-L1 light chain variable region; all the other humanized mAbs adopt the same naming rule. The chimeric antibodies were made by fusing the VH and VL regions of the mouse antibodies to the constant regions of human IgG1 heavy chain and kappa light chain, respectively. 3C16 refers to the chimeric antibody made using 3C16A; all the other chimeric mAbs adopt the same naming rule.

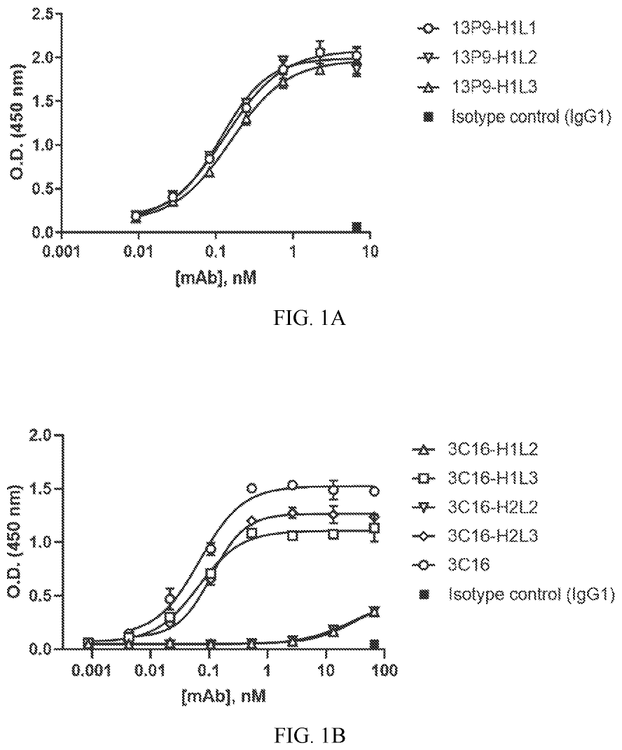

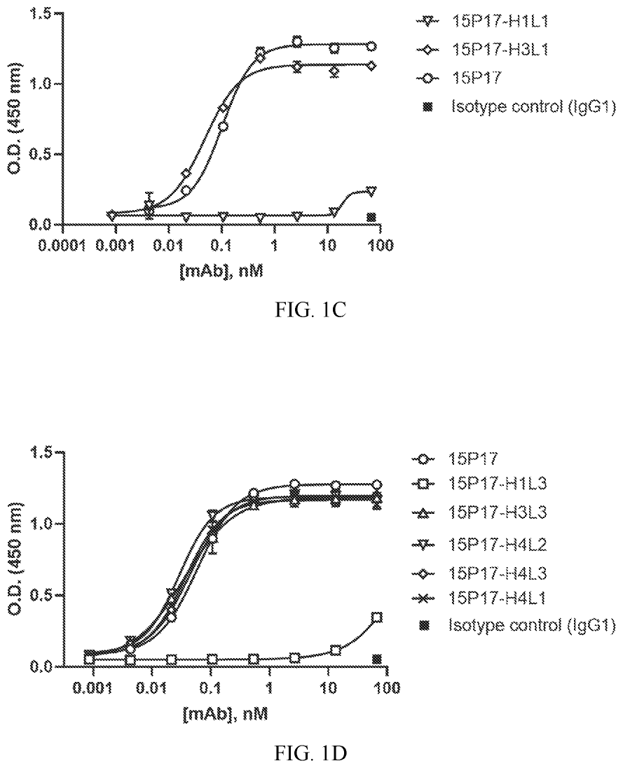

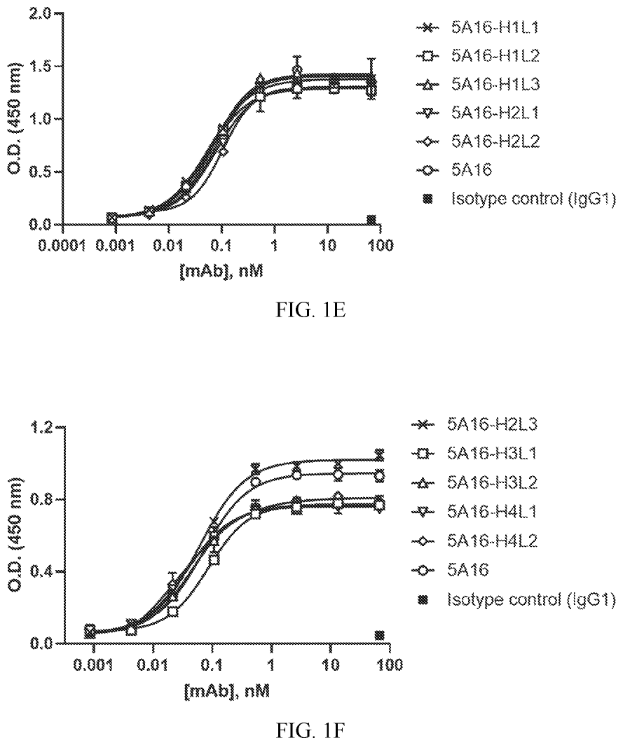

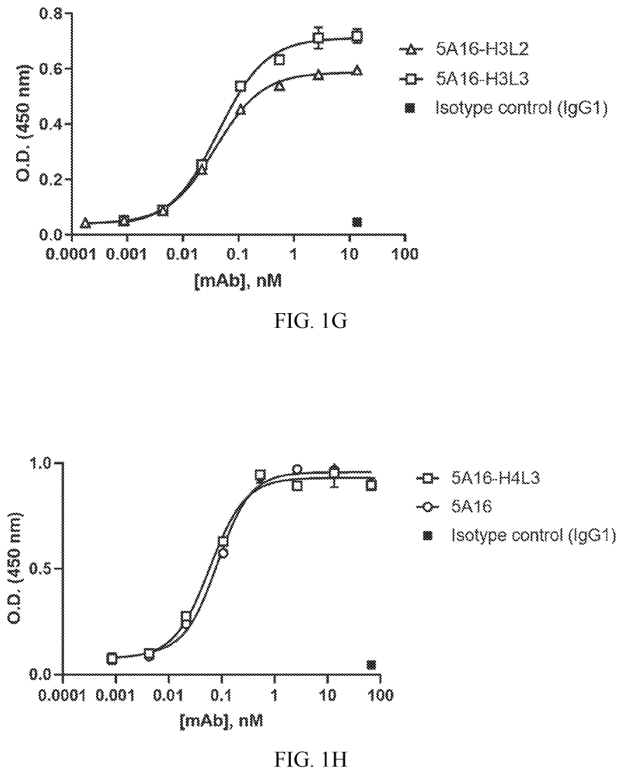

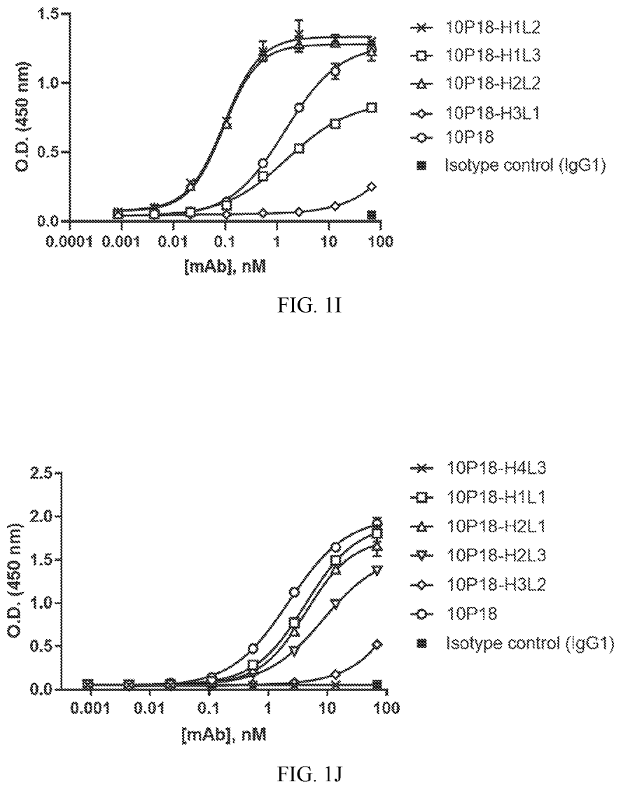

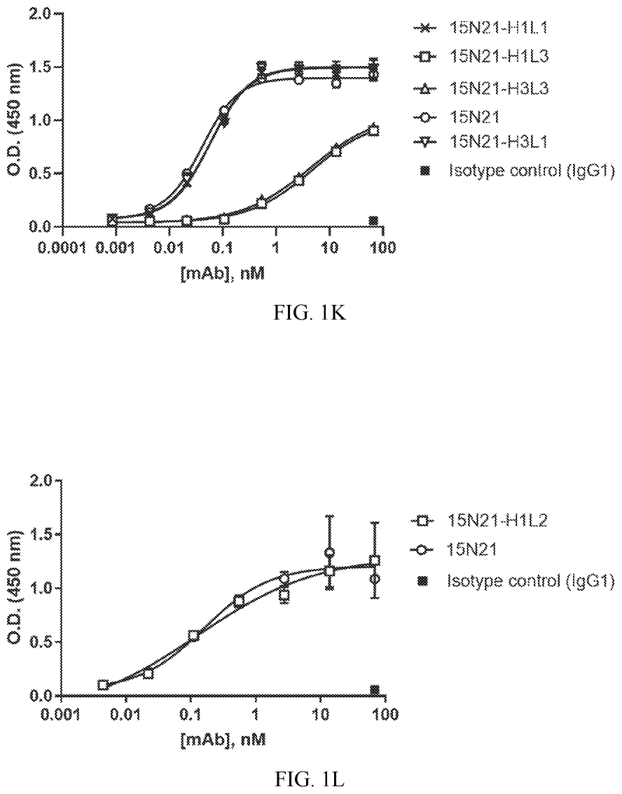

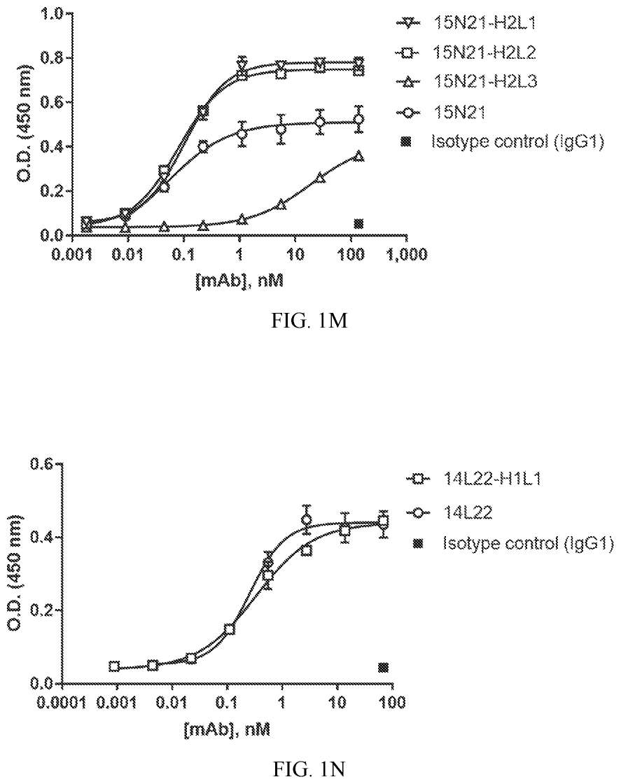

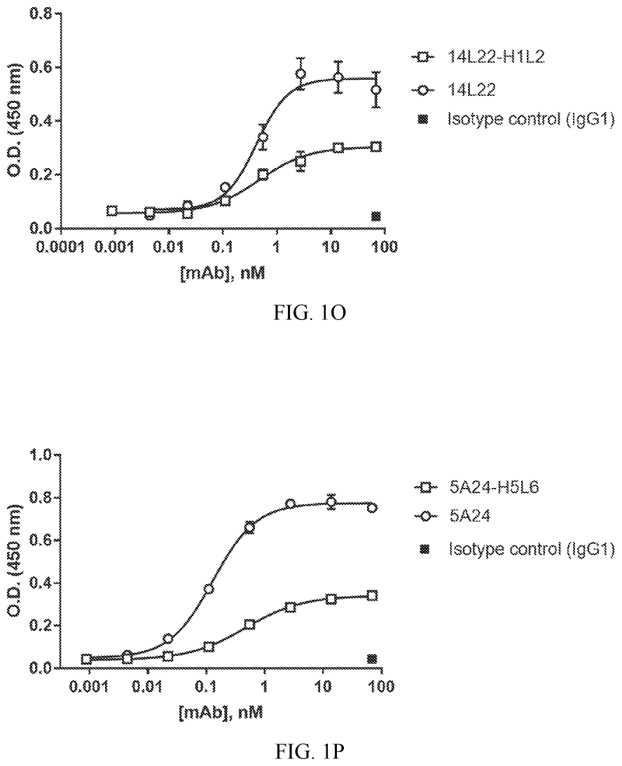

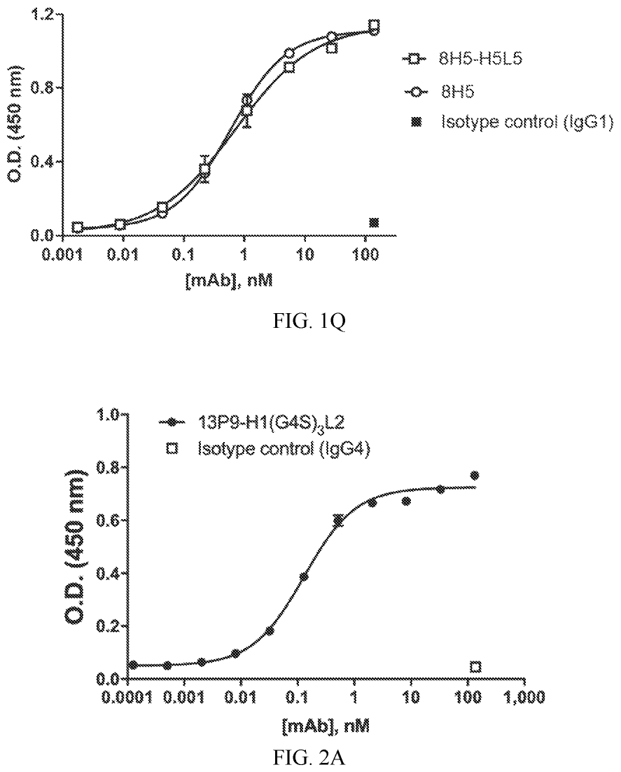

Humanized mAbs were tested for their ability to bind DLL3 in an ELISA assay. The results are shown in FIG. 1A-1Q.

### Example 3: Conversion of Chimeric and Humanized mAbs to Single Chain Variable Fragments (scFvs)

The chimeric and humanized mAbs were converted to scFvs, each of which consists of one VH and one VL with a (G₄S)ₙ linker in between (where “n” represents the number of the G₄S repeats). Either the VH or the VL region was placed at the N-terminus of the fusion protein to identify the most effective scFv designs. The sequences of the designed scFvs are shown in Table 9. The scFvs were named as following: 13P9-H1(G₄S)₃L2 refers to the scFv with 13P9-H1 heavy chain variable region, the (G₄S)₃ linker and 13P9-L2 light chain variable region; 5A16-H(G₄S)₃L refers to the scFv with 5A16A heavy chain variable region, the (G₄S)₃ linker and 5A16A light chain variable region; all the other scFvs adopt the same naming rule.

TABLE 9
Sequences of humanized scFvs that specifically bind DLL3

| | | SEQ |
|---|---|---|
| | | ID |
| Name | SEQUENCE | NO: |
| 13P9- | EVRLSQSGGQMKKPGESMRLSCRASGYTFTSYVMHWVRQAPGRRPEWIGYINP | 241 |
| H1(G₄S)₃L2 | YNDATKYARKFQGRATLTSDKYSDTAFLELRSLTSDDTAVYYCARGGYDYDGD | |
| | YWGRGAPVTVSSGGGGSGGGGSGGGGSEIVLTQSPGTLSLSPGERATLSCHASQN | |
| | INVWLSWYQQKPGQAPRLLIYKASNLHTGIPDRFSGSGSGTDFTLTISRLEPEDFA | |
| | VYYCQQGQSYPFTFGQGTKVEIK | |
| 13P9- | EVRLSQSGGQMKKPGESMRLSCRASGYTFTSYVMHWVRQAPGRRPEWIGYINP | 242 |
| H1(G₄S)₄L2 | YNDATKYARKFQGRATLTSDKYSDTAFLELRSLTSDDTAVYYCARGGYDYDGD | |
| | YWGRGAPVTVSSGGGGSGGGGSGGGGSGGGGSEIVLTQSPGTLSLSPGERATLSC | |
| | HASQNINVWLSWYQQKPGQAPRLLIYKASNLHTGIPDRFSGSGSGTDFTLTISRLE | |
| | PEDFAVYYCQQGQSYPFTFGQGTKVEIK | |
| 13P9- | EIVLTQSPGTLSLSPGERATLSCHASQNINVWLSWYQQKPGQAPRLLIYKASNLH | 243 |
| L2(G₄S)₃H1 | TGIPDRFSGSGSGTDFTLTISRLEPEDFAVYYCQQGQSYPFTFGQGTKVEIKGGGG | |
| | SGGGGSGGGGSEVRLSQSGGQMKKPGESMRLSCRASGYTFTSYVMHWVRQAPG | |
| | RRPEWIGYINPYNDATKYARKFQGRATLTSDKYSDTAFLELRSLTSDDTAVYYC | |
| | ARGGYDYDGDYWGRGAPVTVSS | |
| 5A16- | EVQLQQSGPELVKPGASVKMSCKASGYTFTRYILHWVKLKPGQGLEWIGYINPY | 244 |
| H(G₄S)₃L | NDGTKYNEKFKGKATLTSDKSSSTAYMELSRLTSYDSAVYYCARDSSGYGGAY | |
| | AMDFWGQGTSVTVSSGGGGSGGGGSGGGGSDIQMTQSPASLSASVGETVTITCR | |
| | ASGNIHNYLAWYQQKQGRSPQLLVYNAKTLPYGVPSRFSGSGSGTQYSLKINSL | |
| | QPEDFGSYYCQHFWTTPWTFGGGTKLEIK | |
| 15K2- | QVQLQQPGAELVQPGASVKLSCKASGYTFTSYWMNWMKQRPGRGLEWIGRIHP | 245 |
| H(G₄S)₃L | SDSETHYNQKFRTKATLTVDKSSSTAYIQLSSLTSEDSAVYYCAREDGYYWYFD | |
| | VWGAGTTVTVSSGGGGSGGGGSGGGGSNIVLTQSPASLAVSLGQRATISCRASES | |
| | VDIYGNSFMHWYQQKPGQPPKLLIYLASNLESGVPARFSGSGSRTDFTLTIDPVEA | |
| | DDAATYYCQQNNEDPWTFGGGTKLEIK | |
| 15P17- | QVQLQQPGAELVKPGASVKLSCKASGYTFTNYWMNWVKQRPGRGLEWIGRIHP | 246 |
| H(G₄S)₃L | SDSETHYNQKFKSKATLTVDKSSSTAYIQLSSLTSEDSAVYYCAREDGYYWYFD | |
| | VWGAGTTVTVSSGGGGSGGGGSGGGGSNIVLTQSPASLAVSLGQRATISCRASES | |
| | VDSYGNSFMHWYQQKPGQPPKLLIYLASNLESGVPARFSGSGSRTDFTLTIDPVE | |
| | ADDAATYYCQQNHEDPWTFGGGTKLEIK | |
| 15N21- | EVQLVESGGGLVKPGGSLKLSCAASGFTFSSYAMSWVRQTPEKRLEWVAAINSN | 247 |
| H(G₄S)₃L | GGRNYYPDTVKDRFTISRDNAKNTLYLQMSSLRSEDTALYYCARHRGGYYYAM | |
| | DYWGQGTSVTVSSGGGGSGGGGSGGGGSDIVMSQSPSSLAVSVGEKVTMSCKSS | |
| | QSLLYSSNQKNYLAWYQQKPGQSPKLLIYWASTRESGVPDRFTGSGSGTDFTLTI | |
| | SSVKAEDLAVYYCQQYYTYLTFGAGTKLELK | |
| 15K2- | EVQLVQSGAEVKKPGESLKISCKASGYTFTSYWMNWMRQMPGKGLEWIGRIHP | 265 |
| H5(G4S)₃L5 | SDSETHYNPSFQGQATLSVDKSINTAYLQWSSLKASDTAMYYCAREDGYYWYF | |
| | DVWGQGTLVTVSSGGGGSGGGGSGGGGSDIQLTQSPSTLSASVGDRVTITCRASE | |
| | SVDIYGNSFMHWYQQKPGKVPKLLIYLASNLESGVPSRFSGSGSRTEFTLTISSLQ | |
| | PDDFATYYCQQNNEDPWTFGPGTKVDIK | |
| 15K2- | EVQLVESGGGLVQPGRSLRLSCKASGYTFTSYWMNWMRQAPGKGLEWIGRIHP | 266 |
| H4(G4S)₃L5 | SDSETHYNDSVKGRATLSVDKAKNSAYLQMNSLRAEDMALYYCAREDGYYWY | |
| | FDVWGQGTMVTVSSGGGGSGGGGSGGGGSDIQLTQSPSTLSASVGDRVTITCRA | |
| | SESVDIYGNSFMHWYQQKPGKVPKLLIYLASNLESGVPSRFSGSGSRTEFTLTISSL | |
| | QPDDFATYYCQQNNEDPWTFGPGTKVDIK | |
| 3C16- | QVQLVQSGAEVKKPGASVKVSCKASGYTFTSYVLHWVRQAPGQGLEWIGWVIP | 267 |
| H2(G4S)₃L3 | YNDGTKYNEKFKGRATLTSDKSTSTAYMELSSLRSEDTAVYYCARPSNWDEFDY | |
| | WGQGTLVTVSSGGGGSGGGGSGGGGSEIVMTQSPVTVSVSRGGTATLSCRASQN | |
| | VRTAVAWYQQKPGQTPRALIYLASTRASGVPERFSGSGFGTDFTLSISGLQPEDV | |
| | AIYFCLQHWNYPLTFGQGTKVEIK | |
| 15N21- | EVQLLESGGGLVQPGGSLRLSCAASGFTFSSYAMSWVRQAPGKGLEWVAAINSN | 268 |
| H1(G4S)₃L1 | GGRNYYADSVKGRFTISRDNSKNTLYLQMNSLRAEDTAVYYCARHRGGYYYA | |
| | MDYWGQGTLVTVSSGGGGSGGGGSGGGGSDIVMTQSPDSLAVSLGERATINCKS | |
| | SQSLLYSSNQKNYLAWYQQKPGQPPKLLIYWASTRESGVPDRFSGSGSGTDFTLT | |
| | ISSLQAEDVAVYYCQQYYTYLTFGQGTKVEIK | |
| 15N21- | EVQLLESGGGLVQPGGSLRLSCAASGFTFSSYAMSWVRQAPGKGLEWVAAINSN | 269 |
| H1(G4S)₃L1 | GGRNYYADSVKGRFTISRDNSKNTLYLQMNSLRAEDTAVYYCARHRGGYYYA | |
| | MDYWGQGTLVTVSSGGGGSGGGGSGGGGSGGGGSDIVMTQSPDSLAVSLGERA | |
| | TINCKSSQSLLYSSNQKNYLAWYQQKPGQPPKLLIYWASTRESGVPDRFSGSGSG | |
| | TDFTLTISSLQAEDVAVYYCQQYYTYLTFGQGTKVEIK | |
| 15N21- | EVQLVESGGGLVQPGGSLRLSCVASGFTFSSYAMSWVRQAPGKGLEWVASINSN | 270 |
| H3(G4S)₃L1 | GGRNYYSDSVKGRFTISRDNSKNTLYLQMNSLRAEDTAVYYCARHRGGYYYAM | |
| | DYWGQGTLVTVSSGGGGSGGGGSGGGGSDIVMTQSPDSLAVSLGERATINCKSS | |
| | QSLLYSSNQKNYLAWYQQKPGQPPKLLIYWASTRESGVPDRFSGSGSGTDFTLTI | |
| | SSLQAEDVAVYYCQQYYTYLTFGQGTKVEIK | |
| 5A16- | QVQLVQSGAEVKKPGSSVKVSCKASGYTFTRYILHWVRLAPGQGLEWIGYINPY | 271 |
| H1(G4S)₃L1 | NDGTKYNEKFKGKATLTSDKSTNTAYMELSSLRSEDTAVYYCARDSSGYGGAY | |
| | AMDFWGQGTLVTVSSGGGGSGGGGSGGGGSDIQMTQSPSTLSASVGDRVTITCR | |
| | ASGNIHNYLAWYQQKPGKAPKLLVYNAKTLPYGVPARFSGSGSGTEYTLTISSLQ | |
| | PDDFATYYCQHFWFIVWTFGQGTKVEVK | |
| 5A16- | QVQLVQSGAEVKKPGSSVKVSCKASGYTFTRYILHWVRLAPGQGLEWIGYINPY | 272 |
| H1(G4S)₄L1 | NDGTKYNEKFKGKATLTSDKSTNTAYMELSSLRSEDTAVYYCARDSSGYGGAY | |
| | AMDFWGQGTLVTVSSGGGGSGGGGSGGGGSGGGGSDIQMTQSPSTLSASVGDR | |
| | VTITCRASGNIHNYLAWYQQKPGKAPKLLVYNAKTLPYGVPARFSGSGSGTEYT | |
| | LTISSLQPDDFATYYCQHFWTTPWTFGQGTKVEVK | |
| 5A16- | QVQLVQSGAEVKKPGASVKVSCKASGYTFTRYILHWVRLAPGQGLEWIGYINPY | 273 |
| H2(G4S)₃L3 | NDGTKYNQKFKGKATLTSDKSTNTAYMELSSLRSEDTAVYYCARDSSGYGGAY | |
| | AMDFWGQGTLVTVSSGGGGSGGGGSGGGGSDIQMTQSPSSLSASVGDRVTITCK | |
| | ASGNIHNYLAWYQQKPGKAPKLLVYNAKYRYSGVPSRFSGSGSGTDYTLTISSL | |
| | QPEDFATYYCQHFWFIVWTFGQGTKVEIK | |
| 5A16- | QVQLVQSGAEVKKPGASVKVSCKASGYTFTRYILHWVRLAPGQGLEWIGWINPY | 274 |
| H4(G4S)₃L3 | NDGTQYNEKFKGRATLTSDTSTSTAYMELSSLRSEDTAVYYCARDSSGYGGAYA | |
| | MDFWGQGTTVTVSSGGGGSGGGGSGGGGSDIQMTQSPSSLSASVGDRVTITCKA | |
| | SGNIHNYLAWYQQKPGKAPKLLVYNAKYRYSGVPSRFSGSGSGTDYTLTISSLQP | |
| | EDFATYYCQHFWTTPWTFGQGTKVEIK | |
| 5A16- | QVQLVQSGAEVKKPGASVKVSCKASGYTFTRYILHWVRLAPGQGLEWIGWINPY | 275 |
| H4(G4S)₄L3 | NDGTQYNEKFKGRATLTSDTSTSTAYMELSSLRSEDTAVYYCARDSSGYGGAYA | |
| | MDFWGQGTTVTVSSGGGGSGGGGSGGGGSGGGGSDIQMTQSPSSLSASVGDRV | |
| | TITCKASGNIHNYLAWYQQKPGKAPKLLVYNAKYRYSGVPSRFSGSGSGTDYTL | |
| | TISSLQPEDFATYYCQHFWTTPWTFGQGTKVEIK | |
| 8H5- | EVTLKESGPVLVKPTETLTLTCSFSGFSLSTFGMGVGWIRQPPGKALEWLAHIWW | 276 |
| H5(G4S)₃L5 | DDDKYYNPALKSRLTISKDTSKSQVVLTMTNMDPVDTATYYCARTYDYDEYFD | |
| | YWGQGTLVTVSSGGGGSGGGGSGGGGSDIVMTQSPDSLAVSLGERATINCRSSK | |
| | SLLHSNGITYFYWYQQKPGQPPKLLIYQMSNLASGVPDRFSSSGSGTDFTLTISSL | |
| | QAEDVAVYYCAQNLELPFTFGQGTKVEIK | |
| 8H5- | EVTLKESGPVLVKPTETLTLTCSFSGFSLSTFGMGVGWIRQPPGKALEWLAHIWW | 277 |
| H5(G4S)₃L6 | DDDKYYNPALKSRLTISKDTSKSQVVLTMTNMDPVDTATYYCARTYDYDEYFD | |
| | YWGQGTLVTVSSGGGGSGGGGSGGGGSEIVMTQSPATLSLSPGERATLSCRSSKS | |
| | LLHSNGITYFYWYQQKPGQAPRLLIYQMSNLASGIPARFSSSGSGTDFTLTISSLEP | |
| | EDFAVYYCAQNLELPFTFGQGTKLEIK | |
| 3121- | EVQLLESGGGLVQPGGSLRLSCKASGYTFTNYWMNWVRQAPGKGLEWIGRIHPS | 278 |
| H7(G4S)₃L3 | DSETHYNDSVKGRATLSVDKSKNTAYLQMNSLRAEDTAVYYCARYDGYFAYW | |
| | GQGTLVTVSSGGGGSGGGGSGGGGSDIVMTQSPDSLAVSLGERATINCRASDHIN | |
| | NWLAWYQQKPGQPPKLLISGATSLESGVPDRFSGSGSGKDYTLTISSLQAEDVAV | |
| | YYCQQYWSIPFTFGQGTKVEIK | |
| 10P18- | QVQLVQSGAEVKKPGSSVKVSCKASGYSFTGYYIDWVRQAPGQGLEWIGYIYPS | 279 |
| H1(G4S)₃L2 | NGETSYNQKFKGRATLTVDKSTSTVYMELSSLRSEDTAVYYCARESYAMDYWG | |
| | QGTLVTVSSGGGGSGGGGSGGGGSDIVLTQSPDSLAVSLGERATINCRASKSVST | |
| | SGYSYLAWYQQKPGQPPKLLIYLASNLESGVPDRFSGSGSGTDFTLTISSLQAEDV | |
| | AVYYCQHSRELPYTFGGGTKVEIK | |
| 10P18- | QVQLVQSGAEVKKPGASVKVSCKASGYSFTGYYIDWVRQAPGQGLEWIGYIYPS | 280 |
| H2(G4S)₃L2 | NGETSYNQKFKGRATLTVDTSTSTVYMELSSLRSEDTAVYYCARESYAMDYWG | |
| | QGTTVTVSSGGGGSGGGGSGGGGSDIVLTQSPDSLAVSLGERATINCRASKSVST | |
| | SGYSYLAWYQQKPGQPPKLLIYLASNLESGVPDRFSGSGSGTDFTLTISSLQAEDV | |
| | AVYYCQHSRELPYTFGGGTKVEIK | |
| 5A24- | QVQLVQSGAEVKKPGASVKVSCKASGYKFTDFNMDWVRQAPGQGLEWIGDINP | 281 |
| H5(G4S)₃L5 | NSGGTIYNEKFKNRATLTVDKSISTAYMELSRLRSDDTAVYYCARWDYGNFAY | |
| | WGQGTLVTVSSGGGGSGGGGSGGGGSDIVMTQSPLSLPVTPGEPASISCRSSKSL | |
| | LHSNGITYLYWYLQKPGQSPQLLIYQMSNLASGVPDRFSSSGSGTDFTLKISRVEA | |
| | EDVGVYYCAQNLELPLTFGQGTKVEIK | |
| 5A24- | QVQLVQSGAEVKKPGASVKVSCKASGYKFTDFNMDWVRQAPGQGLEWIGDINP | 282 |
| H5(G4S)₃L7 | NSGGTIYNEKFKNRATLTVDKSISTAYMELSRLRSDDTAVYYCARWDYGNFAY | |
| | WGQGTLVTVSSGGGGSGGGGSGGGGSDIQMTQSPSTLSASVGDRVTITCRSSKSL | |
| | LHSNGITYLYWYQQKPGKAPKLLIYQMSNLASGVPARFSSSGSGTEFTLTISSLQP | |
| | DDFATYYCAQNLELPLTFGQGTKVEVK | |
| 15P17- | EVTLKESGPTLVKPTQTLTLTCTASGYTFTNYWMNWVRQPPGKALEWIGRIHPS | 283 |
| H4(G4S)₃L2 | DSETHYNQKFKSRATLTVDTSKNQAVLTMTNMDPVDTATYYCAREDGYYWYF | |
| | DVWGQGTLVTVSSGGGGSGGGGSGGGGSDIVLTQSPDSLAVSLGERATINCRAS | |
| | ESVDSYGNSFMHWYQQKPGQPPKLLIYLASNLESGVPDRFSGSGSRTDFTLTISSL | |
| | QAEDVAVYYCQQNHEDPWTFGQGTKVEIK | |
| 15P17- | EVTLKESGPTLVKPTQTLTLTCTASGYTFWVRQPPGKALEWIGRIHPS | 284 |
| H3(G4S)₃L1 | DSETHYNQKFKSRATLTVDKSKNQAVLTMTNMDPVDTATYYCAREDGYYWYF | |
| | DVWGQGTLVTVSSGGGGSGGGGSGGGGSDIQLTQSPSSVSASVGDRVTITCRASE | |
| | SVDSYGNSFLHWYQQKPGKAPKLLIYLASSLQSGVPSRFSGS | |
| 14L22- | EVQLLESGGGLVQPGGSLRLSCAASGFTFSSYAMSWVRQAPGKGLEWVAAINSN | 285 |
| H1(G4S)₃L1 | GGNTYYADSVKGRFTISRDNSKNTLYLQMNSLRAEDTAVYYCARHRGGFYYAV | |
| | DYWGQGTLVTVSSGGGGSGGGGSGGGGSDIVMTQSPDSLAVSLGERATINCKSS | |
| | QSVLYSSNQKNYLAWYQQKPGQPPKLLIYWASTRESGVPDRFSGSGSGTDFTLTI | |
| | SSLQAEDVAVYYCHQYLSSRTFGQGTRLEIK | |
| 14L22- | QVELVESGGGLVQPGGSLRLSCAASGFTFSSYAMSWVRQAPGKGLEWVAAINSN | 286 |
| H2(G4S)₃L1 | GGNTYYADSVKGRFTISRDNSKNTLYLQMNSLRAEDTAVYYCARHRGGFYYAV | |
| | DYWGQGTLVTVSSGGGGSGGGGSGGGGSDIVMTQSPDSLAVSLGERATINCKSS | |
| | QSVLYSSNQKNYLAWYQQKPGQPPKLLIYWASTRESGVPDRFSGSGSGTDFTLTI | |
| | SSLQAEDVAVYYCHQYLSSRTFGQGTRLEIK | |

### Example 4: ELISA Binding Analysis of scFvs

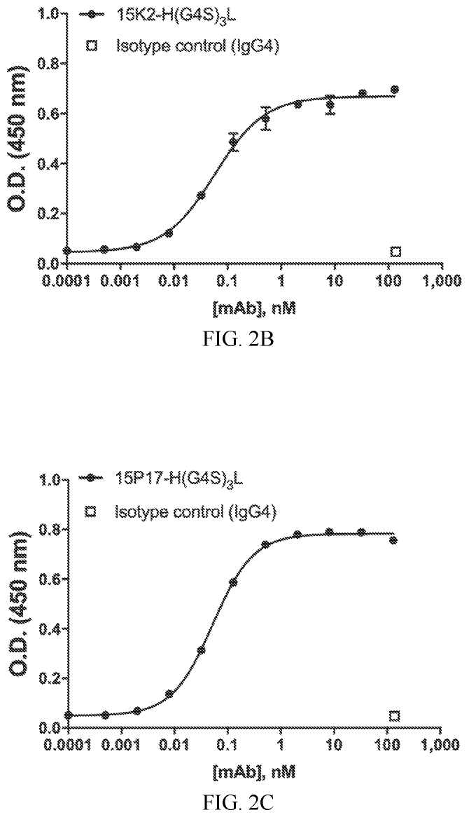

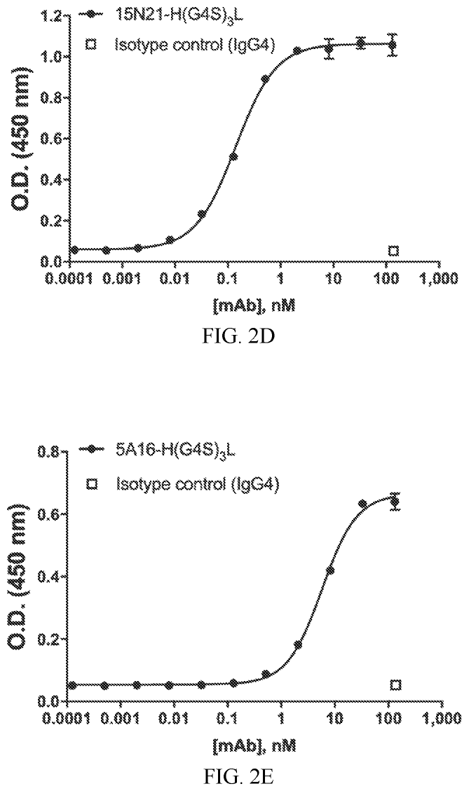

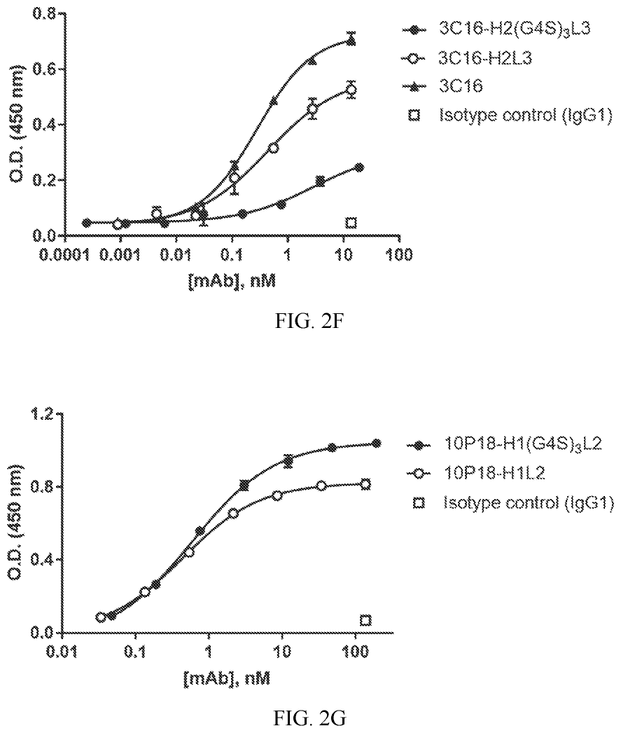

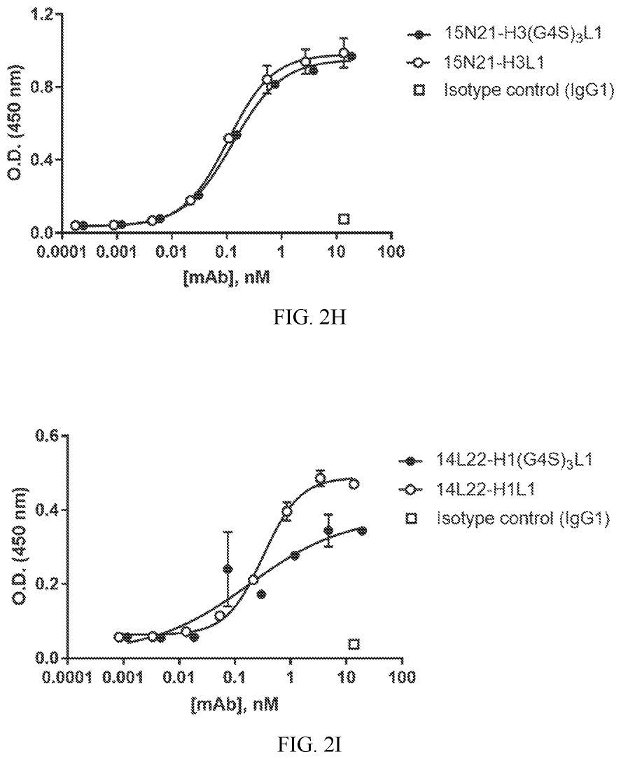

Fusion proteins of scFvs fused to one (G₄S) linker and human IgG4 Fc (with the order of scFv, G₄S linker and Fc from the N-terminus to the C-terminus) were tested for their ability to bind human DLL3 using the ELISA method as described in PCT Application No. PCT/US2019/029888, filed on Apr. 30, 2019, which is incorporated herein by reference in its entirety. The results of the ELISA assay are shown in FIGS. 2A-2I.

### Example 5: FACS Analysis of Humanized scFvs

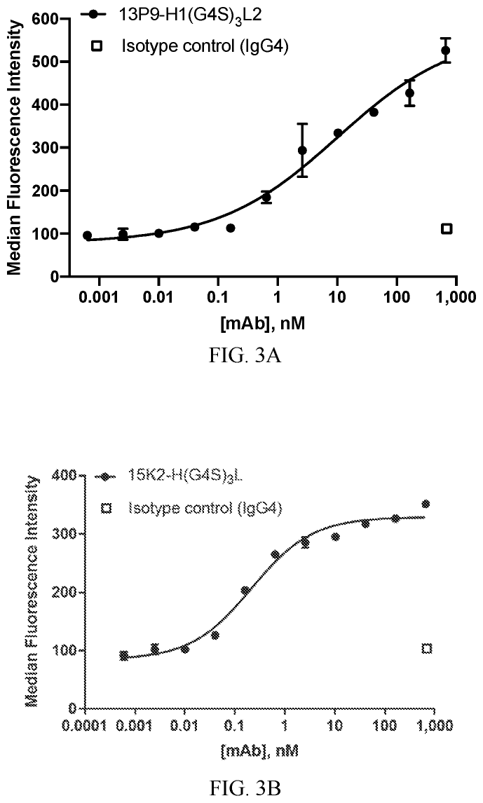

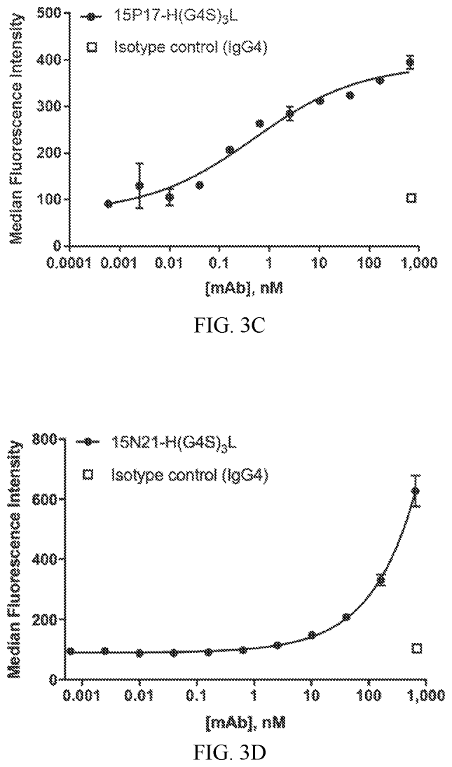

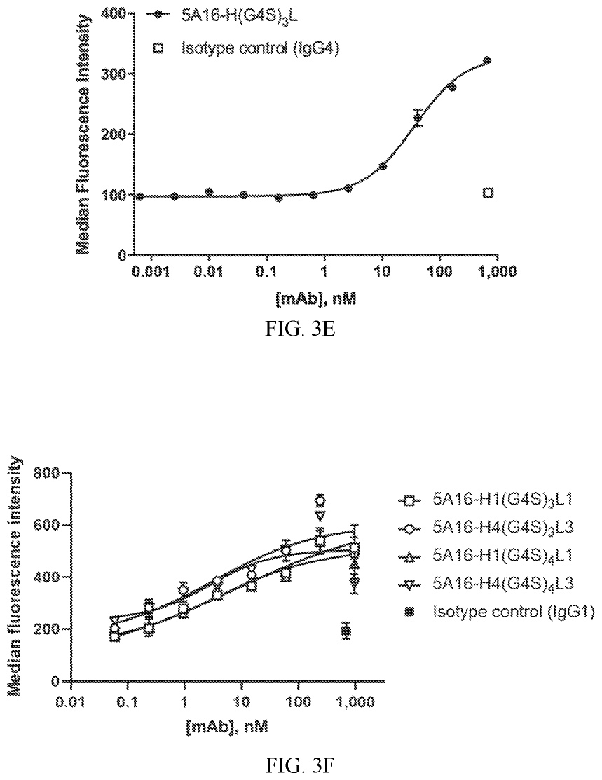

The binding of the scFv and Fc fusion proteins to huDLL3 on the surface of HEK293 cells was measured by FACS using the method described in PCT Application No. PCT/US2019/029888, filed on Apr. 30, 2019, which is incorporated herein by reference in its entirety, with one modification that propidium iodide (PI) (Thermo Fisher Cat#: P3566) was added together with the secondary antibody to label dead cells. The binding results are shown in FIGS. 3A-3F.

### Example 6: Construction of Chimeric Antigen Receptor Constructs Comprising Anti-DLL3 Monoclonal Antibodies or Antigen-Binding Fragments Thereof

To construct a CAR, the mAbs were converted into scFv using the VH, VL and a (G₄S)ₙ linker, and the scFv was fused to the N-terminus of the hinge and transmembrane domains derived from human CD8α (aa 114-188, Boursier J P et al., J Biol Chem. 1993; 268(3): 2013-20). The C-terminal intracellular signaling domain of the CAR was constructed by fusing the intracellular costimulatory domain of CD28 (aa 162-202, Aruffo A and Seed B, Proc Natl Acad Sci USA. 1987; 84(23):8573-7) followed by the activation domain from CD3 zeta chain (aa 52-162, Letourneur F and Klausner R D, Proc Natl Acad Sci USA. 1991; 88(20):8905-9). The DNA sequence encoding the CAR was assembled and cloned into an expression vector (either retroviral, lentiviral, extrachromosomal or integrated) to generate the CAR construct using standard molecular biology cloning techniques.

### Example 7: Tumor Cell Killing Assay to Assess the Activity of CAR T Cells

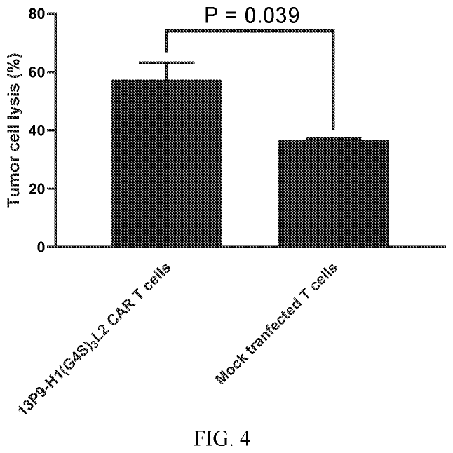

CD4+/CD8+ T cells were isolated using the Pan T isolation kit (Miltenyi biotech, Cat#: 130-096-535), and activated for 3 days by Dynabeads™ Human T-Activator CD3/CD28 (ThermoFisher, Cat#: 11131D) in AIM V medium (ThermoFisher, Cat#: 12055083) containing 10% FBS according to the manufacture instructions. Next, active T cells were continuously cultured for less than a week in AIM V medium containing 10% FBS and 300 IU/ml IL2 (R&D systems, Cat#: 202-IL-050) and transiently transfected with the 13P9-H1(G4S)₃L2 CAR expression plasmid by electroporation to obtain the CAR T cells. Active T cells were also mock transfected and used as a negative control. Following a 48-hour recovery period, the CAR T cells and active T cells were used in the assay as the effector cells. Target cells HEK293-DLL3 were stained with CFSE (ThermoFisher, Cat#: C34554) and co-cultured with the CAR T cells or active T cells for 24 hours at the E/T (effector/target) ratio of 5:1. Next, the cells were stained with PI (ThermoFisher, Cat#: P3566) and Annexin V (Biolegend, Cat#: 640924) and analyzed by flow cytometry (Attune NxT). Only CFSE positive cells were counted. The tumor cell lysis percentages were calculated as the percentage of PI and/or Annexin V positive cells and shown in FIG. 4.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

1. An isolated polynucleotide comprising a nucleic acid sequence encoding a chimeric antigen receptor (CAR), wherein the CAR comprises:
(a) an extracellular domain comprising at least one antigen binding domain that specifically binds DLL3;
(b) a hinge region;
(c) a transmembrane region; and
(d) an intracellular signaling domain.

2. The isolated polynucleotide of claim 1, wherein the antigen binding domain comprises a heavy chain complementarity determining region 1 (HCDR1), HCDR2, HCDR3, a light chain complementarity determining region 1 (LCDR1), LCDR2, and LCDR3, having the polypeptide sequences of:
(1) SEQ ID NOs: 25, 26, 27, 61, 62 and 63, respectively, or SEQ ID NOs: 97, 98, 99, 133, 134 and 135, respectively;
(2) SEQ ID NOs: 28, 29, 30, 64, 65 and 66, respectively, or SEQ ID NOs: 100, 101, 102, 136, 137 and 138, respectively;
(3) SEQ ID NOs: 31, 32, 33, 67, 68 and 69, respectively, or SEQ ID NOs: 103, 104, 105, 139, 140 and 141, respectively;
(4) SEQ ID NOs: 34, 35, 36, 70, 71 and 72, respectively, or SEQ ID NOs: 106, 107, 108, 142, 143 and 144, respectively;
(5) SEQ ID NOs: 37, 38, 39, 73, 74 and 75, respectively, or SEQ ID NOs: 109, 110, 111, 145, 146 and 147, respectively;
(6) SEQ ID NOs: 40, 41, 42, 76, 77 and 78, respectively, or SEQ ID NOs: 112, 113, 114, 148, 149 and 150, respectively;
(7) SEQ ID NOs: 43, 44, 45, 79, 80 and 81, respectively, or SEQ ID NOs: 115, 116, 117, 151, 152 and 153, respectively;
(8) SEQ ID NOs: 46, 47, 48, 82, 83 and 84, respectively, or SEQ ID NOs: 118, 119, 120, 154, 155 and 156, respectively;
(9) SEQ ID NOs: 49, 50, 51, 85, 86 and 87, respectively, or SEQ ID NOs: 121, 122, 123, 157, 158 and 159, respectively;
(10) SEQ ID NOs: 52, 53, 54, 88, 89 and 90, respectively, or SEQ ID NOs: 124, 125, 126, 160, 161 and 162, respectively;
(11) SEQ ID NOs: 55, 56, 57, 91, 92 and 93, respectively, or SEQ ID NOs: 127, 128, 129, 163, 164 and 165, respectively; or
(12) SEQ ID NOs: 58, 59, 60, 94, 95 and 96, respectively, or SEQ ID NOs: 130, 131, 132, 166, 167 and 168, respectively.

3. (canceled)

4. The isolated polynucleotide of claim 1, wherein the antigen binding domain comprises a heavy chain variable region having a polypeptide sequence at least 95% identical to SEQ ID NO: 1, 3, 5, 7, 9, 11, 13, 15, 17, 19, 21, 23, 170, 175-209, or 248-255, or a light chain variable region having a polypeptide sequence at least 95% identical to SEQ ID NO: 2, 4, 6, 8, 10, 12, 14, 16, 18, 20, 22, 24, 171-174, 210-240, or 256-264.

5. The isolated polynucleotide of claim 1, wherein the antigen binding domain comprises:
(1) a heavy chain variable region having the polypeptide sequence of SEQ ID NO:1, and a light chain variable region having the polypeptide sequence of SEQ ID NO:2;
(2) a heavy chain variable region having the polypeptide sequence of SEQ ID NO:3, and a light chain variable region having the polypeptide sequence of SEQ ID NO:4;
(3) a heavy chain variable region having the polypeptide sequence of SEQ ID NO:5, and a light chain variable region having the polypeptide sequence of SEQ ID NO:6;
(4) a heavy chain variable region having the polypeptide sequence of SEQ ID NO:7, and a light chain variable region having the polypeptide sequence of SEQ ID NO:8;
(5) a heavy chain variable region having the polypeptide sequence of SEQ ID NO:9, and a light chain variable region having the polypeptide sequence of SEQ ID NO:10;
(6) a heavy chain variable region having the polypeptide sequence of SEQ ID NO:11, and a light chain variable region having the polypeptide sequence of SEQ ID NO:12;
(7) a heavy chain variable region having the polypeptide sequence of SEQ ID NO:13, and a light chain variable region having the polypeptide sequence of SEQ ID NO:14;
(8) a heavy chain variable region having the polypeptide sequence of SEQ ID NO:15, and a light chain variable region having the polypeptide sequence of SEQ ID NO:16;
(9) a heavy chain variable region having the polypeptide sequence of SEQ ID NO:17, and a light chain variable region having the polypeptide sequence of SEQ ID NO:18;
(10) a heavy chain variable region having the polypeptide sequence of SEQ ID NO:19, and a light chain variable region having the polypeptide sequence of SEQ ID NO:20;
(11) a heavy chain variable region having the polypeptide sequence of SEQ ID NO:21, and a light chain variable region having the polypeptide sequence of SEQ ID NO:22;
(12) a heavy chain variable region having the polypeptide sequence of SEQ ID NO:23, and a light chain variable region having the polypeptide sequence of SEQ ID NO:24;
(13) a heavy chain variable region having the polypeptide sequence of SEQ ID NO:170, and a light chain variable region having the polypeptide sequence of SEQ ID NO:171;
(14) a heavy chain variable region having the polypeptide sequence of SEQ ID NO:170, and a light chain variable region having the polypeptide sequence of SEQ ID NO:172;
(15) a heavy chain variable region having the polypeptide sequence of SEQ ID NO:170, and a light chain variable region having the polypeptide sequence of SEQ ID NO:173.
(16) a heavy chain variable region having the polypeptide sequence of SEQ ID NO:183, and a light chain variable region having the polypeptide sequence of SEQ ID NO:217;
(17) a heavy chain variable region having the polypeptide sequence of SEQ ID NO:183, and a light chain variable region having the polypeptide sequence of SEQ ID NO:218;
(18) a heavy chain variable region having the polypeptide sequence of SEQ ID NO:184, and a light chain variable region having the polypeptide sequence of SEQ ID NO:217;
(19) a heavy chain variable region having the polypeptide sequence of SEQ ID NO:184, and a light chain variable region having the polypeptide sequence of SEQ ID NO:218;
(20) a heavy chain variable region having the polypeptide sequence of SEQ ID NO:198, and a light chain variable region having the polypeptide sequence of SEQ ID NO:229;
(21) a heavy chain variable region having the polypeptide sequence of SEQ ID NO:200, and a light chain variable region having the polypeptide sequence of SEQ ID NO:229;
(22) a heavy chain variable region having the polypeptide sequence of SEQ ID NO:198, and a light chain variable region having the polypeptide sequence of SEQ ID NO:231;
(23) a heavy chain variable region having the polypeptide sequence of SEQ ID NO:200, and a light chain variable region having the polypeptide sequence of SEQ ID NO:231;
(24) a heavy chain variable region having the polypeptide sequence of SEQ ID NO:201, and a light chain variable region having the polypeptide sequence of SEQ ID NO:229;
(25) a heavy chain variable region having the polypeptide sequence of SEQ ID NO:201, and a light chain variable region having the polypeptide sequence of SEQ ID NO:230;
(26) a heavy chain variable region having the polypeptide sequence of SEQ ID NO:201, and a light chain variable region having the polypeptide sequence of SEQ ID NO:231;
(27) a heavy chain variable region having the polypeptide sequence of SEQ ID NO:175, and a light chain variable region having the polypeptide sequence of SEQ ID NO:210;
(28) a heavy chain variable region having the polypeptide sequence of SEQ ID NO:175, and a light chain variable region having the polypeptide sequence of SEQ ID NO:211;
(29) a heavy chain variable region having the polypeptide sequence of SEQ ID NO:175, and a light chain variable region having the polypeptide sequence of SEQ ID NO:212;
(30) a heavy chain variable region having the polypeptide sequence of SEQ ID NO:176, and a light chain variable region having the polypeptide sequence of SEQ ID NO:210;
(31) a heavy chain variable region having the polypeptide sequence of SEQ ID NO:176, and a light chain variable region having the polypeptide sequence of SEQ ID NO:211;
(32) a heavy chain variable region having the polypeptide sequence of SEQ ID NO:176, and a light chain variable region having the polypeptide sequence of SEQ ID NO:212;
(33) a heavy chain variable region having the polypeptide sequence of SEQ ID NO:177, and a light chain variable region having the polypeptide sequence of SEQ ID NO:210;
(34) a heavy chain variable region having the polypeptide sequence of SEQ ID NO:177, and a light chain variable region having the polypeptide sequence of SEQ ID NO:211;
(35) a heavy chain variable region having the polypeptide sequence of SEQ ID NO:178, and a light chain variable region having the polypeptide sequence of SEQ ID NO:210;
(36) a heavy chain variable region having the polypeptide sequence of SEQ ID NO:178, and a light chain variable region having the polypeptide sequence of SEQ ID NO:211;
(37) a heavy chain variable region having the polypeptide sequence of SEQ ID NO:177, and a light chain variable region having the polypeptide sequence of SEQ ID NO:211;
(38) a heavy chain variable region having the polypeptide sequence of SEQ ID NO:177, and a light chain variable region having the polypeptide sequence of SEQ ID NO:212;
(39) a heavy chain variable region having the polypeptide sequence of SEQ ID NO:178, and a light chain variable region having the polypeptide sequence of SEQ ID NO:212;
(40) a heavy chain variable region having the polypeptide sequence of SEQ ID NO:179, and a light chain variable region having the polypeptide sequence of SEQ ID NO:213;
(41) a heavy chain variable region having the polypeptide sequence of SEQ ID NO:179, and a light chain variable region having the polypeptide sequence of SEQ ID NO:214;
(42) a heavy chain variable region having the polypeptide sequence of SEQ ID NO:179, and a light chain variable region having the polypeptide sequence of SEQ ID NO:215;
(43) a heavy chain variable region having the polypeptide sequence of SEQ ID NO:180, and a light chain variable region having the polypeptide sequence of SEQ ID NO:213;
(44) a heavy chain variable region having the polypeptide sequence of SEQ ID NO:180, and a light chain variable region having the polypeptide sequence of SEQ ID NO:214;
(45) a heavy chain variable region having the polypeptide sequence of SEQ ID NO:180, and a light chain variable region having the polypeptide sequence of SEQ ID NO:215;
(46) a heavy chain variable region having the polypeptide sequence of SEQ ID NO:181, and a light chain variable region having the polypeptide sequence of SEQ ID NO:213;
(47) a heavy chain variable region having the polypeptide sequence of SEQ ID NO:181, and a light chain variable region having the polypeptide sequence of SEQ ID NO:214;
(48) a heavy chain variable region having the polypeptide sequence of SEQ ID NO:182, and a light chain variable region having the polypeptide sequence of SEQ ID NO:215;
(49) a heavy chain variable region having the polypeptide sequence of SEQ ID NO:202, and a light chain variable region having the polypeptide sequence of SEQ ID NO:232;
(50) a heavy chain variable region having the polypeptide sequence of SEQ ID NO:202, and a light chain variable region having the polypeptide sequence of SEQ ID NO:233;
(51) a heavy chain variable region having the polypeptide sequence of SEQ ID NO:202, and a light chain variable region having the polypeptide sequence of SEQ ID NO:234;
(52) a heavy chain variable region having the polypeptide sequence of SEQ ID NO:203, and a light chain variable region having the polypeptide sequence of SEQ ID NO:232;
(53) a heavy chain variable region having the polypeptide sequence of SEQ ID NO:203, and a light chain variable region having the polypeptide sequence of SEQ ID NO:233;
(54) a heavy chain variable region having the polypeptide sequence of SEQ ID NO:203, and a light chain variable region having the polypeptide sequence of SEQ ID NO:234;
(55) a heavy chain variable region having the polypeptide sequence of SEQ ID NO:204, and a light chain variable region having the polypeptide sequence of SEQ ID NO:234;
(56) a heavy chain variable region having the polypeptide sequence of SEQ ID NO:208, and a light chain variable region having the polypeptide sequence of SEQ ID NO:239;
(57) a heavy chain variable region having the polypeptide sequence of SEQ ID NO:208, and a light chain variable region having the polypeptide sequence of SEQ ID NO:240;
(58) a heavy chain variable region having the polypeptide sequence of SEQ ID NO:253, and a light chain variable region having the polypeptide sequence of SEQ ID NO:261; or
(59) a heavy chain variable region having the polypeptide sequence of SEQ ID NO:255, and a light chain variable region having the polypeptide sequence of SEQ ID NO:263.

6-7. (canceled)

8. The isolated polynucleotide of claim 1, wherein the antigen binding domain is a single chain variable fragment (scFv).

9. The isolated polynucleotide of claim 8, wherein the single chain variable fragment (scFv) is humanized.

10. The isolated polynucleotide of claim 8, wherein the single chain variable fragment (scFv) comprises a polypeptide sequence at least 95% identical to any one of SEQ ID NOs: 241-247 or 265-286.

11. The isolated polynucleotide of claim 1, wherein the chimeric antigen receptor (CAR) comprises one or more antigen binding domains, and/or wherein the intracellular signaling domain comprises one or more costimulatory domains and one or more activating domains.

12. (canceled)

13. A chimeric antigen receptor (CAR) encoded by the isolated polynucleotide of claim 1.

14. A vector comprising the isolated polynucleotide of claim 1.

15. A host cell comprising the vector of claim 14.

16. The host cell of claim 15, wherein the host cell is a T cell or a NK cell.

17. (canceled)

18. A method of making a host cell expressing a chimeric antigen receptor (CAR), the method comprising transducing a T cell or a NK cell with the vector of claim 14.

19. A method of producing a chimeric antigen receptor (CAR)-T cell or a chimeric antigen receptor (CAR)-NK cell, the method comprising culturing T cells or NK cells comprising the isolated polynucleotide comprising a nucleic acid encoding a chimeric antigen receptor (CAR) of claim 1 under conditions to produce the CAR-T cell or CAR-NK cell and recovering the CAR-T cell or CAR-NK cell.

20-21. (canceled)

22. A method of generating a cell comprising a chimeric antigen receptor (CAR), the method comprising contacting a cell with the isolated polynucleotide comprising a nucleic acid encoding a chimeric antigen receptor (CAR) of claim 1, wherein the isolated polynucleotide is an in vitro transcribed RNA or synthetic RNA.

23. A method of treating cancer in a subject in need thereof, comprise administering to the subject the host cell of claim 15.

24. The method of claim 23, wherein the cancer is selected from a lung cancer such as small cell lung cancer (SCLC), large cell neuroendocrine carcinoma (LCNEC), a gastric cancer, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, and other solid tumors, and a non-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

25. The method of claim 23, further comprising administering to the subject in need thereof an agent that increases the efficacy of a cell expressing a CAR, an agent that ameliorates one or more side effects associated with administration of a cell expressing a CAR molecule, or an agent that treats the disease associated with DLL3.

26-27. (canceled)

28. A humanized anti-DLL3 monoclonal antibody or antigen-binding fragment thereof, wherein the antibody or antigen-binding fragment thereof comprises a heavy chain variable region having a polypeptide sequence at least 95% identical to any one of SEQ ID NOs: 170, 175-209 or 248-255, or a light chain variable region having a polypeptide sequence at least 95% identical to any one of SEQ ID NOs: 171-174, 210-240 or 256-264.

29. The humanized anti-DLL3 monoclonal antibody or antigen-binding fragment thereof of claim 28, wherein the antibody or antigen-binding fragment thereof comprises:
(1) a heavy chain variable region having the polypeptide sequence of SEQ ID NO:170, and a light chain variable region having the polypeptide sequence of SEQ ID NO:171;
(2) a heavy chain variable region having the polypeptide sequence of SEQ ID NO:170, and a light chain variable region having the polypeptide sequence of SEQ ID NO:172;
(3) a heavy chain variable region having the polypeptide sequence of SEQ ID NO:170, and a light chain variable region having the polypeptide sequence of SEQ ID NO:173.
(4) a heavy chain variable region having the polypeptide sequence of SEQ ID NO:183, and a light chain variable region having the polypeptide sequence of SEQ ID NO:217;
(5) a heavy chain variable region having the polypeptide sequence of SEQ ID NO:183, and a light chain variable region having the polypeptide sequence of SEQ ID NO:218;
(6) a heavy chain variable region having the polypeptide sequence of SEQ ID NO:184, and a light chain variable region having the polypeptide sequence of SEQ ID NO:217;
(7) a heavy chain variable region having the polypeptide sequence of SEQ ID NO:184, and a light chain variable region having the polypeptide sequence of SEQ ID NO:218;
(8) a heavy chain variable region having the polypeptide sequence of SEQ ID NO:198, and a light chain variable region having the polypeptide sequence of SEQ ID NO:229;
(9) a heavy chain variable region having the polypeptide sequence of SEQ ID NO:200, and a light chain variable region having the polypeptide sequence of SEQ ID NO:229;
(10) a heavy chain variable region having the polypeptide sequence of SEQ ID NO:198, and a light chain variable region having the polypeptide sequence of SEQ ID NO:231;
(11) a heavy chain variable region having the polypeptide sequence of SEQ ID NO:200, and a light chain variable region having the polypeptide sequence of SEQ ID NO:231;
(12) a heavy chain variable region having the polypeptide sequence of SEQ ID NO:201, and a light chain variable region having the polypeptide sequence of SEQ ID NO:229;
(13) a heavy chain variable region having the polypeptide sequence of SEQ ID NO:201, and a light chain variable region having the polypeptide sequence of SEQ ID NO:230;
(14) a heavy chain variable region having the polypeptide sequence of SEQ ID NO:201, and a light chain variable region having the polypeptide sequence of SEQ ID NO:231;
(15) a heavy chain variable region having the polypeptide sequence of SEQ ID NO:175, and a light chain variable region having the polypeptide sequence of SEQ ID NO:210;
(16) a heavy chain variable region having the polypeptide sequence of SEQ ID NO:175, and a light chain variable region having the polypeptide sequence of SEQ ID NO:211;
(17) a heavy chain variable region having the polypeptide sequence of SEQ ID NO:175, and a light chain variable region having the polypeptide sequence of SEQ ID NO:212;
(18) a heavy chain variable region having the polypeptide sequence of SEQ ID NO:176, and a light chain variable region having the polypeptide sequence of SEQ ID NO:210;
(19) a heavy chain variable region having the polypeptide sequence of SEQ ID NO:176, and a light chain variable region having the polypeptide sequence of SEQ ID NO:211;
(20) a heavy chain variable region having the polypeptide sequence of SEQ ID NO:176, and a light chain variable region having the polypeptide sequence of SEQ ID NO:212;
(21) a heavy chain variable region having the polypeptide sequence of SEQ ID NO:177, and a light chain variable region having the polypeptide sequence of SEQ ID NO:210;
(22) a heavy chain variable region having the polypeptide sequence of SEQ ID NO:177, and a light chain variable region having the polypeptide sequence of SEQ ID NO:211;
(23) a heavy chain variable region having the polypeptide sequence of SEQ ID NO:178, and a light chain variable region having the polypeptide sequence of SEQ ID NO:210;
(24) a heavy chain variable region having the polypeptide sequence of SEQ ID NO:178, and a light chain variable region having the polypeptide sequence of SEQ ID NO:211;
(25) a heavy chain variable region having the polypeptide sequence of SEQ ID NO:177, and a light chain variable region having the polypeptide sequence of SEQ ID NO:211;
(26) a heavy chain variable region having the polypeptide sequence of SEQ ID NO:177, and a light chain variable region having the polypeptide sequence of SEQ ID NO:212;
(27) a heavy chain variable region having the polypeptide sequence of SEQ ID NO:178, and a light chain variable region having the polypeptide sequence of SEQ ID NO:212;
(28) a heavy chain variable region having the polypeptide sequence of SEQ ID NO:179, and a light chain variable region having the polypeptide sequence of SEQ ID NO:213;
(29) a heavy chain variable region having the polypeptide sequence of SEQ ID NO:179, and a light chain variable region having the polypeptide sequence of SEQ ID NO:214;
(30) a heavy chain variable region having the polypeptide sequence of SEQ ID NO:179, and a light chain variable region having the polypeptide sequence of SEQ ID NO:215;
(31) a heavy chain variable region having the polypeptide sequence of SEQ ID NO:180, and a light chain variable region having the polypeptide sequence of SEQ ID NO:213;
(32) a heavy chain variable region having the polypeptide sequence of SEQ ID NO:180, and a light chain variable region having the polypeptide sequence of SEQ ID NO:214;
(33) a heavy chain variable region having the polypeptide sequence of SEQ ID NO:180, and a light chain variable region having the polypeptide sequence of SEQ ID NO:215;
(34) a heavy chain variable region having the polypeptide sequence of SEQ ID NO:181, and a light chain variable region having the polypeptide sequence of SEQ ID NO:213;
(35) a heavy chain variable region having the polypeptide sequence of SEQ ID NO:181, and a light chain variable region having the polypeptide sequence of SEQ ID NO:214;
(36) a heavy chain variable region having the polypeptide sequence of SEQ ID NO:182, and a light chain variable region having the polypeptide sequence of SEQ ID NO:215;
(37) a heavy chain variable region having the polypeptide sequence of SEQ ID NO:202, and a light chain variable region having the polypeptide sequence of SEQ ID NO:232;
(38) a heavy chain variable region having the polypeptide sequence of SEQ ID NO:202, and a light chain variable region having the polypeptide sequence of SEQ ID NO:233;
(39) a heavy chain variable region having the polypeptide sequence of SEQ ID NO:202, and a light chain variable region having the polypeptide sequence of SEQ ID NO:234;
(40) a heavy chain variable region having the polypeptide sequence of SEQ ID NO:203, and a light chain variable region having the polypeptide sequence of SEQ ID NO:232;
(41) a heavy chain variable region having the polypeptide sequence of SEQ ID NO:203, and a light chain variable region having the polypeptide sequence of SEQ ID NO:233;
(42) a heavy chain variable region having the polypeptide sequence of SEQ ID NO:203, and a light chain variable region having the polypeptide sequence of SEQ ID NO:234;
(43) a heavy chain variable region having the polypeptide sequence of SEQ ID NO:204, and a light chain variable region having the polypeptide sequence of SEQ ID NO:234;
(44) a heavy chain variable region having the polypeptide sequence of SEQ ID NO:208, and a light chain variable region having the polypeptide sequence of SEQ ID NO:239;
(45) a heavy chain variable region having the polypeptide sequence of SEQ ID NO:208, and a light chain variable region having the polypeptide sequence of SEQ ID NO:240;
(46) a heavy chain variable region having the polypeptide sequence of SEQ ID NO:253, and a light chain variable region having the polypeptide sequence of SEQ ID NO:261; or
(47) a heavy chain variable region having the polypeptide sequence of SEQ ID NO:255, and a light chain variable region having the polypeptide sequence of SEQ ID NO:263.

30. The humanized anti-DLL3 monoclonal antibody or antigen-binding fragment thereof of claim 28, wherein the monoclonal antibody or antigen-binding fragment thereof is capable of inducing effector-mediated tumor cell lysis, mediating the recruitment of conjugated drugs, and/or forms a bispecific antibody with another monoclonal antibody or antigen-binding fragment with a cancer-killing effect.

31. An isolated nucleic acid encoding the monoclonal antibody or antigen-binding fragment thereof of claim 28.

32. A vector comprising the isolated nucleic acid of claim 31.

33. A host cell comprising the vector of claim 32.

34. A pharmaceutical composition comprising the isolated monoclonal antibody or antigen-binding fragment thereof of claim 28 and a pharmaceutically acceptable carrier.

35. A method of targeting DLL3 on a cancer cell surface or a method of treating cancer in a subject in need thereof, comprising administering to the subject in need thereof the pharmaceutical composition of claim 34.

36. (canceled)

37. The method of claim 35, wherein the cancer is selected from a lung cancer such as small cell lung cancer (SCLC), large cell neuroendocrine carcinoma (LCNEC), a gastric cancer, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, and other solid tumors, and a non-Hodgkin's lymphoma (NHL), an acute lymphocytic leukemia (ALL), a chronic lymphocytic leukemia (CLL), a chronic myelogenous leukemia (CIVIL), a multiple myeloma (MM), an acute myeloid leukemia (AML), and other liquid tumors.

38. A method of producing the monoclonal antibody or antigen-binding fragment thereof of claim 28, comprising culturing a cell comprising a nucleic acid encoding the monoclonal antibody or antigen-binding fragment thereof under conditions to produce the monoclonal antibody or antigen-binding fragment thereof, and recovering the antibody or antigen-binding fragment thereof from the cell or culture.

39. A method of producing a pharmaceutical composition comprising the monoclonal antibody or antigen-binding fragment thereof of claim 28, comprising combining the monoclonal antibody or antigen-binding fragment thereof with a pharmaceutically acceptable carrier to obtain the pharmaceutical composition.


SEQUENCE LISTING (286 sequences)

SEQ ID NO: 1 - protein, 118 aa, Artificial Sequence
  13P9A Heavy Chain Variable Region
EVQLQQSGPELVKPGASVKMSCKASGYTFTSYVMHWVKQKPGQGPDWIGYINPYNDATKYNEKFKGKATLTSDKSSSTAYMELSSLTSEDSAVYYCARGGYDYDGDYWGQGTTLTVSS

SEQ ID NO: 2 - protein, 107 aa, Artificial Sequence
  13P9A Light Chain Variable Region
DIQMNQSPSSLSASLGDSITITCHASQNINVWLSWYQQKPGNIPKLLIYKASNLHTGVPSRFSGSGSGTGFTLTISSLQPEDIATYYCQQGQSYPFTFGSGTKLEIK

SEQ ID NO: 3 - protein, 122 aa, Artificial Sequence
  5A16A Heavy Chain Variable Region
EVQLQQSGPELVKPGASVKMSCKASGYTFTRYILHWVKLKPGQGLEWIGYINPYNDGTKYNEKFKGKATLTSDKSSSTAYMELSRLTSYDSAVYYCARDSSGYGGAYAMDFWGQGTSVTVSS

SEQ ID NO: 4 - protein, 107 aa, Artificial Sequence
  5A16A Light Chain Variable Region
DIQMTQSPASLSASVGETVTITCRASGNIHNYLAWYQQKQGRSPQLLVYNAKTLPYGVPSRFSGSGSGTQYSLKINSLQPEDFGSYYCQHFWTTPWTFGGGTKLEIK

SEQ ID NO: 5 - protein, 120 aa, Artificial Sequence
  14L22A Heavy Chain Variable Region
EVQLVESGGGLVKPGGSLKLSCAASGFTFSSYAMSWVRQTPEKRLEWVAAINSNGGNTYYPDTVKDRFTISRDNAKNTLYLQMSSLRSEDTALYYCARHRGGFYYAVDYWGQGTSVTVSS

SEQ ID NO: 6 - protein, 112 aa, Artificial Sequence
  14L22A Light Chain Variable Region
NIMMTQSPSSLAVSAGEKVTMSCKSSQSVLYSSNQKNYLAWYQQKPGQSPKLLIYWASTRESGVPDRFTGSGSGTDFTLTISSVQAEDLAVYYCHQYLSSRTFGGGTKLEIK

SEQ ID NO: 7 - protein, 116 aa, Artificial Sequence
  10P18A Heavy Chain Variable Region
EVQLQQSGPELVKPGASVKISCKASGYSFTGYYIDWVKQSPGKSLEWIGYIYPSNGETSYNQKFKGKATLTVDKSSSTVNMQLNSLTSEDSAVYYCARESYAMDYWGQGTSVTVSS

SEQ ID NO: 8 - protein, 111 aa, Artificial Sequence
  10P18A Light Chain Variable Region
DIVLTQSPASLAVSLGQRATISCRASKSVSTSGYSYMHWYQQKPGQPPKLLIYLASNLESGVPARFSGSGSGTDFTLNIHPVEEEDAATYYCQHSRELPYTFGGGTKLEIK

SEQ ID NO: 9 - protein, 119 aa, Artificial Sequence
  13P11A Heavy Chain Variable Region
DVQLQESGPGLVKPSQTVSLTCTVTGYSITNGNHWWSWIRQVSGSKLEWMGYISSSGSTDSNPSLKSRISITRDTSKNQLFLHLNSVTTEDIATYYCATTGTWGYFDYWGQGTTLTVSS

SEQ ID NO: 10 - protein, 107 aa, Artificial Sequence
  13P11A Light Chain Variable Region
NIVMTQSPKSMSMSVGERVTLSCKASENVGTYVSWYQQKPEQSPKLLIYGASNRFTGVPDRFTGSGSATDFTLTISSVQAEDLADYHCGQSYSYPFTFGSGTKLEIK

SEQ ID NO: 11 - protein, 118 aa, Artificial Sequence
  3C16A Heavy Chain Variable Region
EVQLQQSGPELVKPGTSVKMSCKASGYTFTSYVMHWVKQKPGQGLEWIGYVIPYNDGTKYNEKFKGKATLTSDKSSSTAYMELSSLTSEDSAVYYCARPSNWDEFDYWGQGTTLTVSS

SEQ ID NO: 12 - protein, 107 aa, Artificial Sequence
  3C16A Light Chain Variable Region
DIVMTQSQKFMSTSVGDRVSITCKASQNVRTAVAWYQQKPGQSPKALIYLASNRHTGVPDRFTGSGSGTDFTLTISNVQSEDLADYFCLQHWNYPLTFGAGTKLELK

SEQ ID NO: 13 - protein, 116 aa, Artificial Sequence
  3I21A Heavy Chain Variable Region
QVQLQQPGAELVKPGASVKLSCKASGYTFTNYWMNWVKQRPGRGLEWIGRIHPSDSETHYNQKFKTKATLTVDKSSSTAYIQLSSLTSEDSAVYYCARYDGYFAYWGQGTLVTVSA

SEQ ID NO: 14 - protein, 107 aa, Artificial Sequence
  3I21A Light Chain Variable Region
DIQMTQSSSYLSVSLGGRVTITCKASDHINNWLAWYQQKPGNAPRLLISGATSLETGDPSRFSGSGSGKDYTLSITSLQIEDVATYYCQQYWSIPFTFGAGTKLELK

SEQ ID NO: 15 - protein, 120 aa, Artificial Sequence
  8H5A Heavy Chain Variable Region
QVTLKESGPGILQPSQTLSLTCSFSGFSLSTFGMGVGWIRQPSGKGLEWLAHIWWDDDKYYNPALKSRLTISKDTSKNQVFLKIANVDIADTATYYCARTYDYDEYFDYWGQGTTLTVSS

SEQ ID NO: 16 - protein, 112 aa, Artificial Sequence
  8H5A Light Chain Variable Region
DIVMTQAAFSNPVTLGTSASISCRSSKSLLHSNGITYFYWYLQKPGQSPQLLIYQMSNLASGVPDRFSSSGSGTDFTLRISRVEAEDVGVYYCAQNLELPFTFGSGTKLEIK

SEQ ID NO: 17 - protein, 119 aa, Artificial Sequence
  15K2A Heavy Chain Variable Region
QVQLQQPGAELVQPGASVKLSCKASGYTFTSYWMNWMKQRPGRGLEWIGRIHPSDSETHYNQKFRTKATLTVDKSSSTAYIQLSSLTSEDSAVYYCAREDGYYWYFDVWGAGTTVTVSS

SEQ ID NO: 18 - protein, 111 aa, Artificial Sequence
  15K2A Light Chain Variable Region
NIVLTQSPASLAVSLGQRATISCRASESVDIYGNSFMHWYQQKPGQPPKLLIYLASNLESGVPARFSGSGSRTDFTLTIDPVEADDAATYYCQQNNEDPWTFGGGTKLEIK

SEQ ID NO: 19 - protein, 117 aa, Artificial Sequence
  5A24A Heavy Chain Variable Region
EVQLQQSGAELVKPGASVKIPCKASGYKFTDFNMDWVKQSHGKSLEWIGDINPNSGGTIYNQKFKGKATLTVDKSLSTAYMELGSLTSEDTAVYYCARWDYGNFAYWGQGTLVTVSA

SEQ ID NO: 20 - protein, 112 aa, Artificial Sequence
  5A24A Light Chain Variable Region
DIVMTQAAFSNPVTLGTSASISCRSSKSLLHSNGITYLYWYLQKPGQSPQLLIYQMSNLASGVPDRFSSSGSGTDFTLRISRVEAEDVGVYYCAQNLELPLTFGAGTKLELK

SEQ ID NO: 21 - protein, 119 aa, Artificial Sequence
  15P17A Heavy Chain Variable Region
QVQLQQPGAELVKPGASVKLSCKASGYTFTNYWMNWVKQRPGRGLEWIGRIHPSDSETHYNQKFKSKATLTVDKSSSTAYIQLSSLTSEDSAVYYCAREDGYYWYFDVWGAGTTVTVSS

SEQ ID NO: 22 - protein, 111 aa, Artificial Sequence
  15P17A Light Chain Variable Region
NIVLTQSPASLAVSLGQRATISCRASESVDSYGNSFMHWYQQKPGQPPKLLIYLASNLESGVPARFSGSGSRTDFTLTIDPVEADDAATYYCQQNHEDPWTFGGGTKLEIK

SEQ ID NO: 23 - protein, 120 aa, Artificial Sequence
  15N21A Heavy Chain Variable Region
EVQLVESGGGLVKPGGSLKLSCAASGFTFSSYAMSWVRQTPEKRLEWVAAINSNGGRNYYPDTVKDRFTISRDNAKNTLYLQMSSLRSEDTALYYCARHRGGYYYAMDYWGQGTSVTVSS

SEQ ID NO: 24 - protein, 112 aa, Artificial Sequence
  15N21A Light Chain Variable Region
DIVMSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYTYLTFGAGTKLELK

SEQ ID NO: 25 - protein, 8 aa, Artificial Sequence
  13P9A HC CDR1
GYTFTSYV

SEQ ID NO: 26 - protein, 8 aa, Artificial Sequence
  13P9A HC CDR2
INPYNDAT

SEQ ID NO: 27 - protein, 11 aa, Artificial Sequence
  13P9A HC CDR3
ARGGYDYDGDY

SEQ ID NO: 28 - protein, 8 aa, Artificial Sequence
  5A16A HC CDR1
GYTFTRYI

SEQ ID NO: 29 - protein, 8 aa, Artificial Sequence
  5A16A HC CDR2
INPYNDGT

SEQ ID NO: 30 - protein, 15 aa, Artificial Sequence
  5A16A HC CDR3
ARDSSGYGGAYAMDF

SEQ ID NO: 31 - protein, 8 aa, Artificial Sequence
  14L22A HC CDR1
GFTFSSYA

SEQ ID NO: 32 - protein, 8 aa, Artificial Sequence
  14L22A HC CDR2
INSNGGNT

SEQ ID NO: 33 - protein, 13 aa, Artificial Sequence
  14L22A HC CDR3
ARHRGGFYYAVDY

SEQ ID NO: 34 - protein, 8 aa, Artificial Sequence
  10P18A HC CDR1
GYSFTGYY

SEQ ID NO: 35 - protein, 8 aa, Artificial Sequence
  10P18A HC CDR2
IYPSNGET

SEQ ID NO: 36 - protein, 9 aa, Artificial Sequence
  10P18A HC CDR3
ARESYAMDY

SEQ ID NO: 37 - protein, 10 aa, Artificial Sequence
  13P11A HC CDR1
GYSITNGNHW

SEQ ID NO: 38 - protein, 7 aa, Artificial Sequence
  13P11A HC CDR2
ISSSGST

SEQ ID NO: 39 - protein, 11 aa, Artificial Sequence
  13P11A HC CDR3
ATTGTWGYFDY

SEQ ID NO: 40 - protein, 8 aa, Artificial Sequence
  3C16A HC CDR1
GYTFTSYV

SEQ ID NO: 41 - protein, 8 aa, Artificial Sequence
  3C16A HC CDR2
VIPYNDGT

SEQ ID NO: 42 - protein, 11 aa, Artificial Sequence
  3C16A HC CDR3
ARPSNWDEFDY

SEQ ID NO: 43 - protein, 8 aa, Artificial Sequence
  3I21A HC CDR1
GYTFTNYW

SEQ ID NO: 44 - protein, 8 aa, Artificial Sequence
  3I21A HC CDR2
IHPSDSET

SEQ ID NO: 45 - protein, 9 aa, Artificial Sequence
  3I21A HC CDR3
ARYDGYFAY

SEQ ID NO: 46 - protein, 10 aa, Artificial Sequence
  8H5A HC CDR1
GFSLSTFGMG

SEQ ID NO: 47 - protein, 7 aa, Artificial Sequence
  8H5A HC CDR2
IWWDDDK

SEQ ID NO: 48 - protein, 12 aa, Artificial Sequence
  8H5A HC CDR3
ARTYDYDEYFDY

SEQ ID NO: 49 - protein, 8 aa, Artificial Sequence
  15K2A HC CDR1
GYTFTSYW

SEQ ID NO: 50 - protein, 8 aa, Artificial Sequence
  15K2A HC CDR2
IHPSDSET

SEQ ID NO: 51 - protein, 12 aa, Artificial Sequence
  15K2A HC CDR3
AREDGYYWYFDV

SEQ ID NO: 52 - protein, 8 aa, Artificial Sequence
  5A24A HC CDR1
GYKFTDFN

SEQ ID NO: 53 - protein, 8 aa, Artificial Sequence
  5A24A HC CDR2
INPNSGGT

SEQ ID NO: 54 - protein, 10 aa, Artificial Sequence
  5A24A HC CDR3
ARWDYGNFAY

SEQ ID NO: 55 - protein, 8 aa, Artificial Sequence
  15P17A HC CDR1
GYTFTNYW

SEQ ID NO: 56 - protein, 8 aa, Artificial Sequence
  15P17A HC CDR2
IHPSDSET

SEQ ID NO: 57 - protein, 12 aa, Artificial Sequence
  15P17A HC CDR3
AREDGYYWYFDV

SEQ ID NO: 58 - protein, 8 aa, Artificial Sequence
  15N21A HC CDR1
GFTFSSYA

SEQ ID NO: 59 - protein, 8 aa, Artificial Sequence
  15N21A HC CDR2
INSNGGRN

SEQ ID NO: 60 - protein, 13 aa, Artificial Sequence
  15N21A HC CDR3
ARHRGGYYYAMDY

SEQ ID NO: 61 - protein, 6 aa, Artificial Sequence
  13P9A LC CDR1
QNINVW

SEQ ID NO: 62 - protein, 3 aa, Artificial Sequence
  13P9A LC CDR2
KAS

SEQ ID NO: 63 - protein, 9 aa, Artificial Sequence
  13P9A LC CDR3
QQGQSYPFT

SEQ ID NO: 64 - protein, 6 aa, Artificial Sequence
  5A16A LC CDR1
GNIHNY

SEQ ID NO: 65 - protein, 3 aa, Artificial Sequence
  5A16A LC CDR2
NAK

SEQ ID NO: 66 - protein, 9 aa, Artificial Sequence
  5A16A LC CDR3
QHFWTTPWT

SEQ ID NO: 67 - protein, 12 aa, Artificial Sequence
  14L22A LC CDR1
QSVLYSSNQKNY

SEQ ID NO: 68 - protein, 3 aa, Artificial Sequence
  14L22A LC CDR2
WAS

SEQ ID NO: 69 - protein, 8 aa, Artificial Sequence
  14L22A LC CDR3
HQYLSSRT

SEQ ID NO: 70 - protein, 10 aa, Artificial Sequence
  10P18A LC CDR1
KSVSTSGYSY

SEQ ID NO: 71 - protein, 3 aa, Artificial Sequence
  10P18A LC CDR2
LAS

SEQ ID NO: 72 - protein, 9 aa, Artificial Sequence
  10P18A LC CDR3
QHSRELPYT

SEQ ID NO: 73 - protein, 6 aa, Artificial Sequence
  13P11A LC CDR1
ENVGTY

SEQ ID NO: 74 - protein, 3 aa, Artificial Sequence
  13P11A LC CDR2
GAS

SEQ ID NO: 75 - protein, 9 aa, Artificial Sequence
  13P11A LC CDR3
GQSYSYPFT

SEQ ID NO: 76 - protein, 6 aa, Artificial Sequence
  3C16A LC CDR1
QNVRTA

SEQ ID NO: 77 - protein, 3 aa, Artificial Sequence
  3C16A LC CDR2
LAS

SEQ ID NO: 78 - protein, 9 aa, Artificial Sequence
  3C16A LC CDR3
LQHWNYPLT

SEQ ID NO: 79 - protein, 6 aa, Artificial Sequence
  3I21A LC CDR1
DHINNW

SEQ ID NO: 80 - protein, 3 aa, Artificial Sequence
  3I21A LC CDR2
GAT

SEQ ID NO: 81 - protein, 9 aa, Artificial Sequence
  3I21A LC CDR3
QQYWSIPFT

SEQ ID NO: 82 - protein, 11 aa, Artificial Sequence
  8H5A LC CDR1
KSLLHSNGITY

SEQ ID NO: 83 - protein, 3 aa, Artificial Sequence
  8H5A LC CDR2
QMS

SEQ ID NO: 84 - protein, 9 aa, Artificial Sequence
  8H5A LC CDR3
AQNLELPFT

SEQ ID NO: 85 - protein, 10 aa, Artificial Sequence
  15K2A LC CDR1
ESVDIYGNSF

SEQ ID NO: 86 - protein, 3 aa, Artificial Sequence
  15K2A LC CDR2
LAS

SEQ ID NO: 87 - protein, 9 aa, Artificial Sequence
  15K2A LC CDR3
QQNNEDPWT

SEQ ID NO: 88 - protein, 11 aa, Artificial Sequence
  5A24A LC CDR1
KSLLHSNGITY

SEQ ID NO: 89 - protein, 3 aa, Artificial Sequence
  5A24A LC CDR2
QMS

SEQ ID NO: 90 - protein, 9 aa, Artificial Sequence
  5A24A LC CDR3
AQNLELPLT

SEQ ID NO: 91 - protein, 10 aa, Artificial Sequence
  15P17A LC CDR1
ESVDSYGNSF

SEQ ID NO: 92 - protein, 3 aa, Artificial Sequence
  15P17A LC CDR2
LAS

SEQ ID NO: 93 - protein, 9 aa, Artificial Sequence
  15P17A LC CDR3
QQNHEDPWT

SEQ ID NO: 94 - protein, 12 aa, Artificial Sequence
  15N21A LC CDR1
QSLLYSSNQKNY

SEQ ID NO: 95 - protein, 3 aa, Artificial Sequence
  15N21A LC CDR2
WAS

SEQ ID NO: 96 - protein, 8 aa, Artificial Sequence
  15N21A LC CDR3
QQYYTYLT

SEQ ID NO: 97 - protein, 5 aa, Artificial Sequence
  13P9A HC CDR1
SYVMH

SEQ ID NO: 98 - protein, 17 aa, Artificial Sequence
  13P9A HC CDR2
YINPYNDATKYNEKFKG

SEQ ID NO: 99 - protein, 9 aa, Artificial Sequence
  13P9A HC CDR3
GGYDYDGDY

SEQ ID NO: 100 - protein, 5 aa, Artificial Sequence
  5A16A HC CDR1
RYILH

SEQ ID NO: 101 - protein, 17 aa, Artificial Sequence
  5A16A HC CDR2
YINPYNDGTKYNEKFKG

SEQ ID NO: 102 - protein, 13 aa, Artificial Sequence
  5A16A HC CDR3
DSSGYGGAYAMDF

SEQ ID NO: 103 - protein, 5 aa, Artificial Sequence
  14L22A HC CDR1
SYAMS

SEQ ID NO: 104 - protein, 17 aa, Artificial Sequence
  14L22A HC CDR2
AINSNGGNTYYPDTVKD

SEQ ID NO: 105 - protein, 11 aa, Artificial Sequence
  14L22A HC CDR3
HRGGFYYAVDY

SEQ ID NO: 106 - protein, 5 aa, Artificial Sequence
  10P18A HC CDR1
GYYID

SEQ ID NO: 107 - protein, 17 aa, Artificial Sequence
  10P18A HC CDR2
YIYPSNGETSYNQKFKG

SEQ ID NO: 108 - protein, 7 aa, Artificial Sequence
  10P18A HC CDR3
ESYAMDY

SEQ ID NO: 109 - protein, 7 aa, Artificial Sequence
  13P11A HC CDR1
NGNHWWS

SEQ ID NO: 110 - protein, 16 aa, Artificial Sequence
  13P11A HC CDR2
YISSSGSTDSNPSLKS

SEQ ID NO: 111 - protein, 9 aa, Artificial Sequence
  13P11A HC CDR3
TGTWGYFDY

SEQ ID NO: 112 - protein, 5 aa, Artificial Sequence
  3C16A HC CDR1
SYVMH

SEQ ID NO: 113 - protein, 17 aa, Artificial Sequence
  3C16A HC CDR2
YVIPYNDGTKYNEKFKG

SEQ ID NO: 114 - protein, 9 aa, Artificial Sequence
  3C16A HC CDR3
PSNWDEFDY

SEQ ID NO: 115 - protein, 5 aa, Artificial Sequence
  3I21A HC CDR1
NYWMN

SEQ ID NO: 116 - protein, 17 aa, Artificial Sequence
  3I21A HC CDR2
RIHPSDSETHYNQKFKT

SEQ ID NO: 117 - protein, 7 aa, Artificial Sequence
  3I21A HC CDR3
YDGYFAY

SEQ ID NO: 118 - protein, 7 aa, Artificial Sequence
  8H5A HC CDR1
TFGMGVG

SEQ ID NO: 119 - protein, 16 aa, Artificial Sequence
  8H5A HC CDR2
HIWWDDDKYYNPALKS

SEQ ID NO: 120 - protein, 10 aa, Artificial Sequence
  8H5A HC CDR3
TYDYDEYFDY

SEQ ID NO: 121 - protein, 5 aa, Artificial Sequence
  15K2A HC CDR1
SYWMN

SEQ ID NO: 122 - protein, 17 aa, Artificial Sequence
  15K2A HC CDR2
RIHPSDSETHYNQKFRT

SEQ ID NO: 123 - protein, 10 aa, Artificial Sequence
  15K2A HC CDR3
EDGYYWYFDV

SEQ ID NO: 124 - protein, 5 aa, Artificial Sequence
  5A24A HC CDR1
DFNMD

SEQ ID NO: 125 - protein, 17 aa, Artificial Sequence
  5A24A HC CDR2
DINPNSGGTIYNQKFKG

SEQ ID NO: 126 - protein, 8 aa, Artificial Sequence
  5A24A HC CDR3
WDYGNFAY

SEQ ID NO: 127 - protein, 5 aa, Artificial Sequence
  15P17A HC CDR1
NYWMN

SEQ ID NO: 128 - protein, 17 aa, Artificial Sequence
  15P17A HC CDR2
RIHPSDSETHYNQKFKS

SEQ ID NO: 129 - protein, 10 aa, Artificial Sequence
  15P17A HC CDR3
EDGYYWYFDV

SEQ ID NO: 130 - protein, 5 aa, Artificial Sequence
  15N21A HC CDR1
SYAMS

SEQ ID NO: 131 - protein, 17 aa, Artificial Sequence
  15N21A HC CDR2
AINSNGGRNYYPDTVKD

SEQ ID NO: 132 - protein, 11 aa, Artificial Sequence
  15N21A HC CDR3
HRGGYYYAMDY

SEQ ID NO: 133 - protein, 11 aa, Artificial Sequence
  13P9A LC CDR1
HASQNINVWLS

SEQ ID NO: 134 - protein, 7 aa, Artificial Sequence
  13P9A LC CDR2
KASNLHT

SEQ ID NO: 135 - protein, 9 aa, Artificial Sequence
  13P9A LC CDR3
QQGQSYPFT

SEQ ID NO: 136 - protein, 11 aa, Artificial Sequence
  5A16A LC CDR1
RASGNIHNYLA

SEQ ID NO: 137 - protein, 7 aa, Artificial Sequence
  5A16A LC CDR2
NAKTLPY

SEQ ID NO: 138 - protein, 9 aa, Artificial Sequence
  5A16A LC CDR3
QHFWTTPWT

SEQ ID NO: 139 - protein, 17 aa, Artificial Sequence
  14L22A LC CDR1
KSSQSVLYSSNQKNYLA

SEQ ID NO: 140 - protein, 7 aa, Artificial Sequence
  14L22A LC CDR2
WASTRES

SEQ ID NO: 141 - protein, 8 aa, Artificial Sequence
  14L22A LC CDR3
HQYLSSRT

SEQ ID NO: 142 - protein, 15 aa, Artificial Sequence
  10P18A LC CDR1
RASKSVSTSGYSYMH

SEQ ID NO: 143 - protein, 7 aa, Artificial Sequence
  10P18A LC CDR2
LASNLES

SEQ ID NO: 144 - protein, 9 aa, Artificial Sequence
  10P18A LC CDR3
QHSRELPYT

SEQ ID NO: 145 - protein, 11 aa, Artificial Sequence
  13P11A LC CDR1
KASENVGTYVS

SEQ ID NO: 146 - protein, 7 aa, Artificial Sequence
  13P11A LC CDR2
GASNRFT

SEQ ID NO: 147 - protein, 9 aa, Artificial Sequence
  13P11A LC CDR3
GQSYSYPFT

SEQ ID NO: 148 - protein, 11 aa, Artificial Sequence
  3C16A LC CDR1
KASQNVRTAVA

SEQ ID NO: 149 - protein, 7 aa, Artificial Sequence
  3C16A LC CDR2
LASNRHT

SEQ ID NO: 150 - protein, 9 aa, Artificial Sequence
  3C16A LC CDR3
LQHWNYPLT

SEQ ID NO: 151 - protein, 11 aa, Artificial Sequence
  3I21A LC CDR1
KASDHINNWLA

SEQ ID NO: 152 - protein, 7 aa, Artificial Sequence
  3I21A LC CDR2
GATSLET

SEQ ID NO: 153 - protein, 9 aa, Artificial Sequence
  3I21A LC CDR3
QQYWSIPFT

SEQ ID NO: 154 - protein, 16 aa, Artificial Sequence
  8H5A LC CDR1
RSSKSLLHSNGITYFY

SEQ ID NO: 155 - protein, 7 aa, Artificial Sequence
  8H5A LC CDR2
QMSNLAS

SEQ ID NO: 156 - protein, 9 aa, Artificial Sequence
  8H5A LC CDR3
AQNLELPFT

SEQ ID NO: 157 - protein, 15 aa, Artificial Sequence
  15K2A LC CDR1
RASESVDIYGNSFMH

SEQ ID NO: 158 - protein, 7 aa, Artificial Sequence
  15K2A LC CDR2
LASNLES

SEQ ID NO: 159 - protein, 9 aa, Artificial Sequence
  15K2A LC CDR3
QQNNEDPWT

SEQ ID NO: 160 - protein, 16 aa, Artificial Sequence
  5A24A LC CDR1
RSSKSLLHSNGITYLY

SEQ ID NO: 161 - protein, 7 aa, Artificial Sequence
  5A24A LC CDR2
QMSNLAS

SEQ ID NO: 162 - protein, 9 aa, Artificial Sequence
  5A24A LC CDR3
AQNLELPLT

SEQ ID NO: 163 - protein, 15 aa, Artificial Sequence
  15P17A LC CDR1
RASESVDSYGNSFMH

SEQ ID NO: 164 - protein, 7 aa, Artificial Sequence
  15P17A LC CDR2
LASNLES

SEQ ID NO: 165 - protein, 9 aa, Artificial Sequence
  15P17A LC CDR3
QQNHEDPWT

SEQ ID NO: 166 - protein, 17 aa, Artificial Sequence
  15N21A LC CDR1
KSSQSLLYSSNQKNYLA

SEQ ID NO: 167 - protein, 7 aa, Artificial Sequence
  15N21A LC CDR2
WASTRES

SEQ ID NO: 168 - protein, 8 aa, Artificial Sequence
  15N21A LC CDR3
QQYYTYLT

SEQ ID NO: 169 - protein, 618 aa, Homo sapiens
MVSPRMSGLLSQTVILALIFLPQTRPAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLLPPALGLLVAAGVAGAALLLVHVRRRGHSQDAGSRLLAGTPEPSVHALPDALNNLRTQEGSGDGPSSSVDWNRPEDVDPQGIYVISAPSIYAREVATPLFPPLHTGRAGQRQHLLFPYPSSILSVK

SEQ ID NO: 170 - protein, 118 aa, Artificial Sequence
  13P9-H1 Heavy Chain Variable Region
EVRLSQSGGQMKKPGESMRLSCRASGYTFTSYVMHWVRQAPGRRPEWIGYINPYNDATKYARKFQGRATLTSDKYSDTAFLELRSLTSDDTAVYYCARGGYDYDGDYWGRGAPVTVSS

SEQ ID NO: 171 - protein, 107 aa, Artificial Sequence
  13P9-L1 Light Chain Variable Region
EIVMTQSPGTLSLSPGERATLSCHASQNINVWLSWYQQKPGQAPRLLIYKASNLHTGIPDRFSGSGSGTDFTLTISRLEPEDFAVYYCQQGQSYPFTFGQGTKVEIK

SEQ ID NO: 172 - protein, 107 aa, Artificial Sequence
  13P9-L2 Light Chain Variable Region
EIVLTQSPGTLSLSPGERATLSCHASQNINVWLSWYQQKPGQAPRLLIYKASNLHTGIPDRFSGSGSGTDFTLTISRLEPEDFAVYYCQQGQSYPFTFGQGTKVEIK

SEQ ID NO: 173 - protein, 107 aa, Artificial Sequence
  13P9-L3 Light Chain Variable Region
EIVMTQSPATLSLSPGETAIISCHASQNINVWLSWYQQRPGQAPRLLIYKASNLHTGIPDRFSGSGWGTDFNLSISNLESGDFGVYYCQQGQSYPFTFGQGTKVEIK

SEQ ID NO: 174 - protein, 107 aa, Artificial Sequence
  13P9-L4 Light Chain Variable Region
EIVMTQSPATLSLSPGETAIISCHASQNINVWLSWYQQRPGQAPRLLIYKASNLHTGIPDRFSGSGWGTDFNLSISNLESGDFGVYYCQQGQSYPWTFGQGTKVEIK

SEQ ID NO: 175 - protein, 122 aa, Artificial Sequence
  5A16-H1 Heavy Chain Variable Region
QVQLVQSGAEVKKPGSSVKVSCKASGYTFTRYILHWVRLAPGQGLEWIGYINPYNDGTKYNEKFKGKATLTSDKSTNTAYMELSSLRSEDTAVYYCARDSSGYGGAYAMDFWGQGTLVTVSS

SEQ ID NO: 176 - protein, 122 aa, Artificial Sequence
  5A16-H2 Heavy Chain Variable Region
QVQLVQSGAEVKKPGASVKVSCKASGYTFTRYILHWVRLAPGQGLEWIGYINPYNDGTKYNQKFKGKATLTSDKSTNTAYMELSSLRSEDTAVYYCARDSSGYGGAYAMDFWGQGTLVTVSS

SEQ ID NO: 177 - protein, 122 aa, Artificial Sequence
  5A16-H3 Heavy Chain Variable Region
QVQLVQSGAEVKKPGESVKVSCKASGYTFTRYILHWVRLAPGQGLEWIGWINPYNDGTQYNEKFKGRATLTSDKSTSTAYMELSSLRSEDTAVYYCARDSSGYGGAYAMDFWGQGTTVTVSS

SEQ ID NO: 178 - protein, 122 aa, Artificial Sequence
  5A16-H4 Heavy Chain Variable Region
QVQLVQSGAEVKKPGASVKVSCKASGYTFTRYILHWVRLAPGQGLEWIGWINPYNDGTQYNEKFKGRATLTSDTSTSTAYMELSSLRSEDTAVYYCARDSSGYGGAYAMDFWGQGTTVTVSS

SEQ ID NO: 179 - protein, 116 aa, Artificial Sequence
  10P18-H1 Heavy Chain Variable Region
QVQLVQSGAEVKKPGSSVKVSCKASGYSFTGYYIDWVRQAPGQGLEWIGYIYPSNGETSYNQKFKGRATLTVDKSTSTVYMELSSLRSEDTAVYYCARESYAMDYWGQGTLVTVSS

SEQ ID NO: 180 - protein, 116 aa, Artificial Sequence
  10P18-H2 Heavy Chain Variable Region
QVQLVQSGAEVKKPGASVKVSCKASGYSFTGYYIDWVRQAPGQGLEWIGYIYPSNGETSYNQKFKGRATLTVDTSTSTVYMELSSLRSEDTAVYYCARESYAMDYWGQGTTVTVSS

SEQ ID NO: 181 - protein, 116 aa, Artificial Sequence
  10P18-H3 Heavy Chain Variable Region
EVQLVESGGGLVQPGGSLRLSCAASGYSFTGYYMDWVRQAPGKGLEWIGDIYPSNGETIYNQEFKGRATLSVDKSKNTVYLQMNSLRAEDTAVYYCARESYAMDYWGQGTLVTVSS

SEQ ID NO: 182 - protein, 116 aa, Artificial Sequence
  10P18-H4 Heavy Chain Variable Region
EVQLVESGGGLVQPGGSLRLSCAASGYSFTGYYMSWVRQAPGKGLEWIGDIYPSNGETIYNQSFKGRATLSVDNSKNTLYLQMNSLRAEDTAVYYCARESYAMDYWGQGTLVTVSS

SEQ ID NO: 183 - protein, 118 aa, Artificial Sequence
  3C16-H1 Heavy Chain Variable Region
QVQLVQSGAEVKKPGSSVKVSCKASGYTFTSYVLHWVRQAPGQGLEWIGWVIPYNDGTQYNEKFKGRATLTSDKSTSTAYMELSSLRSEDTAVYYCARPSNWDEFDYWGQGTTVTVSS

SEQ ID NO: 184 - protein, 118 aa, Artificial Sequence
  3C16-H2 Heavy Chain Variable Region
QVQLVQSGAEVKKPGASVKVSCKASGYTFTSYVLHWVRQAPGQGLEWIGWVIPYNDGTKYNEKFKGRATLTSDKSTSTAYMELSSLRSEDTAVYYCARPSNWDEFDYWGQGTLVTVSS

SEQ ID NO: 185 - protein, 118 aa, Artificial Sequence
  3C16-H3 Heavy Chain Variable Region
EVQFVQSGAEVKKPGASVRVSCEASGYTFTSYVLQWVRQAPGQRLEWIGWVIPYNDGTSYAPQFQGRATLTSDKYTSTAYMHFKNLRSDDTAIYYCARPSNWDEFDYWGQGTLVTVSS

SEQ ID NO: 186 - protein, 116 aa, Artificial Sequence
  3I21-H1 Heavy Chain Variable Region
EVTLKESGPTLVKPTQTLTLTCTASGYTFTNYWVSWVRQPPGKALEWIGHIHPSDSETRYNPSLKSRATLTVDKSKNQAVLTMTNMDPVDTATYYCARYDGYFAYWGQGTLVTVSS

SEQ ID NO: 187 - protein, 116 aa, Artificial Sequence
  3I21-H2 Heavy Chain Variable Region
QVTLKESGPALVKPTQTLTLTCTASGYTFTNYWVSWVRQPPGKALEWIGHIHPSDSETRYNPSLKSRATLTVDTSKNQAVLTMTNMDPVDTATYYCARYDGYFAYWGQGTLVTVSS

SEQ ID NO: 188 - protein, 116 aa, Artificial Sequence
  3I21-H3 Heavy Chain Variable Region
EVQLLESGGGLVQPGGSLRLSCAASGYTFTNYWMSWVRQAPGKGLEWIGAIHPSDSETYYADSVKGRATLSVDKSKNTAYLQMNSLRAEDTAVYYCARYDGYFAYWGQGTLVTVSS

SEQ ID NO: 189 - protein, 116 aa, Artificial Sequence
  3I21-H4 Heavy Chain Variable Region
EVQLLESGGGLVQPGGSLRLSCAASGYTFTNYWMSWVRQAPGKGLEWIGAIHPSDSETYYADSVKGRATLSVDNSKNTAYLQMNSLRAEDTAVYYCARYDGYFAYWGQGTLVTVSS

SEQ ID NO: 190 - protein, 116 aa, Artificial Sequence
  3I21-H5 Heavy Chain Variable Region
EVQLLESGGGLVQPGGSLRLSCAASGYTFTNYWMSWVRQAPGKGLEWIGAIHPSDSETYYADSVKGRATLSVDNSKNTAYLQMNSLRAEDTAVYYCARYDAYFAYWGQGTLVTVSS

SEQ ID NO: 191 - protein, 119 aa, Artificial Sequence
  15K2-H1 Heavy Chain Variable Region
EVQLVESGGGLVQPGRSLRLSCAASGYTFTSYWMHWMRQAPGKGLEWIGGIHPSDSETGYADSVKGRATLSVDKAKNSAYLQMNSLRAEDMALYYCAREDGYYWYFDVWGQGTMVTVSS

SEQ ID NO: 192 - protein, 119 aa, Artificial Sequence
  15K2-H2 Heavy Chain Variable Region
EVQLVQSGAEVKKPGESLKISCRASGYTFTSYWIGWMRQMPGKGLEWIGIIHPSDSETRYSPSFQGQATLSVDKSINTAYLQWSSLKASDTAMYYCAREDGYYWYFDVWGQGTLVTVSS

SEQ ID NO: 193 - protein, 119 aa, Artificial Sequence
  15K2-H3 Heavy Chain Variable Region
QVQLVQSGAEVKKPGSSVKVSCKASGYTFTSYWISWMRQAPGQGLEWIGSIHPSDSETNYAQKFQGRATLTVDKSTSTAYMELSSLRSEDTAVYYCAREDGYYWYFDVWGQGTLVTVSS

SEQ ID NO: 194 - protein, 117 aa, Artificial Sequence
  5A24-H1 Heavy Chain Variable Region
QVQLVQSGAEVKKPGASVKVSCKASGYKFTDFNMHWVRQAPGQGLEWIGNINPNSGGTNYAEKFKNRATLTVDKSISTAYMELSRLRSDDTAVYYCARWDYGNFAYWGQGTLVTVSS

SEQ ID NO: 195 - protein, 117 aa, Artificial Sequence
  5A24-H2 Heavy Chain Variable Region
QVQLVQSGAEVKKPGASVKVSCKASGYKFTDFNMDWVRQAPGQGLEWIGNINPNSGGTNYAEKFKNRATLTVDTSISTAYMELSRLRSDDTAVYYCARWDYGNFAYWGQGTLVTVSS

SEQ ID NO: 196 - protein, 117 aa, Artificial Sequence
  5A24-H3 Heavy Chain Variable Region
QVQLVQSGAEVKKPGASVKVSCKASGYKFTDFNMHWVRQAPGQGLEWIGEINPNSGGTTYNEKFKGKATLTVDKSTSTAYMELSSLRSEDTAVYYCARWDYGNFAYWGQGTLVTVSS

SEQ ID NO: 197 - protein, 117 aa, Artificial Sequence
  5A24-H4 Heavy Chain Variable Region
QVQLVQSGAEVKKPGSSVKVSCKASGYKFTDFNMHWVRQAPGQGLEWIGYINPNSGGTEYNQKFKDKATLTVDKSTNTAYMELSSLRSEDTAVYYCARWDYGNFAYWGQGTLVTVSS

SEQ ID NO: 198 - protein, 119 aa, Artificial Sequence
  15P17-H1 Heavy Chain Variable Region
EVQLVQSGAEVKKPGSSVKVSCKASGYTFTNYWISWVRQAPGQGLEWIGSIHPSDSETNYAQKFQGRATLTVDKSTSTAYMELSSLRSEDTAVYYCAREDGYYWYFDVWGQGTLVTVSS

SEQ ID NO: 199 - protein, 119 aa, Artificial Sequence
  15P17-H2 Heavy Chain Variable Region
EVQLVQSGAEVKKPGSSVKVSCKASGYTFTNYWISWVRQAPGQGLEWIGSIHPSDSETNYAQKFKGRATLTVDKSTSTAYMELSSLRSEDTAVYYCAREDGYYWYFDVWGQGTLVTVSS

SEQ ID NO: 200 - protein, 119 aa, Artificial Sequence
  15P17-H3 Heavy Chain Variable Region
EVTLKESGPTLVKPTQTLTLTCTASGYTFTNYWMNWVRQPPGKALEWIGRIHPSDSETHYNQKFKSRATLTVDKSKNQAVLTMTNMDPVDTATYYCAREDGYYWYFDVWGQGTLVTVSS

SEQ ID NO: 201 - protein, 119 aa, Artificial Sequence
  15P17-H4 Heavy Chain Variable Region
EVTLKESGPTLVKPTQTLTLTCTASGYTFTNYWMNWVRQPPGKALEWIGRIHPSDSETHYNQKFKSRATLTVDTSKNQAVLTMTNMDPVDTATYYCAREDGYYWYFDVWGQGTLVTVSS

SEQ ID NO: 202 - protein, 120 aa, Artificial Sequence
  15N21-H1 Heavy Chain Variable Region
EVQLLESGGGLVQPGGSLRLSCAASGFTFSSYAMSWVRQAPGKGLEWVAAINSNGGRNYYADSVKGRFTISRDNSKNTLYLQMNSLRAEDTAVYYCARHRGGYYYAMDYWGQGTLVTVSS

SEQ ID NO: 203 - protein, 120 aa, Artificial Sequence
  15N21-H2 Heavy Chain Variable Region
EVQLLESGGGLVQPGGSLRLSCAASGFTFSSYAMSWVRQAPGKGLEWVAAINSNGGRNYYPDSVKGRFTISRDNSKNTLYLQMNSLRAEDTAVYYCARHRGGYYYAMDYWGQGTLVTVSS

SEQ ID NO: 204 - protein, 120 aa, Artificial Sequence
  15N21-H3 Heavy Chain Variable Region
EVQLVESGGGLVQPGGSLRLSCVASGFTFSSYAMSWVRQAPGKGLEWVASINSNGGRNYYSDSVKGRFTISRDNSKNTLYLQMNSLRAEDTAVYYCARHRGGYYYAMDYWGQGTLVTVSS

SEQ ID NO: 205 - protein, 120 aa, Artificial Sequence
  8H5-H1 Heavy Chain Variable Region
EVTLKESGPTLVKPTQTLTLTCTFSGFSLSTFGMGVSWIRQPPGKALEWLAHIWWDDDKRYNPSLKSRLTITKDTSKNQVVLTMTNMDPVDTATYYCARTYDYDEYFDYWGQGTLVTVSS

SEQ ID NO: 206 - protein, 120 aa, Artificial Sequence
  8H5-H2 Heavy Chain Variable Region
QVTLKESGPTLVKPTQTLTLTCTFSGFSLSTFGMGVGWIRQPPGKALEWLAHIWWDDDKRYNPSLKSRLTITKDTSKNQVVLTMTNMDPVDTATYYCARTYDYDEYFDYWGQGTLVTVSS

SEQ ID NO: 207 - protein, 120 aa, Artificial Sequence
  8H5-H3 Heavy Chain Variable Region
EVTLKESGPVLVKPTETLTLTCTFSGFSLSTFGMGVGWIRQPPGKALEWLAHIWWDDDKRYNPALKSRLTISKDTSKSQVVLTMTNMDPVDTATYYCARTYDYDEYFDYWGQGTLVTVSS

SEQ ID NO: 208 - protein, 120 aa, Artificial Sequence
  14L22-H1 Heavy Chain Variable Region
EVQLLESGGGLVQPGGSLRLSCAASGFTFSSYAMSWVRQAPGKGLEWVAAINSNGGNTYYADSVKGRFTISRDNSKNTLYLQMNSLRAEDTAVYYCARHRGGFYYAVDYWGQGTLVTVSS

SEQ ID NO: 209 - protein, 120 aa, Artificial Sequence
  14L22-H2 Heavy Chain Variable Region
QVELVESGGGLVQPGGSLRLSCAASGFTFSSYAMSWVRQAPGKGLEWVAAINSNGGNTYYADSVKGRFTISRDNSKNTLYLQMNSLRAEDTAVYYCARHRGGFYYAVDYWGQGTLVTVSS

SEQ ID NO: 210 - protein, 107 aa, Artificial Sequence
  5A16-L1 Light Chain Variable Region
DIQMTQSPSTLSASVGDRVTITCRASGNIHNYLAWYQQKPGKAPKLLVYNAKTLPYGVPARFSGSGSGTEYTLTISSLQPDDFATYYCQHFWTTPWTFGQGTKVEVK

SEQ ID NO: 211 - protein, 107 aa, Artificial Sequence
  5A16-L2 Light Chain Variable Region
DIQMTQSPSTLSASVGDRVTITCRASGNIHNYLAWYQQKQGKAPKLLVYNAKTLPYGVPARFSGSGSGTEYTLTISSLQPDDFATYYCQHFWTTPWTFGGGTKVEVK

SEQ ID NO: 212 - protein, 107 aa, Artificial Sequence
  5A16-L3 Light Chain Variable Region
DIQMTQSPSSLSASVGDRVTITCKASGNIHNYLAWYQQKPGKAPKLLVYNAKYRYSGVPSRFSGSGSGTDYTLTISSLQPEDFATYYCQHFWTTPWTFGQGTKVEIK

SEQ ID NO: 213 - protein, 111 aa, Artificial Sequence
  10P18-L1 Light Chain Variable Region
DIQLTQSPSSVSASVGDRVTITCRASKSVSTSGYSYMHWYQQKPGKAPKLLIYLASNLESGVPSRFSGSGSGTDFTLTISSLQPEDFATYYCQHSRELPYTFGQGTKVEIK

SEQ ID NO: 214 - protein, 111 aa, Artificial Sequence
  10P18-L2 Light Chain Variable Region
DIVLTQSPDSLAVSLGERATINCRASKSVSTSGYSYLAWYQQKPGQPPKLLIYLASNLESGVPDRFSGSGSGTDFTLTISSLQAEDVAVYYCQHSRELPYTFGGGTKVEIK

SEQ ID NO: 215 - protein, 111 aa, Artificial Sequence
  10P18-L3 Light Chain Variable Region
RIQLTQSPSSLSASVGDRVTITCKASKSVSTSGYSYVHWYQQKPGKAPKLLIYLASYRYTGVPSRFSGSGSGTDFTLTISSLQPEDFATYYCQHSRELPYTFGQGTKVEIK

SEQ ID NO: 216 - protein, 107 aa, Artificial Sequence
  3C16-L1 Light Chain Variable Region
DIQMTQSPSSLSASVGDRVTITCKASQNVRTAVAWYQQKPGKAPKALIYLASYRYSGVPSRFSGSGSGTDFTLTISSLQPEDFATYFCLQHWNYPLTFGQGTKVEIK

SEQ ID NO: 217 - protein, 107 aa, Artificial Sequence
  3C16-L2 Light Chain Variable Region
DIQMTQSPSSLSASVGDRVTITCKASQNVRTALAWYQQKPGKAPKALIYLASNRYSGVPSRFSGSGSGTDFTLTISSLQPEDFATYFCLQHWNYPLTFGQGTKVEIK

SEQ ID NO: 218 - protein, 107 aa, Artificial Sequence
  3C16-L3 Light Chain Variable Region
EIVMTQSPVTVSVSRGGTATLSCRASQNVRTAVAWYQQKPGQTPRALIYLASTRASGVPERFSGSGFGTDFTLSISGLQPEDVAIYFCLQHWNYPLTFGQGTKVEIK

SEQ ID NO: 219 - protein, 107 aa, Artificial Sequence
  3I21-L1 Light Chain Variable Region
DIVMTQSPDSLAVSLGERATINCRASDHINNWMAWYQQKPGQPPKLLISGATNPESGVPDRFSGSGSGKDYTLTISSLQAEDVAVYYCQQYWSIPFTFGQGTKVEIK

SEQ ID NO: 220 - protein, 107 aa, Artificial Sequence
  3I21-L2 Light Chain Variable Region
DIVMTQSPDSLAVSLGERATINCKASDHINNWLAWYQQKPGQPPKLLISGATTRESGVPDRFSGSGSGKDYTLTISSLQAEDVAVYYCQQYWSIPFTFGQGTRLEIK

SEQ ID NO: 221 - protein, 111 aa, Artificial Sequence
  15K2-L1 Light Chain Variable Region
DIQLTQSPSTLSASVGDRVTITCRASESVDIYGNSFLHWYQQKPGKVPKLLIYLASSLESGVPSRFSGSGSRTEFTLTISSLQPDDFATYYCQQNNEDPWTFGPGTKVDIK

SEQ ID NO: 222 - protein, 111 aa, Artificial Sequence
  15K2-L2 Light Chain Variable Region
DIQLTQSPSTLSASVGDRVTITCRASESVDIYGNSFLAWYQQKPGKVPKLLIYLASSLESGVPSRFSGSGSGTEFTLTISSLQPDDFATYYCQQNNEDPWTFGPGTKVDIK

SEQ ID NO: 223 - protein, 111 aa, Artificial Sequence
  15K2-L3 Light Chain Variable Region
DIQLTQSPSSLSASVGDRVTITCRASESVDIYGNSFLHWYQQKPGKTPKLLIYLASSLQSGVPSRFSGSGSRTDFTLTISSLQPEDFATYYCQQNNEDPWTFGQGTKVEIK

SEQ ID NO: 224 - protein, 111 aa, Artificial Sequence
  15K2-L4 Light Chain Variable Region
DIQLTQSPSSLSASVGDRVTITCRASESVDIYGNSFLHWYQQKPGKTPKLLIYLASSLQSGVPSRFSGSGSGTDFTLTISSLQPEDFATYYCQQNNEDPWTFGQGTKVEIK

SEQ ID NO: 225 - protein, 112 aa, Artificial Sequence
  5A24-L1 Light Chain Variable Region
DIVMTQSPLSLPVTPGEPASISCRSSKSLLHSNGITYLYWYLQKPGQSPQLLIYQMSDRFSGVPDRFSSSGSGTDFTLKISRVEAEDVGVYYCAQNLELPLTFGQGTKVEIK

SEQ ID NO: 226 - protein, 112 aa, Artificial Sequence
  5A24-L2 Light Chain Variable Region
DIVMTQTPLSLSVTPGQPASISCRSSKSLLHSNGITYLYWYLQKPGQSPKLLIYQMSYRFSGVPDRFSSSGSGTDFTLKISRVEAEDVGVYYCAQNLELPLTFGQGTKLEIK

SEQ ID NO: 227 - protein, 112 aa, Artificial Sequence
  5A24-L3 Light Chain Variable Region
DIQMTQSPSTLSASVGDRVTITCSSSKSLLHSNGITYMYWYQQKPGKAPKLLIYQMSNLASGVPARFSSSGSGTEFTLTISSLQPDDFATYYCAQNLELPLTFGQGTKVEVK

SEQ ID NO: 228 - protein, 112 aa, Artificial Sequence
  5A24-L4 Light Chain Variable Region
DIQMTQSPSTLSASVGDRVTITCSSSKSLLHSNGITYLAWYQQKPGKAPKLLIYQMSNLASGVPARFSSSGSGTEFTLTISSLQPDDFATYYCAQNLELPLTFGQGTKVEVK

SEQ ID NO: 229 - protein, 111 aa, Artificial Sequence
  15P17-L1 Light Chain Variable Region
DIQLTQSPSSVSASVGDRVTITCRASESVDSYGNSFLHWYQQKPGKAPKLLIYLASSLQSGVPSRFSGSGSRTDFTLTISSLQPEDFATYYCQQNHEDPWTFGQGTKVEIK

SEQ ID NO: 230 - protein, 111 aa, Artificial Sequence
  15P17-L2 Light Chain Variable Region
DIVLTQSPDSLAVSLGERATINCRASESVDSYGNSFMHWYQQKPGQPPKLLIYLASNLESGVPDRFSGSGSRTDFTLTISSLQAEDVAVYYCQQNHEDPWTFGQGTKVEIK

SEQ ID NO: 231 - protein, 111 aa, Artificial Sequence
  15P17-L3 Light Chain Variable Region
DIVLTQSPDSLAVSLGERATINCRASESVDSYGNSFMHWYQQKPGQPPKLLIYLASNLESGVPDRFSGSGSGTDFTLTISSLQAEDVAVYYCQQNHEDPWTFGQGTKVEIK

SEQ ID NO: 232 - protein, 112 aa, Artificial Sequence
  15N21-L1 Light Chain Variable Region
DIVMTQSPDSLAVSLGERATINCKSSQSLLYSSNQKNYLAWYQQKPGQPPKLLIYWASTRESGVPDRFSGSGSGTDFTLTISSLQAEDVAVYYCQQYYTYLTFGQGTKVEIK

SEQ ID NO: 233 - protein, 112 aa, Artificial Sequence
  15N21-L2 Light Chain Variable Region
DIVMTQSPDSLAVSLGERATINCKSSQSLLYSSNQKNYLAWYQQKPGQPPKLLIYWASTRESGVPDRFSGSGSGTDFTLTISSLQAEDVAVYYCQQYYTYLTFGQGTRLEIK

SEQ ID NO: 234 - protein, 112 aa, Artificial Sequence
  15N21-L3 Light Chain Variable Region
DIVMTQSPATLSLSPGERATLSCMSSQSLLYSSNQKNYMAWYQQKPGQAPRLLIYWASTRAPGVPARFSGSGSGTDFTLTISSLEPEDFAVYYCQQYYTYLTFGQGTKVEIK

SEQ ID NO: 235 - protein, 112 aa, Artificial Sequence
  8H5-L1 Light Chain Variable Region
DIVMTQSPDSLAVSLGERATINCRSSKSLLHSNGITYMYWYQQKPGQPPKLLIYQMSNPESGVPDRFSSSGSGTDFTLTISSLQAEDVAVYYCAQNLELPFTFGQGTKVEIK

SEQ ID NO: 236 - protein, 112 aa, Artificial Sequence
  8H5-L2 Light Chain Variable Region
DIVMTQSPDSLAVSLGERATINCKSSKSLLHSNGITYMYWYQQKPGQPPKLLIYQMSNPESGVPDRFSGSGSGTDFTLTISSLQAEDVAVYYCAQNLELPFTFGQGTKVEIK

SEQ ID NO: 237 - protein, 112 aa, Artificial Sequence
  8H5-L3 Light Chain Variable Region
DIVMTQSPDSLAVSLGERATINCKSSKSLLHSNGITYMYWYQQKPGQPPKLLIYQMSTRESGVPDRFSGSGSGTDFTLTISSLQAEDVAVYYCAQNLELPFTFGQGTKVEIK

SEQ ID NO: 238 - protein, 112 aa, Artificial Sequence
  8H5-L4 Light Chain Variable Region
EIVMTQSPATLSLSPGERATLSCRSSKSLLHSNGITYLYWYQQKPGQAPRLLIYQMSTLQSGIPARFSSSGSGTDFTLTISSLEPEDFAVYYCAQNLELPFTFGQGTKLEIK

SEQ ID NO: 239 - protein, 112 aa, Artificial Sequence
  14L22-L1 Light Chain Variable Region
DIVMTQSPDSLAVSLGERATINCKSSQSVLYSSNQKNYLAWYQQKPGQPPKLLIYWASTRESGVPDRFSGSGSGTDFTLTISSLQAEDVAVYYCHQYLSSRTFGQGTRLEIK

SEQ ID NO: 240 - protein, 112 aa, Artificial Sequence
  14L22-L2 Light Chain Variable Region
DIVLTQSPATLSLSPGERATLSCRSSQSVLYSSNQKNYLAWYQQKPGQAPRLLIYWASSRATGVPARFSGSGSGTDFTLTISSLEPEDFATYYCHQYLSSRTFGQGTKVEIK

SEQ ID NO: 241 - protein, 240 aa, Artificial Sequence
  13P9-H1(G4S)3L2
EVRLSQSGGQMKKPGESMRLSCRASGYTFTSYVMHWVRQAPGRRPEWIGYINPYNDATKYARKFQGRATLTSDKYSDTAFLELRSLTSDDTAVYYCARGGYDYDGDYWGRGAPVTVSSGGGGSGGGGSGGGGSEIVLTQSPGTLSLSPGERATLSCHASQNINVWLSWYQQKPGQAPRLLIYKASNLHTGIPDRFSGSGSGTDFTLTISRLEPEDFAVYYCQQGQSYPFTFGQGTKVEIK

SEQ ID NO: 242 - protein, 245 aa, Artificial Sequence
  13P9-H1(G4S)4L2
EVRLSQSGGQMKKPGESMRLSCRASGYTFTSYVMHWVRQAPGRRPEWIGYINPYNDATKYARKFQGRATLTSDKYSDTAFLELRSLTSDDTAVYYCARGGYDYDGDYWGRGAPVTVSSGGGGSGGGGSGGGGSGGGGSEIVLTQSPGTLSLSPGERATLSCHASQNINVWLSWYQQKPGQAPRLLIYKASNLHTGIPDRFSGSGSGTDFTLTISRLEPEDFAVYYCQQGQSYPFTFGQGTKVEIK

SEQ ID NO: 243 - protein, 240 aa, Artificial Sequence
  13P9-L2(G4S)3H1
EIVLTQSPGTLSLSPGERATLSCHASQNINVWLSWYQQKPGQAPRLLIYKASNLHTGIPDRFSGSGSGTDFTLTISRLEPEDFAVYYCQQGQSYPFTFGQGTKVEIKGGGGSGGGGSGGGGSEVRLSQSGGQMKKPGESMRLSCRASGYTFTSYVMHWVRQAPGRRPEWIGYINPYNDATKYARKFQGRATLTSDKYSDTAFLELRSLTSDDTAVYYCARGGYDYDGDYWGRGAPVTVSS

SEQ ID NO: 244 - protein, 244 aa, Artificial Sequence
  5A16-H(G4S)3L
EVQLQQSGPELVKPGASVKMSCKASGYTFTRYILHWVKLKPGQGLEWIGYINPYNDGTKYNEKFKGKATLTSDKSSSTAYMELSRLTSYDSAVYYCARDSSGYGGAYAMDFWGQGTSVTVSSGGGGSGGGGSGGGGSDIQMTQSPASLSASVGETVTITCRASGNIHNYLAWYQQKQGRSPQLLVYNAKTLPYGVPSRFSGSGSGTQYSLKINSLQPEDFGSYYCQHFWTTPWTFGGGTKLEIK

SEQ ID NO: 245 - protein, 245 aa, Artificial Sequence
  15K2-H(G4S)3L
QVQLQQPGAELVQPGASVKLSCKASGYTFTSYWMNWMKQRPGRGLEWIGRIHPSDSETHYNQKFRTKATLTVDKSSSTAYIQLSSLTSEDSAVYYCAREDGYYWYFDVWGAGTTVTVSSGGGGSGGGGSGGGGSNIVLTQSPASLAVSLGQRATISCRASESVDIYGNSFMHWYQQKPGQPPKLLIYLASNLESGVPARFSGSGSRTDFTLTIDPVEADDAATYYCQQNNEDPWTFGGGTKLEIK

SEQ ID NO: 246 - protein, 245 aa, Artificial Sequence
  15P17-H(G4S)3L
QVQLQQPGAELVKPGASVKLSCKASGYTFTNYWMNWVKQRPGRGLEWIGRIHPSDSETHYNQKFKSKATLTVDKSSSTAYIQLSSLTSEDSAVYYCAREDGYYWYFDVWGAGTTVTVSSGGGGSGGGGSGGGGSNIVLTQSPASLAVSLGQRATISCRASESVDSYGNSFMHWYQQKPGQPPKLLIYLASNLESGVPARFSGSGSRTDFTLTIDPVEADDAATYYCQQNHEDPWTFGGGTKLEIK

SEQ ID NO: 247 - protein, 247 aa, Artificial Sequence
  15N21-H(G4S)3L
EVQLVESGGGLVKPGGSLKLSCAASGFTFSSYAMSWVRQTPEKRLEWVAAINSNGGRNYYPDTVKDRFTISRDNAKNTLYLQMSSLRSEDTALYYCARHRGGYYYAMDYWGQGTSVTVSSGGGGSGGGGSGGGGSDIVMSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYTYLTFGAGTKLELK

SEQ ID NO: 248 - protein, 116 aa, Artificial Sequence
  3I21-H6  Heavy Chain Variable Region
EVTLKESGPTLVKPTQTLTLTCKASGYTFTNYWMNWVRQPPGKALEWIGRIHPSDSETHYNPSLKSRATLTVDKSKNQAVLTMTNMDPVDTATYYCARYDGYFAYWGQGTLVTVSS

SEQ ID NO: 249 - protein, 116 aa, Artificial Sequence
  3I21-H7 Heavy Chain Variable Region
EVQLLESGGGLVQPGGSLRLSCKASGYTFTNYWMNWVRQAPGKGLEWIGRIHPSDSETHYNDSVKGRATLSVDKSKNTAYLQMNSLRAEDTAVYYCARYDGYFAYWGQGTLVTVSS

SEQ ID NO: 250 - protein, 119 aa, Artificial Sequence
  15K2-H4 Heavy Chain Variable Region
EVQLVESGGGLVQPGRSLRLSCKASGYTFTSYWMNWMRQAPGKGLEWIGRIHPSDSETHYNDSVKGRATLSVDKAKNSAYLQMNSLRAEDMALYYCAREDGYYWYFDVWGQGTMVTVSS

SEQ ID NO: 251 - protein, 119 aa, Artificial Sequence
  15K2-H5 Heavy Chain Variable Region
EVQLVQSGAEVKKPGESLKISCKASGYTFTSYWMNWMRQMPGKGLEWIGRIHPSDSETHYNPSFQGQATLSVDKSINTAYLQWSSLKASDTAMYYCAREDGYYWYFDVWGQGTLVTVSS

SEQ ID NO: 252 - protein, 119 aa, Artificial Sequence
  15K2-H6 Heavy Chain Variable Region
QVQLVQSGAEVKKPGSSVKVSCKASGYTFTSYWMNWMRQAPGQGLEWIGRIHPSDSETHYNQKFQGRATLTVDKSTSTAYMELSSLRSEDTAVYYCAREDGYYWYFDVWGQGTLVTVSS

SEQ ID NO: 253 - protein, 117 aa, Artificial Sequence
  5A24-H5 Heavy Chain Variable Region
QVQLVQSGAEVKKPGASVKVSCKASGYKFTDFNMDWVRQAPGQGLEWIGDINPNSGGTIYNEKFKNRATLTVDKSISTAYMELSRLRSDDTAVYYCARWDYGNFAYWGQGTLVTVSS

SEQ ID NO: 254 - protein, 120 aa, Artificial Sequence
  8H5-H4 Heavy Chain Variable Region
EVTLKESGPTLVKPTQTLTLTCSFSGFSLSTFGMGVGWIRQPPGKALEWLAHIWWDDDKYYNPSLKSRLTITKDTSKNQVVLTMTNMDPVDTATYYCARTYDYDEYFDYWGQGTLVTVSS

SEQ ID NO: 255 - protein, 120 aa, Artificial Sequence
  8H5-H5 Heavy Chain Variable Region
EVTLKESGPVLVKPTETLTLTCSFSGFSLSTFGMGVGWIRQPPGKALEWLAHIWWDDDKYYNPALKSRLTISKDTSKSQVVLTMTNMDPVDTATYYCARTYDYDEYFDYWGQGTLVTVSS

SEQ ID NO: 256 - protein, 107 aa, Artificial Sequence
  3I21-L3 Light Chain Variable Region
DIVMTQSPDSLAVSLGERATINCRASDHINNWLAWYQQKPGQPPKLLISGATSLESGVPDRFSGSGSGKDYTLTISSLQAEDVAVYYCQQYWSIPFTFGQGTKVEIK

SEQ ID NO: 257 - protein, 107 aa, Artificial Sequence
  3I21-L4 Light Chain Variable Region
DIVMTQSPDSLAVSLGERATINCRASDHINNWLAWYQQKPGQPPKLLISGATSLESGVPDRFSGSGSGKDYTLTISSLQAEDVAVYYCQQYWSIPFTFGQGTRLEIK

SEQ ID NO: 258 - protein, 111 aa, Artificial Sequence
  15K2-L5 Light Chain Variable Region
DIQLTQSPSTLSASVGDRVTITCRASESVDIYGNSFMHWYQQKPGKVPKLLIYLASNLESGVPSRFSGSGSRTEFTLTISSLQPDDFATYYCQQNNEDPWTFGPGTKVDIK

SEQ ID NO: 259 - protein, 111 aa, Artificial Sequence
  15K2-L6 Light Chain Variable Region
DIQLTQSPSSLSASVGDRVTITCRASESVDIYGNSFMHWYQQKPGKTPKLLIYLASNLESGVPSRFSGSGSRTDFTLTISSLQPEDFATYYCQQNNEDPWTFGQGTKVEIK

SEQ ID NO: 260 - protein, 112 aa, Artificial Sequence
  5A24-L5 Light Chain Variable Region
DIVMTQSPLSLPVTPGEPASISCRSSKSLLHSNGITYLYWYLQKPGQSPQLLIYQMSNLASGVPDRFSSSGSGTDFTLKISRVEAEDVGVYYCAQNLELPLTFGQGTKVEIK

SEQ ID NO: 261 - protein, 112 aa, Artificial Sequence
  5A24-L6 Light Chain Variable Region
DIVMTQTPLSLSVTPGQPASISCRSSKSLLHSNGITYLYWYLQKPGQSPKLLIYQMSNLASGVPDRFSSSGSGTDFTLKISRVEAEDVGVYYCAQNLELPLTFGQGTKLEIK

SEQ ID NO: 262 - protein, 112 aa, Artificial Sequence
  5A24-L7 Light Chain Variable Region
DIQMTQSPSTLSASVGDRVTITCRSSKSLLHSNGITYLYWYQQKPGKAPKLLIYQMSNLASGVPARFSSSGSGTEFTLTISSLQPDDFATYYCAQNLELPLTFGQGTKVEVK

SEQ ID NO: 263 - protein, 112 aa, Artificial Sequence
  8H5-L5 Light Chain Variable Region
DIVMTQSPDSLAVSLGERATINCRSSKSLLHSNGITYFYWYQQKPGQPPKLLIYQMSNLASGVPDRFSSSGSGTDFTLTISSLQAEDVAVYYCAQNLELPFTFGQGTKVEIK

SEQ ID NO: 264 - protein, 112 aa, Artificial Sequence
  8H5-L6 Light Chain Variable Region
EIVMTQSPATLSLSPGERATLSCRSSKSLLHSNGITYFYWYQQKPGQAPRLLIYQMSNLASGIPARFSSSGSGTDFTLTISSLEPEDFAVYYCAQNLELPFTFGQGTKLEIK

SEQ ID NO: 265 - protein, 245 aa, Artificial Sequence
  15K2-H5(G4S)3L5
EVQLVQSGAEVKKPGESLKISCKASGYTFTSYWMNWMRQMPGKGLEWIGRIHPSDSETHYNPSFQGQATLSVDKSINTAYLQWSSLKASDTAMYYCAREDGYYWYFDVWGQGTLVTVSSGGGGSGGGGSGGGGSDIQLTQSPSTLSASVGDRVTITCRASESVDIYGNSFMHWYQQKPGKVPKLLIYLASNLESGVPSRFSGSGSRTEFTLTISSLQPDDFATYYCQQNNEDPWTFGPGTKVDIK

SEQ ID NO: 266 - protein, 245 aa, Artificial Sequence
  15K2-H4(G4S)3L5
EVQLVESGGGLVQPGRSLRLSCKASGYTFTSYWMNWMRQAPGKGLEWIGRIHPSDSETHYNDSVKGRATLSVDKAKNSAYLQMNSLRAEDMALYYCAREDGYYWYFDVWGQGTMVTVSSGGGGSGGGGSGGGGSDIQLTQSPSTLSASVGDRVTITCRASESVDIYGNSFMHWYQQKPGKVPKLLIYLASNLESGVPSRFSGSGSRTEFTLTISSLQPDDFATYYCQQNNEDPWTFGPGTKVDIK

SEQ ID NO: 267 - protein, 240 aa, Artificial Sequence
  3C16-H2(G4S)3L3
QVQLVQSGAEVKKPGASVKVSCKASGYTFTSYVLHWVRQAPGQGLEWIGWVIPYNDGTKYNEKFKGRATLTSDKSTSTAYMELSSLRSEDTAVYYCARPSNWDEFDYWGQGTLVTVSSGGGGSGGGGSGGGGSEIVMTQSPVTVSVSRGGTATLSCRASQNVRTAVAWYQQKPGQTPRALIYLASTRASGVPERFSGSGFGTDFTLSISGLQPEDVAIYFCLQHWNYPLTFGQGTKVEIK

SEQ ID NO: 268 - protein, 247 aa, Artificial Sequence
  15N21-H1(G4S)3L1
EVQLLESGGGLVQPGGSLRLSCAASGFTFSSYAMSWVRQAPGKGLEWVAAINSNGGRNYYADSVKGRFTISRDNSKNTLYLQMNSLRAEDTAVYYCARHRGGYYYAMDYWGQGTLVTVSSGGGGSGGGGSGGGGSDIVMTQSPDSLAVSLGERATINCKSSQSLLYSSNQKNYLAWYQQKPGQPPKLLIYWASTRESGVPDRFSGSGSGTDFTLTISSLQAEDVAVYYCQQYYTYLTFGQGTKVEIK

SEQ ID NO: 269 - protein, 252 aa, Artificial Sequence
  15N21-H1(G4S)4L1
EVQLLESGGGLVQPGGSLRLSCAASGFTFSSYAMSWVRQAPGKGLEWVAAINSNGGRNYYADSVKGRFTISRDNSKNTLYLQMNSLRAEDTAVYYCARHRGGYYYAMDYWGQGTLVTVSSGGGGSGGGGSGGGGSGGGGSDIVMTQSPDSLAVSLGERATINCKSSQSLLYSSNQKNYLAWYQQKPGQPPKLLIYWASTRESGVPDRFSGSGSGTDFTLTISSLQAEDVAVYYCQQYYTYLTFGQGTKVEIK

SEQ ID NO: 270 - protein, 247 aa, Artificial Sequence
  15N21-H3(G4S)3L1
EVQLVESGGGLVQPGGSLRLSCVASGFTFSSYAMSWVRQAPGKGLEWVASINSNGGRNYYSDSVKGRFTISRDNSKNTLYLQMNSLRAEDTAVYYCARHRGGYYYAMDYWGQGTLVTVSSGGGGSGGGGSGGGGSDIVMTQSPDSLAVSLGERATINCKSSQSLLYSSNQKNYLAWYQQKPGQPPKLLIYWASTRESGVPDRFSGSGSGTDFTLTISSLQAEDVAVYYCQQYYTYLTFGQGTKVEIK

SEQ ID NO: 271 - protein, 244 aa, Artificial Sequence
  5A16-H1(G4S)3L1
QVQLVQSGAEVKKPGSSVKVSCKASGYTFTRYILHWVRLAPGQGLEWIGYINPYNDGTKYNEKFKGKATLTSDKSTNTAYMELSSLRSEDTAVYYCARDSSGYGGAYAMDFWGQGTLVTVSSGGGGSGGGGSGGGGSDIQMTQSPSTLSASVGDRVTITCRASGNIHNYLAWYQQKPGKAPKLLVYNAKTLPYGVPARFSGSGSGTEYTLTISSLQPDDFATYYCQHFWTTPWTFGQGTKVEVK

SEQ ID NO: 272 - protein, 249 aa, Artificial Sequence
  5A16-H1(G4S)4L1
QVQLVQSGAEVKKPGSSVKVSCKASGYTFTRYILHWVRLAPGQGLEWIGYINPYNDGTKYNEKFKGKATLTSDKSTNTAYMELSSLRSEDTAVYYCARDSSGYGGAYAMDFWGQGTLVTVSSGGGGSGGGGSGGGGSGGGGSDIQMTQSPSTLSASVGDRVTITCRASGNIHNYLAWYQQKPGKAPKLLVYNAKTLPYGVPARFSGSGSGTEYTLTISSLQPDDFATYYCQHFWTTPWTFGQGTKVEVK

SEQ ID NO: 273 - protein, 244 aa, Artificial Sequence
  5A16-H2(G4S)3L3
QVQLVQSGAEVKKPGASVKVSCKASGYTFTRYILHWVRLAPGQGLEWIGYINPYNDGTKYNQKFKGKATLTSDKSTNTAYMELSSLRSEDTAVYYCARDSSGYGGAYAMDFWGQGTLVTVSSGGGGSGGGGSGGGGSDIQMTQSPSSLSASVGDRVTITCKASGNIHNYLAWYQQKPGKAPKLLVYNAKYRYSGVPSRFSGSGSGTDYTLTISSLQPEDFATYYCQHFWTTPWTFGQGTKVEIK

SEQ ID NO: 274 - protein, 244 aa, Artificial Sequence
  5A16-H4(G4S)3L3
QVQLVQSGAEVKKPGASVKVSCKASGYTFTRYILHWVRLAPGQGLEWIGWINPYNDGTQYNEKFKGRATLTSDTSTSTAYMELSSLRSEDTAVYYCARDSSGYGGAYAMDFWGQGTTVTVSSGGGGSGGGGSGGGGSDIQMTQSPSSLSASVGDRVTITCKASGNIHNYLAWYQQKPGKAPKLLVYNAKYRYSGVPSRFSGSGSGTDYTLTISSLQPEDFATYYCQHFWTTPWTFGQGTKVEIK

SEQ ID NO: 275 - protein, 249 aa, Artificial Sequence
  5A16-H4(G4S)4L3
QVQLVQSGAEVKKPGASVKVSCKASGYTFTRYILHWVRLAPGQGLEWIGWINPYNDGTQYNEKFKGRATLTSDTSTSTAYMELSSLRSEDTAVYYCARDSSGYGGAYAMDFWGQGTTVTVSSGGGGSGGGGSGGGGSGGGGSDIQMTQSPSSLSASVGDRVTITCKASGNIHNYLAWYQQKPGKAPKLLVYNAKYRYSGVPSRFSGSGSGTDYTLTISSLQPEDFATYYCQHFWTTPWTFGQGTKVEIK

SEQ ID NO: 276 - protein, 247 aa, Artificial Sequence
  8H5-H5(G4S)3L5
EVTLKESGPVLVKPTETLTLTCSFSGFSLSTFGMGVGWIRQPPGKALEWLAHIWWDDDKYYNPALKSRLTISKDTSKSQVVLTMTNMDPVDTATYYCARTYDYDEYFDYWGQGTLVTVSSGGGGSGGGGSGGGGSDIVMTQSPDSLAVSLGERATINCRSSKSLLHSNGITYFYWYQQKPGQPPKLLIYQMSNLASGVPDRFSSSGSGTDFTLTISSLQAEDVAVYYCAQNLELPFTFGQGTKVEIK

SEQ ID NO: 277 - protein, 247 aa, Artificial Sequence
  8H5-H5(G4S)3L6
EVTLKESGPVLVKPTETLTLTCSFSGFSLSTFGMGVGWIRQPPGKALEWLAHIWWDDDKYYNPALKSRLTISKDTSKSQVVLTMTNMDPVDTATYYCARTYDYDEYFDYWGQGTLVTVSSGGGGSGGGGSGGGGSEIVMTQSPATLSLSPGERATLSCRSSKSLLHSNGITYFYWYQQKPGQAPRLLIYQMSNLASGIPARFSSSGSGTDFTLTISSLEPEDFAVYYCAQNLELPFTFGQGTKLEIK

SEQ ID NO: 278 - protein, 238 aa, Artificial Sequence
  3I21-H7(G4S)3L3
EVQLLESGGGLVQPGGSLRLSCKASGYTFTNYWMNWVRQAPGKGLEWIGRIHPSDSETHYNDSVKGRATLSVDKSKNTAYLQMNSLRAEDTAVYYCARYDGYFAYWGQGTLVTVSSGGGGSGGGGSGGGGSDIVMTQSPDSLAVSLGERATINCRASDHINNWLAWYQQKPGQPPKLLISGATSLESGVPDRFSGSGSGKDYTLTISSLQAEDVAVYYCQQYWSIPFTFGQGTKVEIK

SEQ ID NO: 279 - protein, 242 aa, Artificial Sequence
  10P18-H1(G4S)3L2
QVQLVQSGAEVKKPGSSVKVSCKASGYSFTGYYIDWVRQAPGQGLEWIGYIYPSNGETSYNQKFKGRATLTVDKSTSTVYMELSSLRSEDTAVYYCARESYAMDYWGQGTLVTVSSGGGGSGGGGSGGGGSDIVLTQSPDSLAVSLGERATINCRASKSVSTSGYSYLAWYQQKPGQPPKLLIYLASNLESGVPDRFSGSGSGTDFTLTISSLQAEDVAVYYCQHSRELPYTFGGGTKVEIK

SEQ ID NO: 280 - protein, 242 aa, Artificial Sequence
  10P18-H2(G4S)3L2
QVQLVQSGAEVKKPGASVKVSCKASGYSFTGYYIDWVRQAPGQGLEWIGYIYPSNGETSYNQKFKGRATLTVDTSTSTVYMELSSLRSEDTAVYYCARESYAMDYWGQGTTVTVSSGGGGSGGGGSGGGGSDIVLTQSPDSLAVSLGERATINCRASKSVSTSGYSYLAWYQQKPGQPPKLLIYLASNLESGVPDRFSGSGSGTDFTLTISSLQAEDVAVYYCQHSRELPYTFGGGTKVEIK

SEQ ID NO: 281 - protein, 244 aa, Artificial Sequence
  5A24-H5(G4S)3L5
QVQLVQSGAEVKKPGASVKVSCKASGYKFTDFNMDWVRQAPGQGLEWIGDINPNSGGTIYNEKFKNRATLTVDKSISTAYMELSRLRSDDTAVYYCARWDYGNFAYWGQGTLVTVSSGGGGSGGGGSGGGGSDIVMTQSPLSLPVTPGEPASISCRSSKSLLHSNGITYLYWYLQKPGQSPQLLIYQMSNLASGVPDRFSSSGSGTDFTLKISRVEAEDVGVYYCAQNLELPLTFGQGTKVEIK

SEQ ID NO: 282 - protein, 244 aa, Artificial Sequence
  5A24-H5(G4S)3L7
QVQLVQSGAEVKKPGASVKVSCKASGYKFTDFNMDWVRQAPGQGLEWIGDINPNSGGTIYNEKFKNRATLTVDKSISTAYMELSRLRSDDTAVYYCARWDYGNFAYWGQGTLVTVSSGGGGSGGGGSGGGGSDIQMTQSPSTLSASVGDRVTITCRSSKSLLHSNGITYLYWYQQKPGKAPKLLIYQMSNLASGVPARFSSSGSGTEFTLTISSLQPDDFATYYCAQNLELPLTFGQGTKVEVK

SEQ ID NO: 283 - protein, 245 aa, Artificial Sequence
  15P17-H4(G4S)3L2
EVTLKESGPTLVKPTQTLTLTCTASGYTFTNYWMNWVRQPPGKALEWIGRIHPSDSETHYNQKFKSRATLTVDTSKNQAVLTMTNMDPVDTATYYCAREDGYYWYFDVWGQGTLVTVSSGGGGSGGGGSGGGGSDIVLTQSPDSLAVSLGERATINCRASESVDSYGNSFMHWYQQKPGQPPKLLIYLASNLESGVPDRFSGSGSRTDFTLTISSLQAEDVAVYYCQQNHEDPWTFGQGTKVEIK

SEQ ID NO: 284 - protein, 203 aa, Artificial Sequence
  15P17-H3(G4S)3L1
EVTLKESGPTLVKPTQTLTLTCTASGYTFTNYWMNWVRQPPGKALEWIGRIHPSDSETHYNQKFKSRATLTVDKSKNQAVLTMTNMDPVDTATYYCAREDGYYWYFDVWGQGTLVTVSSGGGGSGGGGSGGGGSDIQLTQSPSSVSASVGDRVTITCRASESVDSYGNSFLHWYQQKPGKAPKLLIYLASSLQSGVPSRFSGS

SEQ ID NO: 285 - protein, 247 aa, Artificial Sequence
  14L22-H1(G4S)3L1
EVQLLESGGGLVQPGGSLRLSCAASGFTFSSYAMSWVRQAPGKGLEWVAAINSNGGNTYYADSVKGRFTISRDNSKNTLYLQMNSLRAEDTAVYYCARHRGGFYYAVDYWGQGTLVTVSSGGGGSGGGGSGGGGSDIVMTQSPDSLAVSLGERATINCKSSQSVLYSSNQKNYLAWYQQKPGQPPKLLIYWASTRESGVPDRFSGSGSGTDFTLTISSLQAEDVAVYYCHQYLSSRTFGQGTRLEIK

SEQ ID NO: 286 - protein, 247 aa, Artificial Sequence
  14L22-H2(G4S)3L1
QVELVESGGGLVQPGGSLRLSCAASGFTFSSYAMSWVRQAPGKGLEWVAAINSNGGNTYYADSVKGRFTISRDNSKNTLYLQMNSLRAEDTAVYYCARHRGGFYYAVDYWGQGTLVTVSSGGGGSGGGGSGGGGSDIVMTQSPDSLAVSLGERATINCKSSQSVLYSSNQKNYLAWYQQKPGQPPKLLIYWASTRESGVPDRFSGSGSGTDFTLTISSLQAEDVAVYYCHQYLSSRTFGQGTRLEIK